(19) United States
(12) Patent Application Publication
(10) Pub. No.: US 2024/0084018 A1
(43) Pub. Date: Mar. 14, 2024

(54) PHARMACEUTICAL FORMULATIONS FOR FUSION PROTEINS

(71) Applicant: BICARA THERAPEUTICS INC., Cambridge, MA (US)

(72) Inventors: Rahul Dnyandeo JANGLE, Bangalore (IN); Kavitha S. RAO, Bangalore (IN); Pradeep JADIYAPPA, Bangalore (IN); Sucharitha JAYAKAR, Bangalore (IN)

(21) Appl. No.: 18/256,748

(22) PCT Filed: Dec. 15, 2021
(86) PCT No.: PCT/US2021/010066

§ 371 Date: Jun. 9, 2023

(30) Foreign Application Priority Data
Dec. 15, 2020 (IN) 202011054539

(51) Int. Cl. C07K 16/28; A61K 47/12; A61K 47/26; A61P 35/00; C07K 14/71

(52) U.S. Cl. CPC C07K 16/2863; A61K 47/12; A61K 47/26; A61P 35/00; C07K 14/71; A61K 38/00

(57) ABSTRACT

Provided herein are liquid pharmaceutical compositions comprising: a fusion protein that comprises a targeting moiety and an immunomodulatory moiety, wherein: i) said targeting moiety specifically binds human epidermal growth factor receptor (hEGFR); and (ii) said immunomodulatory moiety comprises an amino acid sequence of the extracellular domain of human transforming growth factor-beta receptor II (hTGFβRII); a buffer present at a concentration from about 5 mM to about 30 mM; and a tonifying agent present at a concentration from about 4% w/v to about 10% w/v; wherein said liquid pharmaceutical composition has a pH of from about 5.5 to about 7.0. Also provided herein are methods of preparing liquid pharmaceutical composition comprising fusion proteins, and methods of use in treating cancer.

### CROSS-REFERENCE TO RELATED APPLICATIONS

This application claims the priority of Indian Provisional Application No. IN202011054539, filed on Dec. 15, 2020, the entire disclosure of which is incorporated herein by reference.

### BACKGROUND

Therapeutic antibodies are large and complex molecules and, as such, subject to degradation processes, particularly in liquid state. Multifunctional fusion proteins, particularly antibody fusion proteins, are even more complex comprising multiple functional domains of different structure and function either directly connected or connected via a linker. The instabilities in antibodies and fusion proteins make developing a formulation which is stable and suitable for delivery to a subject a challenge. For example, these protein preparations can have short shelf lives and proteins may lose biological activity resulting from e.g., chemical and physical degradation during storage, particularly long-term storage. Chemical degradation processes include for example deamidation, racemization, hydrolysis, oxidation, beta elimination, and disulfide exchange. Physical degradation processes include for example denaturation, aggregation, precipitation, and adsorption. Despite common structural similarities the development of formulations for different monoclonal antibodies has not even been straightforward because of their unique and unpredictable behavior in liquid formulations. This unpredictability is even greater for multifunctional fusion proteins (e.g., fusion proteins comprising an antibody and another protein component) due to e.g., the significant differences in the primary sequence, structure, linkage, multifunctionality, and the relative severity of the degradation pathways (e.g., denaturation, aggregation, surface adsorption, deamidation, oxidation, isomerization, fragmentation, etc.). Accordingly, a need remains for new stable formulations of multifunctional fusion proteins (e.g., those described herein) that exhibit for instance stability, low to undetectable levels of physical or chemical degradation, and little to no loss of the multifunctional biological activity, even after long-term storage.

### SUMMARY

The present invention addresses the above need by providing stable liquid pharmaceutical formulations of multifunctional fusion proteins as described further below (e.g., BCA101). In particular, the present invention provides stable liquid pharmaceutical formulations of the bifunctional fusion proteins disclosed herein. Formulations of the present invention are useful for administration (e.g., via intravenous administration) to mammals, particularly humans suffering from cancer.

Accordingly, in one aspect the instant disclosure provides a liquid pharmaceutical composition comprising: (a) a fusion protein that comprises a targeting moiety and an immunomodulatory moiety, wherein: i) said targeting moiety comprises a polypeptide that specifically binds a membrane bound target protein and has a basic isoelectric point (pI); and (ii) said immunomodulatory moiety comprises a polypeptide that specifically binds a soluble target protein that has an acidic pI; wherein the membrane bound target protein and the soluble target protein are different; (b) a buffer present at a concentration from about 5 mM to about 30 mM; and (c) a tonifying agent present at a concentration from about 4% w/v to about 10% w/v; wherein said liquid pharmaceutical composition has a pH from about 5.5 to about 7.0.

Accordingly, in one aspect the instant disclosure provides a liquid pharmaceutical composition comprising: (a) a fusion protein that comprises a targeting moiety and an immunomodulatory moiety, wherein: i) said targeting moiety specifically binds human epidermal growth factor receptor (hEGFR); and (ii) said immunomodulatory moiety comprises an amino acid sequence of the extracellular domain of human transforming growth factor-beta receptor II (hTGFβRII); (b) a buffer present at a concentration from about 5 mM to about 30 mM; and (c) a tonifying agent present at a concentration from about 4% w/v to about 10% w/v; wherein said liquid pharmaceutical composition has a pH from about 5.5 to about 7.0.

In some embodiments, said buffer is a citrate phosphate buffer, citrate buffer, succinate buffer, or histidine buffer. In some embodiments, said buffer is a citrate phosphate buffer. In some embodiments, said buffer is present at a concentration from about 5 mM to about 25 mM, 5 mM to about 20 mM. 5 mM to about 15 mM, 5 mM to about 10 mM, or 10 mM to about 30 mM. In some embodiments, said buffer is present at a concentration from about 5 mM to about 15 mM. In some embodiments, said buffer is present at a concentration of about 5 mM, 10 mM, 15 mM, 20 mM, 25 mM, or 30 mM. In some embodiments, wherein said buffer is present at a concentration of about 10 mM. In some embodiments, wherein said buffer comprises about 10 mM citrate phosphate.

In some embodiments, said tonifying agent is a saccharide. In some embodiments, said tonifying agent is a disaccharide. In some embodiments, said tonifying agent is sucrose or trehalose. In some embodiments, said tonifying agent is sucrose. In some embodiments, said tonifying agent present at a concentration from about 5% w/v to about 10% w/v, 6% w/v to about 10% w/v, 7% w/v to about 10% w/v, 8% w/v to about 10% w/v, 5% w/v to about 9% w/v, 5% w/v to about 8% w/v, 6% w/v to about 9% w/v, 6% w/v to about 8% w/v, 7% w/v to about 9% w/v, or 7% w/v to about 8% w/v. In some embodiments, said tonifying agent present at a concentration from about 5% w/v to about 8% w/v. In some embodiments, said tonifying agent present at a concentration of about 5% w/v, 6% w/v, 7% w/v, 8% w/v, 9% w/v, or 10% w/v. In some embodiments, said tonifying agent present at a concentration of about 8% w/v. In some embodiments, said tonifying agent is sucrose and is present at a concentration of about 8% w/v.

In some embodiments, said liquid pharmaceutical composition further comprising a surfactant. In some embodiments, said surfactant comprises polysorbate 20, polysorbate 40, polysorbate 60, or polysorbate 80. In some embodiments, said surfactant comprises polysorbate 20. In some embodiments, said surfactant is present at a concentration from about 0.005-0.1% w/v. In some embodiments, said surfactant is present at a concentration from about 0.01-0.1% w/v, 0.02-0.1% w/v, 0.01-0.9% w/v, 0.01-0.8% w/v, 0.01-0.7% w/v, 0.01-0.6% w/v, 0.01-0.5% w/v, 0.01-0.4% w/v, 0.01-0.3% w/v, 0.01-0.2% w/v, 0.01-0.1% w/v, 0.02-0.9% w/v, 0.02-0.8% w/v, 0.02-0.7% w/v, 0.02-0.6% w/v, 0.02-0.5% w/v, 0.02-0.4% w/v, 0.02-0.3% w/v, 0.02-0.2% w/v, 0.02-0.1% w/v, 0.005-0.9% w/v, 0.005-0.8% w/v, 0.005-0.7% w/v, 0.005-0.6% w/v, 0.005-0.5% w/v, 0.005-0.4% w/v, 0.005-0.3% w/v, 0.005-0.2% w/v, or 0.005-0.1% w/v. In some embodiments, said surfactant is present at a concentration of about 0.01% w/v, 0.02% w/v, 0.03% w/v, 0.04% w/v, 0.05% w/v, 0.06% w/v, 0.07% w/v, 0.08% w/v, 0.09% w/v, or 0.1% w/v. In some embodiments, said surfactant is present at a concentration of about 0.02% w/v. In some embodiments, said surfactant is polysorbate 20 and is present at a concentration of about 0.02% w/v.

In some embodiments, said liquid pharmaceutical composition has a pH from about 5.5 to about 7.0, 6.0 to about 7.0, 5.5 to about 6.5, 5.5 to about 6.0, or 6.0 to about 6.5. In some embodiments, said liquid pharmaceutical composition has a pH from about 6.0 to about 6.5. In some embodiments, said liquid pharmaceutical composition has a pH of about 5.5, 6.0, 6.5. or 7.0. In some embodiments, said liquid pharmaceutical composition has a pH of about 6.0.

In some embodiments, said liquid pharmaceutical composition has an osmolality from about 150 mOsmol/kg to about 400 mOsmol/kg. In some embodiments, said liquid pharmaceutical composition has an osmolality from about 150 mOsmol/kg to about 350 mOsmol/kg, 150 mOsmol/kg to about 300 mOsmol/kg, 200 mOsmol/kg to about 400 mOsmol/kg, 250 mOsmol/kg to about 400 mOsmol/kg, 300 mOsmol/kg to about 400 mOsmol/kg, 300 mOsmol/kg to about 350 mOsmol/kg, 250 mOsmol/kg to about 350 mOsmol/kg, or 250 mOsmol/kg to about 300 mOsmol/kg. In some embodiments, said liquid pharmaceutical composition has an osmolality from about 250 mOsmol/kg to about 350 mOsmol/kg. In some embodiments, said liquid pharmaceutical composition has an osmolality of about 250 mOsmol/kg, 300 mOsmol/kg, or 300 mOsmol/kg. In some embodiments, said liquid pharmaceutical composition has an osmolality of about 300 mOsmol/kg.

In some embodiments, said liquid pharmaceutical composition is stable for at least 12, 18, or 24 months when stored at −20° C. In some embodiments, said liquid pharmaceutical composition is stable for at least 12, 18, or 24 months when stored at 2-8° C.

In some embodiments, the concentration of said fusion protein in said liquid pharmaceutical composition is substantially the same for at least 12, 18, or 24 months when stored at −80° C. In some embodiments, the concentration of said fusion protein in said liquid pharmaceutical composition is substantially the same for at least 12, 18, or 24 months when stored at −20° C. In some embodiments, the concentration of said fusion protein in said liquid pharmaceutical composition is substantially the same for at least 12, 18, or 24 months when stored at 2-8° C.

In some embodiments, the concentration of said fusion protein in said liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 12, 18, or 24 months at −80° C. In some embodiments, the concentration of said fusion protein in said liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 12, 18, or 24 months at −20° C. In some embodiments, the concentration of said fusion protein in said liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 12, 18, or 24 months at 2-8° C.

In some embodiments, said liquid pharmaceutical composition is stable upon 1, 2, 3, 4, or 5 cycles of freezing and thawing.

In some embodiments, said fusion protein retains bifunctional activity as measured by bifunctional enzyme-linked immunosorbent assay (ELISA) for at least 12, 18, or 24 months when stored at −20° C. In some embodiments, said fusion protein retains bifunctional activity as measured by bifunctional enzyme-linked immunosorbent assay (ELISA) for at least 12, 18, or 24 months when stored at −20° C. In some embodiments, said fusion protein retains bifunctional activity as measured by bifunctional enzyme-linked immunosorbent assay (ELISA) for at least 12, 18, or 24 months when stored at 2-8° C.

In some embodiments, said liquid pharmaceutical composition comprises less than about 10%, 9%, 8%, 7%, 6%, 5%, 4%, 3%, 2%, or 1% of said fusion protein in aggregate form.

In some embodiments, said liquid pharmaceutical composition has at least one feature selected from the group consisting of (a) increased shelf life, (b) increased temperature stability, (c) decreased formation of aggregates, (d) increased chemical stability, (e) decreased fragmentation, and/or (f) decreased viscosity; after 12, 18, or 24 months of storage at −20° C. or 2-8° C., as compared to a reference formulation.

In some embodiments, said liquid pharmaceutical composition has at least one feature selected from the group consisting of: (a) decreased percentage of aggregates as measured by size exclusion chromatography (SEC), (b) higher percentage of monomers as measured by SEC, and/or (c) lower turbidity value in nephelometry units (NTU); after 12, 18, or 24 months of storage at −20° C. or 2-8° C., as compared to the reference formulation.

In some embodiments, said fusion protein is present at a concentration from about 5-50 mg/ml, 5-40 mg/ml, 5-30 mg/ml, 5-25 mg/ml, 10-50 mg/ml, 20-50 mg/ml, 25-50 mg/ml, 20-50 mg/ml, 20-40 mg/ml, 20-30 mg/ml, 25-50 mg/ml, 25-40 mg/ml, or 25-30 mg/ml. In some embodiments, said fusion protein is present at a concentration from about 20-30 mg/ml. In some embodiments, said fusion protein is present at a concentration of about 5 mg/ml, 10 mg/ml, 15 mg/ml, 20 mg/ml, 25 mg/ml, 30 mg/ml, 35 mg/ml, 40 mg/ml, 45 mg/ml, or 50 mg/ml. In some embodiments, said fusion protein is present at a concentration of about 25 mg/ml.

In some embodiments, said targeting moiety that specifically binds hEGFR comprises an antibody or functional fragment or functional variant thereof, that specifically binds hEGFR. In some embodiments, said antibody, or functional fragment or functional variant thereof, that specifically binds hEGFR is a full-length antibody, a single chain variable fragment (scFv), a scFv2, a scFv-Fc, a Fab, a Fab′, a F(ab′)2, or a F(v).

In some embodiments, said antibody, or functional fragment or functional variant thereof, that specifically binds hEGFR comprises a VH that comprises VH CDR1, VH CDR2, and VH CDR3, wherein (a) VH CDR1 comprises an amino acid sequence at least 95%, 96%, 97%, 98%, 99%, or 100% identical to the amino acid sequence of SEQ ID NO: 1; (b) VH CDR2 comprises an amino acid sequence at least 95%, 96%, 97%, 98%, 99%, or 100% identical to the amino acid sequence of SEQ ID NO: 2; and (c) VH CDR3 comprises an amino acid sequence at least 95%, 96%, 97%, 98%, 99%, or 100% identical to the amino acid sequence of SEQ ID NO: 3.

In some embodiments, said antibody, or functional fragment or functional variant thereof, that specifically binds hEGFR comprises a VL that comprises a VL CDR1, a VL CDR2, and a VL CDR3, wherein (a) VL CDR1 comprises an amino acid sequence at least 95%, 96%, 97%, 98%, 99%, or 100% identical to the amino acid sequence of SEQ ID NO: 4; (b) VL CDR2 comprises an amino acid sequence at least 95%, 96%, 97%, 98%, 99%, or 100% identical to the amino acid sequence of SEQ ID NO: 5; and (c) VL CDR3 comprises an amino acid sequence at least 95%, 96%, 97%, 98%, 99%, or 100% identical to the amino acid sequence of SEQ ID NO: 6.

In some embodiments, said antibody, or functional fragment or functional variant thereof, that specifically binds hEGFR comprises a VH that comprises an amino acid sequence at least 95%, 96%, 97%, 98%, 99%, or 100% identical to the amino acid sequence of SEQ ID NO: 7.

In some embodiments, said antibody, or functional fragment or functional variant thereof, that specifically binds hEGFR comprises a VL that comprises an amino acid sequence at least 95%, 96%, 97%, 98%, 99%, or 100% identical to the amino acid sequence of SEQ ID NO: 8.

In some embodiments, said antibody, or functional fragment or functional variant thereof, that specifically binds hEGFR comprises a heavy chain that comprises an amino acid sequence at least 95%, 96%, 97%, 98%, 99%, or 100% identical to the amino acid sequence of SEQ ID NO: 9.

In some embodiments, said antibody, or functional fragment or functional variant thereof, that specifically binds hEGFR consists of a heavy chain that comprises an amino acid sequence at least 95%, 96%, 97%, 98%, 99%, or 100% identical to the amino acid sequence of SEQ ID NO: 10.

In some embodiments, said antibody, or functional fragment or functional variant thereof, that specifically binds hEGFR comprises a heavy chain that consists of an amino acid sequence at least 95%, 96%, 97%, 98%, 99%, or 100% identical to the amino acid sequence of SEQ ID NO: 9.

In some embodiments, said antibody, or functional fragment or functional variant thereof, that specifically binds hEGFR consists of a heavy chain that consists of an amino acid sequence at least 95%, 96%, 97%, 98%, 99%, or 100% identical to the amino acid sequence of SEQ ID NO: 10.

In some embodiments, said antibody, or functional fragment or functional variant thereof, that specifically binds hEGFR comprises a light chain that comprises an amino acid sequence at least 95%, 96%, 97%, 98%, 99%, or 100% identical to the amino acid sequence of SEQ ID NO: 11.

In some embodiments, said antibody, or functional fragment or functional variant thereof, that specifically binds hEGFR consists of a light chain that consists of an amino acid sequence at least 95%, 96%, 97%, 98%, 99%, or 100% identical to the amino acid sequence of SEQ ID NO: 11.

In some embodiments, said antibody, or functional fragment or functional variant thereof, that specifically binds hEGFR comprises cetuximab or panitumumab, or a functional fragment or functional variant of any of the foregoing.

In some embodiments, said immunomodulatory moiety comprises an amino acid sequence at least 95%, 96%, 97%, 98%, 99%, or 100% identical to the amino acid sequence of SEQ ID NO: 23. In some embodiments, said immunomodulatory moiety consists of an amino acid sequence at least 95%, 96%, 97%, 98%, 99%, or 100% identical to the amino acid sequence of SEQ ID NO: 23.

In some embodiments, said immunomodulatory moiety is indirectly fused to said targeting moiety. In some embodiments, said immunomodulatory moiety is indirectly fused to said targeting moiety via a peptide linker.

In some embodiments, said immunomodulatory moiety is indirectly fused to said targeting moiety via a peptide linker of sufficient length such that said immunomodulatory moiety and said targeting moiety can simultaneously bind the respective targets. In some embodiments, said linker comprises the amino acid sequence of SEQ ID NO: 24, 25, 26, 27, or 28. In some embodiments, said linker comprises the amino acid sequence of SEQ ID NO: 24. In some embodiments, said linker consists of the amino acid sequence of SEQ ID NO: 24.

In some embodiments, said immunomodulatory moiety is fused to the C terminus of said targeting moiety. In some embodiments, said immunomodulatory moiety is fused to the N terminus of said targeting moiety.

In some embodiments, said targeting moiety is an antibody that comprises a light chain and a heavy chain, and wherein said immunomodulatory moiety is fused to the C terminus of said heavy chain of said targeting moiety.

In some embodiments, said targeting moiety is an antibody that comprises a light chain and a heavy chain, and wherein said immunomodulatory moiety is fused to the C terminus of said light chain of said targeting moiety.

In some embodiments, said targeting moiety is an antibody specifically binds hEGFR that comprises a heavy chain that comprises an amino acid sequence at least 95%, 96%, 97%, 98%, 99%, or 100% identical to the amino acid sequence of SEQ ID NO: 10, and a light chain that comprises an amino acid sequence at least 95%, 96%, 97%, 98%, 99%, or 100% identical to the amino acid sequence of SEQ ID NO: 11, and wherein said immunomodulatory moiety comprises an amino acid sequence at least 95%, 96%, 97%, 98%, 99%, or 100% identical to the amino acid sequence of SEQ ID NO: 23, and wherein the N terminus of said immunomodulatory moiety is fused indirectly through a linker to the C terminus of said heavy chain or said light chain, and wherein said linker comprises an amino acid sequence at least 95%, 96%, 97%, 98%, 99%, or 100% identical to the amino acid sequence of SEQ ID NO: 24.

In some embodiments, said targeting moiety is an antibody specifically binds hEGFR that comprises a heavy chain that comprises an amino acid sequence at least 95%, 96%, 97%, 98%, 99%, or 100% identical to the amino acid sequence of SEQ ID NO: 10, and a light chain that comprises an amino acid sequence at least 95%, 96%, 97%, 98%, 99%, or 100% identical to the amino acid sequence of SEQ ID NO: 11, and wherein said immunomodulatory moiety comprises an amino acid sequence at least 95%, 96%, 97%, 98%, 99%, or 100% identical to the amino acid sequence of SEQ ID NO: 23, and wherein the N terminus of said immunomodulatory moiety is fused indirectly through a linker to the C terminus of said light chain, and wherein said linker comprises an amino acid sequence at least 95%, 96%, 97%, 98%, 99%, or 100% identical to the amino acid sequence of SEQ ID NO: 24.

In some embodiments, said targeting moiety comprises an antibody that comprises a heavy chain comprising an amino acid sequence at least 95%, 96%, 97%, 98%, 99%, or 100% identical to the amino acid sequence of SEQ ID NO: 10; and a light chain comprising an amino acid sequence at least 95%, 96%, 97%, 98%, 99%, or 100% identical to the amino acid sequence of SEQ ID NO: 29.

In some embodiments, said liquid pharmaceutical composition is sterile.

In one aspect, provided herein is a liquid pharmaceutical composition comprising: (a) a fusion protein that comprises a targeting moiety and an immunomodulatory moiety, wherein: i) said targeting moiety specifically binds hEGFR; and (ii) said immunomodulatory moiety comprises an amino acid sequence of the extracellular domain of hTGFβRII; (b) from about 5 mM to about 20 mM citrate phosphate buffer; and (c) from about 6% w/v to about 10% w/v sucrose; wherein said liquid pharmaceutical composition has a pH of from about 5.5 to about 6.5.

In some embodiments, said targeting moiety comprises an antibody that comprises a heavy chain comprising an amino acid sequence at least 95%, 96%, 97%, 98%, 99%, or 100% identical to the amino acid sequence of SEQ ID NO: 10; and a light chain comprising an amino acid sequence at least 95%, 96%, 97%, 98%, 99%, or 100% identical to the amino acid sequence of SEQ ID NO: 29.

In some embodiments, said fusion protein is present at a concentration of about 25 mg/ml.

In some embodiments, said liquid pharmaceutical composition further comprising from about 0.01-0.05% w/v polysorbate 20.

In one aspect, provided herein is a liquid pharmaceutical composition comprising: (a) a fusion protein that comprises a targeting moiety and an immunomodulatory moiety, wherein: i) said targeting moiety specifically binds hEGFR; and (ii) said immunomodulatory moiety comprises an amino acid sequence of the extracellular domain of hTGFβRII; (b) about 10 mM citrate phosphate buffer; and (c) about 8% w/v sucrose; wherein said liquid pharmaceutical composition has a pH of about 6.0.

In some embodiments, said pharmaceutical composition further comprising from about 0.02% w/v polysorbate 20.

In some embodiments, said targeting moiety comprises an antibody that comprises a heavy chain comprising an amino acid sequence at least 95%, 96%, 97%, 98%, 99%, or 100% identical to the amino acid sequence of SEQ ID NO: 10; and a light chain comprising an amino acid sequence at least 95%, 96%, 97%, 98%, 99%, or 100% identical to the amino acid sequence of SEQ ID NO: 29.

In some embodiments, said fusion protein is present at a concentration of about 25 mg/ml.

In one aspect, provided herein is a liquid pharmaceutical composition comprising: (a) about 25 mg/mL of a fusion protein that comprises a targeting moiety and an immunomodulatory moiety, wherein said targeting moiety comprises an antibody that comprises a heavy chain comprising an amino acid sequence at least 95%, 96%, 97%, 98%, 99%, or 100% identical to the amino acid sequence of SEQ ID NO: 10; and a light chain comprising an amino acid sequence at least 95%, 96%, 97%, 98%, 99%, or 100% identical to the amino acid sequence of SEQ ID NO: 29; (b) about 10 mM citrate phosphate buffer; (c) about 8% w/v sucrose; and (d) about 0.02% w/v polysorbate 20; wherein said liquid pharmaceutical composition has a pH of about 6.0.

In one aspect, provided herein is a method of treating human cancer in a subject having cancer, said method comprising administering to said subject the liquid pharmaceutical composition described herein.

In some embodiments, said liquid pharmaceutical composition is administered in an amount effective to treat said cancer.

In some embodiments, said fusion protein is administered to said human subject at a dose from about 10 mg to 2000 mg. In some embodiments, said fusion protein is administered to said human subject at a dose from about 20 mg to 1000 mg. In some embodiments, said fusion protein is administered to said human subject at a dose from about 30 mg to 1000 mg. In some embodiments, said fusion protein is administered to said human subject at a dose from about 40 mg to 1000 mg. In some embodiments, said fusion protein is administered to said human subject at a dose from about 50 mg to 1000 mg. In some embodiments, said fusion protein is administered to said human subject at a dose from about 10 mg to 100 mg. In some embodiments, said fusion protein is administered to said human subject at a dose from about 10 mg to 900 mg. In some embodiments, said fusion protein is administered to said human subject at a dose from about 10 mg to 800 mg. In some embodiments, said fusion protein is administered to said human subject at a dose from about 10 mg to 700 mg. In some embodiments, said fusion protein is administered to said human subject at a dose from about 10 mg to 800 mg. In some embodiments, said fusion protein is administered to said human subject at a dose from about 10 mg to 700 mg. In some embodiments, said fusion protein is administered to said human subject at a dose from about 10 mg to 600 mg. In some embodiments, said fusion protein is administered to said human subject at a dose from about 10 mg to 500 mg. In some embodiments, said fusion protein is administered to said human subject at a dose from about 10 mg to 400 mg. In some embodiments, said fusion protein is administered to said human subject at a dose from about 10 mg to 300 mg. In some embodiments, said fusion protein is administered to said human subject at a dose from about 10 mg to 100 mg. In some embodiments, said fusion protein is administered to said human subject at a dose from about 10 mg to 50 mg.

In some embodiments, said fusion protein is administered to said human subject at a dose of about 50 mg, 60 mg, 64 mg, 100 mg, 150 mg, 200 mg, 240 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900, or 2000 mg. In some embodiments, said fusion protein is administered to said human subject at a dose of about 64 mg, 240 mg, 800 mg, or 1600 mg.

In some embodiments, said fusion protein is administered to said human subject every 1, 2, 3, or 4 weeks. In some embodiments, said fusion protein is administered to said human subject every week.

In some embodiments, said fusion protein is administered to said human subject 3 weeks.

In some embodiments, the administering step comprises intravenously injecting the liquid pharmaceutical composition.

In some embodiments, said cancer is a solid tumor. In some embodiments, said cancer is metastatic. In some embodiments, said cancer is recurrent. In some embodiments, said cancer is refractory. In some embodiments, said cancer is metastatic, recurrent, and/or refractory, or any combination thereof.

In some embodiments, said cancer comprises cancer cells that contain a genomic amplification of the EGFR gene, e.g., as detected by biopsy and fluorescence in situ hybridization.

In some embodiments, said cancer comprises cancer cells that contain a genomic modification in the KRAS gene. In some embodiments, said modification in the KRAS gene is a G12D substitution. In some embodiments, said modification in the KRAS gene is a G13D modification.

In some embodiments, said cancer is selected from the group consisting of eye, stomach, colon, rectum, colorectal, breast cancer, anal cancer, pancreatic cancer, thyroid cancer, liver cancer, ovarian cancer, lung cancer, skin cancer, brain cancer, spinal cord cancer, head cancer, and neck cancer.

In some embodiments, said cancer is lung cancer. In some embodiments, said cancer is squamous cell lung cancer (SqCLC). In some embodiments, said SqCLC comprises cancer cells that does not express detectable levels of programmed death-ligand 1, as measured by a biopsy. In some embodiments, said SqCLC comprises cancer cells that contain a genomic amplification of the EGFR gene, e.g., as detected by biopsy and fluorescence in situ hybridization.

In some embodiments, said cancer is colorectal cancer. In some embodiments, said colorectal cancer is RAS wild-type microsatellite stable Colorectal Carcinoma (RAS WT MSS CRC). In some embodiments, said cancer is breast cancer. In some embodiments, said cancer is triple negative breast cancer (TNBC). In some embodiments, said cancer is a spinal cord cancer. In some embodiments, said cancer of the spinal cord is a chordoma. In some embodiments, said cancer is a cancer of the eye. In some embodiments, said cancer of the eye is a melanoma of the eye. In some embodiments, said cancer is a brain cancer. In some embodiments, said brain cancer is a glioblastoma. In some embodiments, said cancer is ovarian cancer. In some embodiments, said ovarian cancer is epithelial ovarian cancer. In some embodiments, said cancer is liver cancer. In some embodiments, said liver cancer is hepatocellular carcinoma (HCC). In some embodiments, said cancer is thyroid cancer. In some embodiments, said thyroid cancer is anaplastic thyroid cancer (ATC). In some embodiments, said cancer is pancreatic cancer. In some embodiments, said cancer is stomach cancer. In some embodiments, said cancer is head and neck cancer. In some embodiments, said cancer is head and neck squamous cell carcinoma (HNSCC). In some embodiments, said cancer is recurrent HNSCC. In some embodiments, said cancer is metastatic HNSCC. In some embodiments, said cancer is recurrent and metastatic HNSCC. In some embodiments, said cancer is squamous cell carcinoma of anal canal (SCCAC). In some embodiments, said cancer is recurrent SCCAC. In some embodiments, said cancer is metastatic SCCAC. In some embodiments, said cancer is recurrent and metastatic SCCAC.

In one aspect, provided herein is a method of making a liquid pharmaceutical composition comprising: (a) culturing mammalian cells having stably incorporated into their genome one or more nucleic acids encoding a fusion protein that comprises a targeting moiety and an immunomodulatory moiety, wherein: i) said targeting moiety specifically binds hEGFR; and (ii) said immunomodulatory moiety comprises an amino acid sequence of the extracellular domain of hTGFβRII in a cell culture medium such that the cells secrete said fusion protein into the cell culture medium; (b) purifying the fusion protein from the cell culture media; and (c) preparing the pharmaceutical composition described herein.

### BRIEF DESCRIPTION OF THE FIGURES

FIG. 1 is a schematic showing the development strategy of formulations described herein.

FIG. 2 is a schematic showing an outline of the pH screening study described in Example 1 and test BCA101 formulations at pH 5.0, 5.5, 6.0, and 6.5.

FIG. 3 is a dot graph showing the percentage of high molecular weight protein (HMWP) in the bulk tangential flow filtration composition (TFF) and the final drug product (FDP) at each test pH (5.0, 5.5, 6.0, and 6.5).

FIG. 4 is a dot graph showing the percentage of low monomeric proteins in the bulk TFF composition and the FDP at each test pH (5.0, 5.5, 6.0, and 6.5).

FIG. 5 is a dot graph showing the percentage of low molecular weight protein (LMWP) in the bulk TFF composition and the FDP at each test pH (5.0, 5.5, 6.0, and 6.5).

FIG. 6 is a series of line graphs showing the results of the differential scanning calorimetry (DSC) analysis described in Example 1 for each BCA101 test formulation evaluated.

FIG. 7 is a dot graph summarizing the results of the DSC analysis described in Example 1 for each test formulation shown in FIG. 6.

FIG. 8A is a line graph showing the percent HMWP at 40° C. for the 6.0 and 6.5 pH test formulations. FIG. 8B is a dot graph showing the percent HMWP slope/week at 40° C. for the 6.0 and 6.5 pH test formulations.

FIG. 9A is a line graph showing the percent monomer at 40° C. for the 6.0 and 6.5 pH test formulations. FIG. 9B is a dot graph showing the percent monomer slope/week at 40° C. for the 0.0 and 6.5 pH test formulations.

FIG. 10A is a line graph showing the percent LMWP at 40° C. for the 6.0 and 6.5 pH test formulations. FIG. 10B is a dot graph showing the percent LMWP slope/week at 40° C. for the 6.0 and 6.5 pH test formulations.

FIG. 11 is a schematic showing the process of the buffer screening study described in Example 1 and the composition of the test formulations evaluated.

FIG. 12A is a line graph showing the results of the DSC analysis for the citrate buffer formulation with a pH of 6.0. FIG. 12B is a line graph showing the results of the DSC analysis for the citrate buffer formulation with a pH of 6.5.

FIG. 13A is a line graph showing the results of the DSC analysis for the succinate buffer formulation with a pH of 6.0. FIG. 13B is a line graph showing the results of the DSC analysis for the succinate buffer formulation with a pH of 6.5.

FIG. 14A is a line graph showing the results of the DSC analysis for the histidine buffer formulation with a pH of 6.0. FIG. 14B is a line graph showing the results of the DSC analysis for the histidine buffer formulation with a pH of 6.5.

FIG. 15A is a line graph showing the results of the DSC analysis for the citrate phosphate buffer formulation with a pH of 6.0. FIG. 15B is a line graph showing the results of the DSC analysis for the citrate phosphate buffer formulation with a pH of 6.5.

FIG. 16 is a dot graph showing a summary of the DSC analysis data presented in FIGS. 12A-15B.

FIG. 17 is a schematic showing the process of the tonicity modifier screening study described in Example 1 examine test formulations comprising sucrose or trehalose.

FIG. 18 is a copy of a photograph of the BCA101 fusion protein in a formulation comprising 25 mg/ml BCA101, 8.0% w/v sucrose, 0.02% w/v polysorbate 20, and 10 mM citrate phosphate buffer (pH 6.0), comparing the color of the BCA101 liquid formulation to the pharmacopoeial (Ph.Eu.2.2.2) color standard solutions.

FIG. 19 is a table showing absorbance at 506 nm of the BCA101 fusion protein in a formulation comprising 25 mg/ml BCA101, 8.0% w/v sucrose, 0.02% w/v polysorbate 20, and 10 mM citrate phosphate buffer (pH 6.0) of a toxicology study batch, an internal reference standard batch (IRS batch), and a dose range finding batch.

FIG. 20 is a copy of a photograph of the BCA101 fusion protein in a formulation comprising 25 mg/ml BCA101, 8.0% w/v sucrose, 0.02% w/v polysorbate 20, and 10 mM citrate phosphate buffer (pH 6.0), comparing the clarity and degree of opalescence of the BCA101 liquid formulation to the pharmacopoeial standard (formazin suspensions, Ph.Eu.2.2.1).

FIG. 21 is a table showing the turbidity in nephelometric turbidity units (NTUs) of the BCA101 fusion protein in a formulation comprising 25 mg/ml BCA101, 8.0% w/v sucrose, 0.02% w/v polysorbate 20, and 10 mM citrate phosphate buffer (pH 6.0). Batches analyzed prior to filtration are indicated with “BF;” and batches analyzed after filtration are indicated as “AF.” The assay was conducted as per Ph. Eur. 2.2.1, the full contents of which are incorporated by reference herein.

FIG. 22 is a series of dot graphs showing the pH, osmolarity, protein concentration, and bifunctional capability of BCA101 drug substance (DS) formulated in a liquid formulation comprising 25 mg/ml BCA101, 8.0% w/v sucrose, 0.02% w/v polysorbate 20, and 10 mM citrate phosphate buffer (pH 6.0) over the course of 24 months stored in 5 mL Celsius bags at −20° C. Samples were analyzed at the initial time point, 1 month, 2 months, 3 months, 6 months, 12 months, 18 months, and 24 months.

FIG. 23 is a series of dot graphs showing the pH, osmolarity, protein concentration, and bifunctional capability of BCA101 drug product (DP) formulated in a liquid formulation comprising 25 mg/ml BCA101, 8.0% w/v sucrose, 0.02% w/v polysorbate 20, and 10 mM citrate phosphate buffer (pH 6.0) over the course of 24 months stored in 5 mL Celsius bags at −20° C. Samples were analyzed at the initial time point, 1 month, 2 months, 3 months, 6 months, 12 months, 18 months, and 24 months.

FIG. 24 is a table showing a comparison of the long-term stability data for the BCA101 DS presented in FIG. 22 and the DP presented in FIG. 23.

FIG. 25 is a series of dot graphs showing the percent LMWP, percent monomer, and percent HMWP of BCA101 DS formulated in a liquid formulation comprising 25 mg/ml BCA101, 8.0% w/v sucrose, 0.02% w/v polysorbate 20, and 10 mM citrate phosphate buffer (pH 6.0) over the course of 24 months stored in 5 mL Celsius bags at −20° C. Samples were analyzed at the initial time point, 1 month, 2 months, 3 months, 6 months, 12 months, 18 months, and 24 months.

FIG. 26 is a series of dot graphs showing the percent LMWP, percent monomer, and percent HMWP of BCA101 DP formulated in a liquid formulation comprising 25 mg/ml BCA101, 8.0% w/v sucrose, 0.02% w/v polysorbate 20, and 10 mM citrate phosphate buffer (pH 6.0) over the course of 24 months stored in 5 mL Celsius bags at −20° C. Samples were analyzed at the initial time point, 1 month, 2 months, 3 months, 6 months, 12 months, 18 months, and 24 months.

FIG. 27 is a table showing a comparison of the long-term stability data for the BCA101 DS presented in FIG. 25 and the DP presented in FIG. 26.

FIG. 28 is a diagram showing an exemplary manufacturing process of the present disclosure to manufacture a BCA101 formulation described herein, e.g., a formulation comprising 25 mg/ml BCA101, 8.0% w/v sucrose, 0.02% w/v polysorbate 20, and 10 mM citrate phosphate buffer (pH 6.0).

FIG. 29 is a line graph showing the trend of pH of BCA100 drug substance (DS) stability data at −20±5° C. storage over 24 months.

FIG. 30 is a line graph showing the trend of osmolality of BCA100 drug substance (DS) stability data at −20±5° C. storage over 24 months.

FIG. 31 is a line graph showing the trend of protein concentration of BCA100 drug substance (DS) stability data at −20±5° C. storage over 24 months.

FIG. 32 is a line graph showing the trend of the percent of high molecule weight protein of BCA100 drug substance (DS) stability data at −20±5° C. storage over 24 months.

FIG. 33 is a line graph showing the trend of the percent of monomeric protein of BCA100 drug substance (DS) stability data at −20±5° C. storage over 24 months.

FIG. 34 is a line graph showing the trend of the percent of low molecule weight protein of BCA100 drug substance (DS) stability data at −20±5° C. storage over 24 months.

FIG. 35 is a line graph showing the trend of the percent of total protein pre-peak of BCA100 drug substance (DS) stability data at −20±5° C. storage over 24 months.

FIG. 36 is a line graph showing the trend of the percent of total protein post peak of BCA100 drug substance (DS) stability data at −20±5° C. storage over 24 months.

FIG. 37 is a line graph showing the trend of the percent of total protein at main peak of BCA100 drug substance (DS) stability data at −20±5° C. storage over 24 months.

FIG. 38 is a line graph showing the trend of the relative potency of BCA100 drug substance (DS) stability data at −20±5° C. storage over 24 months, as measured by bifunctional ELISA.

FIG. 39 is a line graph showing the trend of the relative potency of BCA100 drug substance (DS) stability data at −20±5° C. storage over 24 months, as measured by inhibition of proliferation (TOP) assay.

FIG. 40 is a line graph showing the trend of pH of BCA100 drug product (DP) stability data at 5±3° C. storage over 24 months.

FIG. 41 is a line graph showing the trend of osmolality of BCA100 drug product (DP) stability data at 5±3° C. storage over 24 months.

FIG. 42 is a line graph showing the trend of protein concentration of BCA100 drug product (DP) stability data at 5±3° C. C storage over 24 months.

FIG. 43 is a line graph showing the trend of extractable volume of BCA100 drug product (DP) stability data at 5±3° C. storage over 24 months.

FIG. 44 is a line graph showing the trend of the percent of high molecule weight protein of BCA100 drug product (DP) stability data at 5±3° C. storage over 24 months.

FIG. 45 is a line graph showing the trend of the percent of monomeric protein of BCA100 drug product (DP) stability data at 5±3° C. storage over 24 months.

FIG. 46 is a line graph showing the trend of the percent of low molecule weight protein of BCA100 drug product (DP) stability data at 5±3° C. storage over 24 months.

FIG. 47 is a line graph showing the trend of the percent of total protein pre-peak of BCA100 drug product (DP) stability data at 5±3° C. storage over 24 months.

FIG. 48 is a line graph showing the trend of the percent of total protein post peak of BCA100 drug product (DP) stability data at 5±3° C. storage over 24 months.

FIG. 49 is a line graph showing the trend of the percent of total protein at main peak of BCA100 drug product (DP) stability data at 5±3° C. storage over 24 months.

FIG. 50 is a line graph showing the trend of the relative potency of BCA100 drug product (DP) stability data at 5±3° C. storage over 24 months, as measured by bifunctional ELISA.

FIG. 51 is a line graph showing the trend of the relative potency of BCA100 drug product (DP) stability data at 5±3° C. storage over 24 months, as measured by inhibition of proliferation (TOP) assay.

FIG. 52 is an iCE electropherograms of BCA101 DS batch BL.14.0901/R/17/021/F DS (top panel) and BCA101 DS GF19000040 (bottom panel). The left and right pI markers correspond to 4.05 and 8.4 respectively.

FIG. 53 shows the intact mass spectra showing intact mass of BCA101 DS batch BL.14.0901/R/17/021/F DS (top panel) and BCA101 DS GF19000040 (bottom panel) using MALDI-TOF.

FIG. 54 shows a UV chromatogram of peptides generated from tryptic digest of BCA101 DS batch BL.14.0901/R/17/021/F DS (top panel) and BCA101 DS batch GF19000040 (bottom panel).

FIG. 55 shows the MS 2 tandem spectra for N-terminus of heavy chain of BCA101 DS batch GF19000040.

FIG. 56 shows the MS 2 tandem spectra for C-terminus of heavy chain of BCA101 DS batch GF19000040.

FIG. 57 shows the MS 2 tandem spectra for N-terminus of light chain of BCA101 DS batch GF19000040.

FIG. 58 shows the MS 2 tandem spectra for C-terminus of linker of BCA101 DS batch GF19000040.

FIG. 59 shows the MS spectrum for C-terminus of TGFβRII chain of BCA101 DS batch GF19000040.

FIG. 60 shows the far-UV CD spectra overlay of BCA101 DS batch GF19000040 along BL.14.0901/R/17/021/F DS.

FIG. 61 shows the near-UV CD spectra overlay of BCA101 DS batch GF19000040 along BL.14.0901/R/17/021/F DS.

FIG. 62 shows the UV chromatogram profile of tryptic non-reduced peptides of BCA101 DS batch GF19000040 along BL.14.0901/R/17/021/F DS.

FIG. 63 shows the overlay of NP-HPLC profile of N-glycans of BCA101 DS batch GF19000040 along BL.14.0901/R/17/021/F DS. The N-glycans printed here were identified by MS. Identification of ‘other species’ is ongoing.

FIG. 64 shows the overlay of RP-HPLC chromatogram for sialic acid estimation of BCA101 DS batch GF19000040 along with NGNA, NANA standards and corresponding buffer blank.

### INCORPORATION BY REFERENCE

All publications, patents, and patent applications mentioned in this specification are incorporated by reference herein to the same extent as if each individual publication, patent, or patent application was specifically and individually indicated to be incorporated by reference.

### DETAILED DESCRIPTION

### Overview

The present disclosure provides, inter alia, pharmaceutical formulations for bifunctional fusion proteins described herein, that enable long term storage of the preparation without untenable protein instability (e.g., degradation) or loss of bifunctional activity. In some embodiments, one functional aspect of the fusion protein (e.g., a targeting moiety) as an acidic pI, while the second functional aspect (e.g., an immunomodulatory domain) has a basic pI. In some embodiments, the hEGFR fusion protein comprises a targeting moiety that specifically binds hEGFR and an immunomodulatory moiety that comprises an amino acid sequence of the extracellular domain of hTGFβRII. The pharmaceutical compositions disclosed herein can be particularly useful in the treatment of hEGFR driven cancers.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise.

It must be noted that, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and/or “consisting essentially of” are also provided.

The section headings used herein are for organizational purposes only and are not to be construed as limiting the subject matter described.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “subject” and “patient” are used interchangeably herein and include any human or nonhuman animal. The term “nonhuman animal” includes, but is not limited to, vertebrates such as nonhuman primates, sheep, dogs, and rodents such as mice, rats and guinea pigs. In some embodiments, the subject is a human.

As used herein, the term “administering” refers to the physical introduction of a therapeutic agent (or a precursor of the therapeutic agent that is metabolized or altered within the body of the subject to produce the therapeutic agent in vivo) to a subject, using any of the various methods and delivery systems known to those skilled in the art. Exemplary routes of include intravenous, intramuscular, subcutaneous, intraperitoneal, spinal or other parenteral routes of administration, for example by injection or infusion. The term “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent may be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “cancer” and “tumor” are used interchangeably herein and refer to a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

A “therapeutically effective amount” or “therapeutically effective dose” of a therapeutic agent is any amount of the therapeutic agent that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “antibody” is used herein in the broadest sense and encompasses fully assembled antibodies; functional antibody fragments and functional variants thereof that can bind antigen (e.g., Fab, F(ab′)2, Fv, single chain variable fragment (scFv), single domain antibodies (e.g., VHH), diabodies, antibody chimeras, hybrid antibodies, bispecific antibodies, and the like); and non-antibody fragments that bind antigen (e.g., recombinant fibronectin domains) and recombinant polypeptides comprising the forgoing. Unless otherwise specified, references to the numbering of specific amino acid residue positions in an antibody are according to the EU numbering system, as described in Kabat et al., U.S. Dept. of Health and Human Services, Sequences of Proteins of Immunological Interest (1983) (“Kabat”), the full contents of which are incorporated by reference herein.

As used herein, the term “variable region” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions and three complementarity determining regions.

As used herein, the term “complementarity determining region” refers to each of the regions of an antibody variable domain which are hypervariable in sequence and form structurally defined loops (“hypervariable loops”). Generally, native four-chain antibodies comprise six CDRs; three in the VH (H1, H2, H3), and three in the VL (L1, L2, L3). The CDRs have been described by Kabat et al., U.S. Dept. of Health and Human Services, Sequences of Proteins of Immunological Interest (1983) (“Kabat”) and by Chothia et al., J Mol Biol 196:901-917 (1987), where the definitions include overlapping or subsets of amino acid residues when compared against each other. Nevertheless, application of either definition to refer to a CDR of an antibody is intended to be within the scope of the term as defined and used herein. Those skilled in the art can routinely determine which residues comprise a particular CDR given the variable region amino acid sequence of the antibody. Unless otherwise specified, CDRs are defined according to the Kabat system.

The term “fusion protein” and grammatical equivalents as used herein refers to a protein that comprises an amino acid sequence derived from at least two separate proteins. The amino acid sequence of the at least two separate proteins can be directly connected through a peptide bond; or can be operably connected through an amino acid linker. Therefore, the term fusion protein encompasses embodiments, wherein the amino acid sequence of e.g., Protein A is directly connected to the amino acid sequence of Protein B through a peptide bond (Protein A—Protein B), and embodiments, wherein the amino acid sequence of e.g., Protein A is operably connected to the amino acid sequence of Protein B through an amino acid linker (Protein A—linker—Protein B).

The term “fuse” and grammatical equivalents thereof as used herein refers to the operable connection of an amino acid sequence derived from one protein to the amino acid sequence derived from different protein. The term fuse encompasses both a direct connection of the two amino acid sequences through a peptide bond, and the indirect connection through an amino acid linker.

As used herein, the term “modification,” with reference to a nucleic acid sequence, refers to a nucleic acid sequence that comprises at least one substitution, addition, or deletion of nucleotide compared to a reference nucleic acid sequence. As used herein, the term “modification,” with reference to an amino acid sequence refers to an amino acid sequence that comprises at least one substitution, addition, or deletion of an amino acid residue compared to a reference nucleic acid sequence. Naturally occurring amino acid derivatives are not considered modified amino acids for purposes of determining percent identity of two amino acid sequences. For example, a naturally occurring modification of a glutamate amino acid residue to a pyroglutamate amino acid residue would not be considered an amino acid modification for purposes of determining percent identity of two amino acid sequences. Further, for example, a naturally occurring modification of a glutamate amino acid residue to a pyroglutamate amino acid residue would not be considered an amino acid “modification” as defined herein. Modifications can include the inclusion of non-naturally occurring amino acid residues.

The term “identical” or “percent identity” with reference to a nucleic acid sequence or amino acid sequence refers to at least two nucleic acid or at least two amino acid sequences or subsequences that have a specified percentage of nucleotides or amino acids, respectively, that are the same, when compared and aligned for maximum correspondence, as measured using a sequence comparison algorithm or by visual inspection. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information. The percent identity between two sequences can be determined using techniques similar to those described above, with or without allowing gaps. In calculating percent identity, typically only exact matches are counted. As described above, the percent identity is based on the amino acid matches between the smaller of two proteins.

A “stable” pharmaceutical composition is one in which the protein therein essentially retains its physical stability and/or chemical stability and/or biological activity upon processing (e.g., ultrafiltration, diafiltration, other filtering steps, vial filling), transportation, and/or storage of the drug substance and/or drug product containing a fusion protein described herein. Together, the physical, chemical and biological stability of the protein in a formulation embody the “stability” of the protein formulation, e.g., the fusion protein formulation, which is specific to the conditions under which the formulated drug product (DP) is stored.

A protein retains its “physical stability” in a pharmaceutical composition if it shows minimal signs of changes to the secondary and/or tertiary structure (i.e., intrinsic structure), or aggregation, and/or precipitation and/or denaturation upon visual examination of color and/or clarity, or as measured by UV light scattering or by size exclusion high performance liquid chromatography, or other suitable methods. Physical instability of a protein, i.e., loss of physical stability, can be caused by oligomerization resulting in dimer and higher order aggregates, subvisible, and visible particle formation, and precipitation. The degree of physical degradation can be ascertained using varying techniques depending on the type of degradant of interest. Dimers and higher order soluble aggregates can be quantified using size exclusion chromatography, while subvisible particles may be quantified using light scattering, light obscuration or other suitable techniques.

A protein retains its “chemical stability” in a pharmaceutical composition, if the chemical stability at a given time is such that covalent bonds are not made or broken, resulting in changes to the primary structure of the protein component, e.g., a fusion protein described herein. Changes to the primary structure may result in modifications of the secondary and/or tertiary and/or quaternary structure of the protein and may result in formation of aggregates or reversal of aggregates already formed. Typical chemical modifications can include isomerization, deamidation, N-terminal cyclization, backbone hydrolysis, methionine oxidation, tryptophan oxidation, histidine oxidation, beta-elimination, disulfide formation, disulfide scrambling, disulfide cleavage, and other changes resulting in changes to the primary structure including D-amino acid formation. Chemical instability, i.e., loss of chemical stability, may be interrogated by a variety of techniques including ion-exchange chromatography, capillary isoelectric focusing, analysis of peptide digests and multiple types of mass spectrometric techniques. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by charge-based methods, such as, but not limited to, ion-exchange chromatography, capillary isoelectric focusing, or peptide mapping.

Loss of physical and/or chemical stability may result in changes to biological activity as either an increase or decrease of a biological activity of interest, depending on the modification and the protein being modified. A protein retains its “biological activity” in a pharmaceutical compositions, if the biological activity of the protein at a given time is within at least 30% of the biological activity exhibited at the time the pharmaceutical formulation was prepared. Activity is considered decreased if the activity is less than 70% of its starting value. Biological assays may include both in vivo and in vitro based assays such as ligand binding, potency, cell proliferation or other surrogate measure of its biopharmaceutical activity. As an example, biological activity of BCA101 described herein can be estimated using an in ELISA that measures binding capability to both hEGFR and hTGFβ. Briefly, to carry out the bifunctional ELISA, recombinant hEGFR Fc coated plates were blocked and subsequently incubated with BCA101 for about 1 hour, followed by incubation with recombinant hTGFβ1. hTGFβ1 bound to hTGFβRII ECD moiety of BCA101 was then detected with biotinylated anti-hTGFβ1 antibody followed by streptavidin-HRP. Thereby, the signal will be obtained only when both arms are intact.

### Pharmaceutical Compositions

In certain aspects, described herein are pharmaceutical compositions (e.g., liquid pharmaceutical compositions) that comprise a fusion protein (e.g., a fusion protein described herein), a buffer, and a tonifying agent, with a preselected pH. In some embodiments, the pharmaceutical compositions comprise one or more further agents, including e.g., a surfactant. In some embodiments, the pharmaceutical composition is a liquid or powder form. In some embodiments, the pharmaceutical composition is a liquid form. In some embodiments, the pharmaceutical composition is specifically suitable for intravenous administration to a subject (e.g., a human subject).

### Buffers

In some embodiments, the buffer is a citrate phosphate buffer, citrate buffer, succinate buffer, or histidine buffer. In some embodiments, the buffer is a citrate phosphate buffer. In some embodiments, the buffer is a citrate buffer. In some embodiments, the buffer is a succinate buffer. In some embodiments, the buffer is a histidine buffer.

In some embodiments, the concentration of the buffer is from about 5 mM to about 40 mM, 5 mM to about 35 mM, 5 mM to about 30 mM, 5 mM to about 25 mM, 5 mM to about 20 mM, 5 mM to about 15 mM, 5 mM to about 10 mM, or 10 mM to about 30 mM. In some embodiments, the concentration of the buffer is from about 5 mM to about 15 mM. In some embodiments, the concentration of the buffer is about 5 mM, 10 mM, 15 mM, 20 mM, 25 mM, or 30 mM. In some embodiments, the concentration of the buffer is about 10 mM.

In certain embodiments, the buffer is a citrate phosphate buffer present in a concentration from about 5 mM to about 30 mM, 5 mM to about 25 mM, 5 mM to about 20 mM, 5 mM to about 15 mM, 5 mM to about 10 mM, or 10 mM to about 30 mM. In some embodiments, the buffer is a citrate phosphate buffer present in a concentration of about 5 mM to about 15 mM. In some embodiments, the buffer is a citrate phosphate buffer present in a concentration of about 5 mM, 10 mM, 15 mM, 20 mM, 25 mM, or 30 mM. In some embodiments, the buffer is a citrate phosphate buffer present in a concentration of about 10 mM. In some embodiments, the pharmaceutical composition has a pH from about 6.0 to 6.5. In some embodiments, the pharmaceutical composition has a pH of about 6.0.

pH

In some embodiments, the pharmaceutical composition has a pH from about 5.0 to about 8.0. In some embodiments, the pharmaceutical composition has a pH from about 5.5 to about 7.0, 6.0 to about 7.0, 5.5 to about 6.5, 5.5 to about 6.0, or 6.0 to about 6.5. In some embodiments, the pharmaceutical composition has a pH from about 6.0 to about 6.5. In some embodiments, the pharmaceutical composition has a pH of about 5.5, 6.0, 6.5. or 7.0. In some embodiments, the pharmaceutical composition has a pH of about 6.0. In some embodiments, the pharmaceutical composition has a pH of about 6.5.

### Tonifying Agents

In some embodiments, the pharmaceutical composition comprises a tonifying agent such that the formulation has a final osmolality from about 200-400 mOsmol/kg. In some embodiments, the pharmaceutical composition comprises a tonifying agent such that the formulation has a final osmolality from about 250-350 mOsmol/kg. In some embodiments, the pharmaceutical composition comprises a tonifying agent such that the formulation has a final osmolality of about 300 mOsmol/kg.

In some embodiments, the tonifying agent comprises sucrose, trehalose, sorbitol, mannitol, or glycerol. In some embodiments, the tonifying agent is sucrose. In some embodiments, the tonifying agent is trehalose. In some embodiments, the tonifying agent is present at a concentration from about 5% w/v to about 10% w/v, 6% w/v to about 10% w/v, 7% w/v to about 10% w/v, 8% w/v to about 10% w/v, 5% w/v to about 9% w/v, 5% w/v to about 8% w/v, 6% w/v to about 9% w/v, 6% w/v to about 8% w/v, 7% w/v to about 9% w/v, or 7% w/v to about 8% w/v. In some embodiments, the tonifying agent is present at a concentration from about 5% w/v to about 8% w/v. In some embodiments, the tonifying agent is present at a concentration of about 5% w/v, 6% w/v, 7% w/v, 8% w/v, 9% w/v, or 10% w/v. In some embodiments, the tonifying agent is present at a concentration of about 8% w/v.

In some embodiments, the tonifying agent is sucrose at a concentration such that the formulation has a final osmolality of about 300 mOsmol/kg. In some embodiments, the tonifying agent comprises sucrose at a concentration from about 5% w/v to about 10% w/v, 6% w/v to about 10% w/v, 7% w/v to about 10% w/v, 8% w/v to about 10% w/v, 5% w/v to about 9% w/v, 5% w/v to about 8% w/v, 6% w/v to about 9% w/v, 6% w/v to about 8% w/v, 7% w/v to about 9% w/v, or 7% w/v to about 8% w/v. In some embodiments, the tonifying agent comprises sucrose at a concentration from about 5% w/v to about 8% w/v. In some embodiments, the tonifying agent is present at a concentration of about 5% w/v, 6% w/v, 7% w/v, 8% w/v, 9% w/v, or 10% w/v. In some embodiments, the tonifying agent is sucrose at a concentration of about 8% w/v.

### Surfactants

In some embodiments, the pharmaceutical composition comprises a surfactant. In some embodiments, the surfactant is a non-ionic surfactant. In some embodiments, the surfactant is a polysorbate, a polyethylene glycol dodecyl ether, a poloxamer, 4-(1,1,3,3-Tetramethylbutyl)phenyl-polyethylene glycol, an alkylsaccharide and an alkylglycoside, Brij®35 (i.e., polyethylene glycol dodecyl ether), a poloxamer (i.e., Polyethylene-Polypropylene Glycol; Polyoxyethylene-Polyoxypropylene Block Copolymer; Poly(Ethylene oxide-co-Polypropylene oxide)) such as Poloxamer 188 (i.e., Pluronic F68), or Triton™ X-100 (i.e., 4-(1,1,3,3-Tetramethylbutyl)phenyl-polyethylene glycol)). Exemplary polysorbates includes, but are not limited to, polysorbate 20, polysorbate 40, polysorbate 60, or polysorbate 80. In some embodiments, the surfactant is polysorbate 20.

In some embodiments, the concentration of the surfactant is from about 0.005-0.1% w/v, 0.01-0.1% w/v, 0.02-0.1% w/v, 0.01-0.9% w/v, 0.01-0.8% w/v, 0.01-0.7% w/v, 0.01-0.6% w/v, 0.01-0.5% w/v, 0.01-0.4% w/v, 0.01-0.3% w/v, 0.01-0.2% w/v, 0.01-0.1% w/v, 0.02-0.9% w/v, 0.02-0.8% w/v, 0.02-0.7% w/v, 0.02-0.6% w/v, 0.02-0.5% w/v, 0.02-0.4% w/v, 0.02-0.3% w/v, 0.02-0.2% w/v, 0.02-0.1% w/v, 0.005-0.9% w/v, 0.005-0.8% w/v, 0.005-0.7% w/v, 0.005-0.6% w/v, 0.005-0.5% w/v, 0.005-0.4% w/v, 0.005-0.3% w/v, 0.005-0.2% w/v, or 0.005-0.1% w/v. In some embodiments, the concentration of the surfactant is about 0.01% w/v, 0.02% w/v, 0.03% w/v, 0.04% w/v, 0.05% w/v, 0.06% w/v, 0.07% w/v, 0.08% w/v, 0.09% w/v, or 0.1% w/v. In some embodiments, the concentration of the surfactant is about 0.02% w/v.

In some embodiments, the surfactant is polysorbate 20 at a concentration from about 0.005-0.1% w/v, 0.01-0.1% w/v, 0.02-0.1% w/v, 0.01-0.9% w/v, 0.01-0.8% w/v, 0.01-0.7% w/v, 0.01-0.6% w/v, 0.01-0.5% w/v, 0.01-0.4% w/v, 0.01-0.3% w/v, 0.01-0.2% w/v, 0.01-0.1% w/v, 0.02-0.9% w/v, 0.02-0.8% w/v, 0.02-0.7% w/v, 0.02-0.6% w/v, 0.02-0.5% w/v, 0.02-0.4% w/v, 0.02-0.3% w/v, 0.02-0.2% w/v, 0.02-0.1% w/v, 0.005-0.9% w/v, 0.005-0.8% w/v, 0.005-0.7% w/v, 0.005-0.6% w/v, 0.005-0.5% w/v, 0.005-0.4% w/v, 0.005-0.3% w/v, 0.005-0.2% w/v, or 0.005-0.1% w/v. In some embodiments, the surfactant is polysorbate 20 at a concentration of about 0.01% w/v, 0.02% w/v, 0.03% w/v, 0.04% w/v, 0.05% w/v, 0.06% w/v, 0.07% w/v, 0.08% w/v, 0.09% w/v, or 0.1% w/v. In some embodiments, the surfactant is polysorbate 20 at a concentration of about 0.02% w/v.

### Osmolality

In some embodiments, pharmaceutical composition has an osmolality from about 100 mOsmol/kg to about 400 mOsmol/kg, about 150 mOsmol/kg to about 400 mOsmol/kg, 150 mOsmol/kg to about 350 mOsmol/kg, 150 mOsmol/kg to about 300 mOsmol/kg, 200 mOsmol/kg to about 400 mOsmol/kg, 250 mOsmol/kg to about 400 mOsmol/kg, 300 mOsmol/kg to about 400 mOsmol/kg, 300 mOsmol/kg to about 350 mOsmol/kg, 250 mOsmol/kg to about 350 mOsmol/kg, or 250 mOsmol/kg to about 300 mOsmol/kg. In some embodiments, the pharmaceutical composition has an osmolality from about 250 mOsmol/kg to about 350 mOsmol/kg. In some embodiments, the pharmaceutical composition has an osmolality of about 250 mOsmol/kg, 300 mOsmol/kg, or 350 mOsmol/kg. In some embodiments, the pharmaceutical composition has an osmolality of about 300 mOsmol/kg.

### Exemplary Functional Properties

### Stability and Storage

The pharmaceutical compositions can be stored in any suitable container known to the skilled artisan, e.g., a bag (e.g., Celsius® bags, Flexboy® bags) or glass vials (e.g., USP 10R glass vials). Containers for proper storage at variant temperatures (e.g., −80° C., −20° C., 2-8° C.) are known to the skilled artisan and can be selected accordingly. In some embodiments, when stability is measured at −20° C. the pharmaceutical compositions are stored in a Celsius® bag or Flexboy® bag. In some embodiments, when stability is measured at 2-8° C. the pharmaceutical compositions are stored in glass vials.

In some embodiments, the pharmaceutical composition exhibits increased stability for at least 3, 6, 12, 18, 24, or 36 months when refrigerated or frozen compared to a reference pharmaceutical composition. In some embodiments, the pharmaceutical composition exhibits increased chemical stability for at least 3, 6, 12, 18, 24, or 36 months when refrigerated or frozen compared to a reference pharmaceutical composition. In some embodiments, the pharmaceutical composition exhibits increased physical stability for at least 3, 6, 12, 18, 24, or 36 months when refrigerated or frozen compared to a reference pharmaceutical composition.

In some embodiments, the pharmaceutical composition is stable for at least 3, 6, 12, 18, 24, or 36 months when refrigerated or frozen. In some embodiments, the pharmaceutical composition is stable for at least 3, 6, 12, 18, 24, or 36 months when stored at −80° C. In some embodiments, the pharmaceutical composition is stable for at least 3, 6, 12, 18, 24, or 36 months when refrigerated or frozen. In some embodiments, the pharmaceutical composition is stable for at least 3, 6, 12, 18, 24, or 36 months when stored at −20° C. In some embodiments, the pharmaceutical composition is stable for at least 3, 6, 12, 18, 24, or 36 months when stored at 2-8° C.

In some embodiments, the pharmaceutical composition is chemically stable for at least 3, 6, 12, 18, 24, or 36 months when refrigerated or frozen. In some embodiments, the pharmaceutical composition is chemically stable for at least 3, 6, 12, 18, 24, or 36 months when stored at −80° C. In some embodiments, the pharmaceutical composition is chemically stable for at least 3, 6, 12, 18, 24, or 36 months when refrigerated or frozen. In some embodiments, the pharmaceutical composition is chemically stable for at least 3, 6, 12, 18, 24, or 36 months when stored at −20° C. In some embodiments, the pharmaceutical composition is chemically stable for at least 3, 6, 12, 18, 24, or 36 months when stored at 2-8° C.

In some embodiments, the pharmaceutical composition is physically stable for at least 3, 6, 12, 18, 24, or 36 months when refrigerated or frozen. In some embodiments, the pharmaceutical composition is physically stable for at least 3, 6, 12, 18, 24, or 36 months when stored at −80° C. In some embodiments, the pharmaceutical composition is physically stable for at least 3, 6, 12, 18, 24, or 36 months when stored at −20° C. In some embodiments, the pharmaceutical composition is physically stable for at least 3, 6, 12, 18, 24, or 36 months when stored at 2-8° C.

In some embodiments, the pharmaceutical composition is chemically and physically stable for at least 3, 6, 12, 18, 24, or 36 months when refrigerated or frozen. In some embodiments, the pharmaceutical composition is chemically and physically stable for at least 3, 6, 12, 18, 24, or 36 months when stored at −80° C. In some embodiments, the pharmaceutical composition is chemically and physically stable for at least 3, 6, 12, 18, 24, or 36 months when stored at −20° C. In some embodiments, the pharmaceutical composition is chemically and physically stable for at least 3, 6, 12, 18, 24, or 36 months when stored at 2-8° C.

In some embodiments, the pharmaceutical composition is stable through at least 1, 2, 3, 4 or 5 freeze thaw cycles, wherein the freeze thaw cycle comprises 48-hour freeze cycle at −80° C. or −20° C.; and the thaw cycle comprises a 4 hour thaw 25° C. in incubator. In some embodiments, the pharmaceutical composition is chemically stable through at least 1, 2, 3, 4 or 5 freeze thaw cycles, wherein the freeze thaw cycle comprises 48-hour freeze cycle at −80° C. or −20° C.; and the thaw cycle comprises a 4 hour thaw 25° C. in incubator. In some embodiments, the pharmaceutical composition is physically stable through at least 1, 2, 3, 4 or 5 freeze thaw cycles, wherein the freeze thaw cycle comprises 48-hour freeze cycle at −80° C. or −20° C.; and the thaw cycle comprises a 4 hour thaw 25° C. in incubator. In some embodiments, the pharmaceutical composition is chemically and physically stable through at least 1, 2, 3, 4 or 5 freeze thaw cycles, wherein the freeze thaw cycle comprises 48-hour freeze cycle at −80° C. or −20° C.; and the thaw cycle comprises a 4 hour thaw 25° C. in incubator.

In some embodiments, the concentration of said fusion protein in said liquid pharmaceutical composition is substantially the same for at least 3, 6, 12, 18, 24, or 36 months when stored at −80° C. In some embodiments, the concentration of said fusion protein in said liquid pharmaceutical composition is substantially the same for at least 3, 6, 12, 18, 24, or 36 months when stored at −20° C. In some embodiments, the concentration of said fusion protein in said liquid pharmaceutical composition is substantially the same for at least 3, 6, 12, 18, 24, or 36 months when stored at 2-8° C.

In some embodiments, the concentration of said fusion protein in said liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 3, 6, 12, 18, 24, or 36 months at −80° C. In some embodiments, the concentration of said fusion protein in said liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 3, 6, 12, 18, 24, or 36 months at −20° C. In some embodiments, the concentration of said fusion protein in said liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 3, 6, 12, 18, 24, or 36 months at 2-8° C.

In some embodiments, the pharmaceutical composition has a shelf life of at least 12 months, 24 months, 36 months, or 48 months, when stored at −80° C. In some embodiments, the pharmaceutical composition has a shelf life of at least 12 months, 24 months, 36 months, or 48 months, when stored at −20° C. In some embodiments, the pharmaceutical composition has a shelf life of at least 12 months, 24 months, 36 months, or 48 months, when stored at 2-8° C.

In some embodiments, pharmaceutical composition comprises less than about 20%, 10%, 9%, 8%, 7%, 6%, 5%, 4%, 3%, 2%, or 1% of said fusion protein in aggregate form. In some embodiments, pharmaceutical composition comprises less than about 10%, 9%, 8%, 7%, 6%, 5%, 4%, 3%, 2%, or 1% of said fusion protein in aggregate form. In some embodiments, pharmaceutical composition comprises less than about 5%, 4%, 3%, 2%, or 1% of said fusion protein in aggregate form. In some embodiments, pharmaceutical composition comprises less than about 5% of said fusion protein in aggregate form. In some embodiments, pharmaceutical composition comprises less than about 30%, 20%, 10%, 9%, 8%, 7%, 6%, 5%, 4%, 3%, 2%, or 1% of said fusion protein in aggregate form after storage at −20° C. for at least 12 months, 24 months, or 36 months. In some embodiments, pharmaceutical composition comprises less than about 30%, 20%, 10%, 9%, 8%, 7%, 6%, 5%, 4%, 3%, 2%, or 1% of said fusion protein in aggregate form after storage at 2-8° C. for at least 12 months, 24 months, or 36 months.

In some embodiments, the pharmaceutical composition comprises no more than 1%, 2%, 3%, 4%, 5%, 6%, 7%, 8%, 9%, 10%, or 20% fusion protein in aggregate form. In some embodiments, the pharmaceutical composition comprises no more than 1%, 2%, 3%, 4%, 5%, 6%, 7%, 8%, 9%, or 10% fusion protein in aggregate form. In some embodiments, the pharmaceutical composition comprises no more than 1%, 2%, 3%, 4%, or 5% fusion protein in aggregate form. In some embodiments, the pharmaceutical composition comprises no more than 5% fusion protein in aggregate form. In some embodiments, the pharmaceutical composition comprises no more than 1%, 2%, 3%, 4%, 5%, 6%, 7%, 8%, 9%, 10%, or 20% fusion protein in aggregate form after storage at −80° C. for at least 12 months, 24 months, or 36 months. In some embodiments, the pharmaceutical composition comprises no more than 1%, 2%, 3%, 4%, 5%, 6%, 7%, 8%, 9%, or 10% fusion protein in aggregate form after storage at −80° C. for at least 12 months, 24 months, or 36 months. In some embodiments, the pharmaceutical composition comprises no more than 1%, 2%, 3%, 4%, 5% fusion protein in aggregate form after storage at −80° C. for at least 12 months, 24 months, or 36 months. In some embodiments, the pharmaceutical composition comprises no more than 5% fusion protein in aggregate form after storage at −80° C. for at least 12 months, 24 months, or 36 months. In some embodiments, the pharmaceutical composition comprises no more than 1%, 2%, 3%, 4%, 5%, 6%, 7%, 8%, 9%, 10%, or 20% fusion protein in aggregate form. In some embodiments, the pharmaceutical composition comprises no more than 1%, 2%, 3%, 4%, 5%, 6%, 7%, 8%, 9%, 10%, 20%, or 30% fusion protein in aggregate form after storage at −20° C. for at least 12 months, 24 months, or 36 months. In some embodiments, the pharmaceutical composition comprises no more than 1%, 2%, 3%, 4%, 5%, 6%, 7%, 8%, 9%, or 10% fusion protein in aggregate form. In some embodiments, the pharmaceutical composition comprises no more than 1%, 2%, 3%, 4%, 5%, 6%, 7%, 8%, 9%, 10%, 20%, or 30% fusion protein in aggregate form after storage at −20° C. for at least 12 months, 24 months, or 36 months. In some embodiments, the pharmaceutical composition comprises no more than 1%, 2%, 3%, 4%, 5%, 6%, 7%, 8%, 9%, 10%, 20%, or 30% fusion protein in aggregate form. In some embodiments, the pharmaceutical composition comprises no more than 1%, 2%, 3%, 4%, or 5% fusion protein in aggregate form after storage at −20° C. for at least 12 months, 24 months, or 36 months. In some embodiments, the pharmaceutical composition comprises no more than 5% fusion protein in aggregate form. In some embodiments, the pharmaceutical composition comprises no more than 1%, 2%, 3%, 4%, 5%, 6%, 7%, 8%, 9%, 10%, 20%, or 30% fusion protein in aggregate form after storage at −20° C. for at least 12 months, 24 months, or 36 months. In some embodiments, the pharmaceutical composition comprises no more than 1%, 2%, 3%, 4%, 5%, 6%, 7%, 8%, 9%, 10%, or 20% fusion protein in aggregate form after storage at 2-8° C. for at least 12 months, 24 months, or 36 months. In some embodiments, the pharmaceutical composition comprises no more than 1%, 2%, 3%, 4%, 5%, 6%, 7%, 8%, 9%, or 10% fusion protein in aggregate form after storage at 2-8° C. for at least 12 months, 24 months, or 36 months. In some embodiments, the pharmaceutical composition comprises no more than 1%, 2%, 3%, 4%, 5% fusion protein in aggregate form after storage at 2-8° C. for at least 12 months, 24 months, or 36 months. In some embodiments, the pharmaceutical composition comprises no more than 5% fusion protein in aggregate form after storage at 2-8° C. for at least 12 months, 24 months, or 36 months. Aggregation can be measured by any suitable method known in the art, including as described in Example 1 of the instant disclosure. Aggregation can be evaluated for example by size exclusion chromatography (SEC).

Stability can be measured by any assay known to the skilled artisan, including those described in Example 1 of the instant disclosure. Various analytical techniques for measuring protein stability are reviewed, e.g., in Wang, W. (1999), Instability, stabilization and formulation of liquid protein pharmaceuticals, Int J Pharm 185: 129-188. Stability can be measured at a selected temperature for a selected time period (e.g., 1 week, 3 months, 6 months, 9 months, 12 months, 18 months, 24 months, or 36 months).

### Bifunctionality of Fusion Proteins

The fusion proteins described herein have 2 distinct functions: 1) specifically bind hEGFR and 2) specifically bind hTGFβ. In some embodiments, the fusion protein retains bifunctional activity for at least 3, 6, 12, 18, 24, or 36 months when refrigerated or frozen. In some embodiments, the fusion protein retains bifunctional activity for at least 3, 6, 12, 18, 24, or 36 months when stored at −20° C. In some embodiments, the fusion protein retains bifunctional activity for at least 3, 6, 12, 18, 24, or 36 months when stored at 2-8° C.

In some embodiments, the fusion proteins described herein retain at least 95%, 96%, 97%, 98%, 99%, or 100% of their hEGFR binding activity (e.g., as measured by ELISA). In some embodiments, the fusion proteins described herein retain at 95%, 96%, 97%, 98%, 99%, or 100% of their hTGFβ binding activity (e.g., as measured by ELISA). In some embodiments, the fusion proteins described herein retain at least 95%, 96%, 97%, 98%, 99%, or 100% of their hEGFR binding activity (e.g., as measured by ELISA); and retain at least 96%, 97%, 98%, 99%, or 100% of their hTGFβ binding activity (e.g., as measured by ELISA).

In some embodiments, the fusion proteins described herein lose less than, 5%, 4%, 3%, 2%, 1%, or 0.5% of their hEGFR binding activity (e.g., as measured by ELISA). In some embodiments, the fusion proteins described herein lose less than 5%, 4%, 3%, 2%, 1%, or 0.5% of their hTGFβ binding activity (e.g., as measured by ELISA). In some embodiments, the fusion proteins described herein lose less than 5%, 4%, 3%, 2%, 1%, or 0.5% of their hEGFR binding activity (e.g., as measured by ELISA); and lose less than 5%, 4%, 3%, 2%, 1%, or 0.5% of their hTGFβ binding activity (e.g., as measured by ELISA).

Bifunctionality of the fusion proteins described herein can be evaluated via known methods in the art, including those described in Example 1 of the instant disclosure. For example, the bifunctionality of the fusion proteins described herein can be evaluated by two separate ELISAs or a combined ELISA that assays both functionalities of the fusion proteins described herein (i.e. specifically bind hEGFR and 2) specifically bind hTGFβ).

### Fusion Proteins

In certain aspects, provided herein are pharmaceutical compositions comprising multifunctional fusion proteins that comprises a targeting moiety and an immunomodulatory moiety, wherein: i) said targeting moiety comprises a polypeptide that specifically binds a membrane bound target protein and has a basic isoelectric point (pI); and (ii) said immunomodulatory moiety comprises a polypeptide that specifically binds a soluble target protein that has an acidic pI; wherein the membrane bound target protein and the soluble target protein are different.

In certain aspects, provided herein are pharmaceutical compositions comprising a multifunctional (e.g., bifunctional) fusion protein that comprises a targeting moiety and an immunomodulatory moiety, wherein: i) said targeting moiety specifically binds hEGFR; and (ii) said immunomodulatory moiety comprises an amino acid sequence of the extracellular domain of hTGFβRII.

hEGFR Targeting Moieties

In some embodiments, the hEGFR targeting moiety comprises an antibody, or a functional fragment or functional variant thereof. In some embodiments, the antibody is a full-length antibody, a single chain variable fragment (scFv), a scFv2, a scFv-Fc, a Fab, a Fab′, a F(ab′)2, a F(v), a single domain antibody, a single chain antibody, or a VHH.

In some embodiments, the anti-hEGFR antibody is selected from the group consisting of cetuximab and panitumumab. In some embodiments, the anti-hEGFR antibody is a functional fragment of cetuximab and panitumumab. In some embodiments, the anti-hEGFR antibody is a functional variant of cetuximab and panitumumab.

### Cetuximab

In some embodiments, the anti-hEGFR antibody is cetuximab. In some embodiments, the anti-hEGFR antibody cross-competes with cetuximab. In some embodiments, the anti-hEGFR antibody binds to the same epitope as cetuximab. In some embodiments, the anti-hEGFR antibody has the same CDRs as cetuximab.

In some embodiments the anti-hEGFR antibody comprises a variable heavy chain (VH) that comprises three complementarity determining regions: VH CDR1, VH CDR2, and VH CDR3. In some embodiments, the anti-hEGFR antibody comprises a VH comprising a VH CDR1 that comprises the amino acid sequence of SEQ ID NO: 1, with 0, 1, 2, or 3 amino acid modifications; a VH CDR2 that comprises the amino acid sequence of SEQ ID NO: 2, with 0, 1, 2, or 3 amino acid modifications; and/or a VH CDR3 that comprises the amino acid sequence of SEQ ID NO: 3, with 0, 1, 2, or 3 amino acid modifications.

In some embodiments the anti-hEGFR antibody comprises a VH comprising a VH CDR1 that comprises the amino acid sequence of SEQ ID NO: 1, or the amino acid sequence of SEQ ID NO: 1 within 1, 2, or 3 amino acid modifications; a VH CDR2 that comprises the amino acid sequence of SEQ ID NO: 2, or the amino acid sequence of SEQ ID NO: 2 within 1, 2, or 3 amino acid modifications; and/or a VH CDR3 that comprises the amino acid sequence of SEQ ID NO: 3, or the amino acid sequence of SEQ ID NO: 3 within 1, 2, or 3 amino acid modifications.

In some embodiments the anti-hEGFR antibody comprises a variable light chain (VL) that comprises three complementarity determining regions: VL CDR1, VL CDR2, and VL CDR3. In some embodiments, the anti-hEGFR antibody comprises a VL comprising a VL CDR1 that comprises the amino acid sequence of SEQ ID NO: 4, with 0, 1, 2, or 3 amino acid modifications; a VL CDR2 that comprises the amino acid sequence of SEQ ID NO: 5, with 0, 1, 2, or 3 amino acid modifications; and/or a VL CDR3 that comprises the amino acid sequence of SEQ ID NO: 6, with 0, 1, 2, or 3 amino acid modifications.

In some embodiments the anti-hEGFR antibody comprises a variable light chain (VL) that comprises three complementarity determining regions: VL CDR1, VL CDR2, and VL CDR3. In some embodiments, the anti-hEGFR antibody comprises a VL comprising a VL CDR1 that comprises the amino acid sequence of SEQ ID NO: 4, or the amino acid sequence of SEQ ID NO: 4 with 1, 2, or 3 amino acid modifications; a VL CDR2 that comprises the amino acid sequence of SEQ ID NO: 5, or the amino acid sequence of SEQ ID NO: 5 with 1, 2, or 3 amino acid modifications; and/or a VL CDR3 that comprises the amino acid sequence of SEQ ID NO: 6, or the amino acid sequence of SEQ ID NO: 6 with 1, 2, or 3 amino acid modifications.

In some embodiments, the anti-hEGFR antibody comprises a VH comprising a VH CDR1 that comprises the amino acid sequence of SEQ ID NO: 1, with 0, 1, 2, or 3 amino acid modifications; a VH CDR2 that comprises the amino acid sequence of SEQ ID NO: 2, with 0, 1, 2, or 3 amino acid modifications; and a VH CDR3 that comprises the amino acid sequence of SEQ ID NO: 3, with 0, 1, 2, or 3 amino acid modifications; and a VL comprising a VL CDR1 that comprises the amino acid sequence of SEQ ID NO: 4, with 0, 1, 2, or 3 amino acid modifications; a VL CDR2 that comprises the amino acid sequence of SEQ ID NO: 5, with 0, 1, 2, or 3 amino acid modifications; and a VL CDR3 that comprises the amino acid sequence of SEQ ID NO: 6, with 0, 1, 2, or 3 amino acid modifications.

In some embodiments, the anti-hEGFR antibody comprises a VH comprising a VH CDR1 that comprises the amino acid sequence of SEQ ID NO: 1, or the amino acid sequence of SEQ ID NO: 1 with 1, 2, or 3 amino acid modifications; a VH CDR2 that comprises the amino acid sequence of SEQ ID NO: 2, or the amino acid sequence of SEQ ID NO: 2 with 1, 2, or 3 amino acid modifications; and a VH CDR3 that comprises the amino acid sequence of SEQ ID NO: 3, or the amino acid sequence of SEQ ID NO: 3 with 1, 2, or 3 amino acid modifications; and a VL comprising a VL CDR1 that comprises the amino acid sequence of SEQ ID NO: 4, or the amino acid sequence of SEQ ID NO: 4 with 1, 2, or 3 amino acid modifications; a VL CDR2 that comprises the amino acid sequence of SEQ ID NO: 5, or the amino acid sequence of SEQ ID NO: 5 with 1, 2, or 3 amino acid modifications; and a VL CDR3 that comprises the amino acid sequence of SEQ ID NO: 6, or the amino acid sequence of SEQ ID NO: 6 with 1, 2, or 3 amino acid modifications.

In some embodiments, the anti-hEGFR antibody comprises a VH comprising a VH CDR1 that comprises an amino acid sequence at least 95%, 96%, 97%, 98%, 99%, or 100% identical to the amino acid sequence of SEQ ID NO: 1; a VH CDR2 that comprises an amino acid sequence at least 95%, 96%, 97%, 98%, 99%, or 100% to the amino acid sequence of SEQ ID NO: 2; and a VH CDR3 that comprises an amino acid sequence at least 95%, 96%, 97%, 98%, 99%, or 100% identical to the amino acid sequence of SEQ ID NO: 3.

In some embodiments, the anti-hEGFR antibody comprises a VL comprising a VL CDR1 that comprises an amino acid sequence at least 95%, 96%, 97%, 98%, 99%, or 100% identical to the amino acid sequence of SEQ ID NO: 4; a VL CDR2 that comprises an amino acid sequence at least 95%, 96%, 97%, 98%, 99%, or 100% to the amino acid sequence of SEQ ID NO: 5; and a VL CDR3 that comprises an amino acid sequence at least 95%, 96%, 97%, 98%, 99%, or 100% identical to the amino acid sequence of SEQ ID NO: 6.

In some embodiments, the anti-hEGFR antibody comprises a VH comprising a VH CDR1 that comprises an amino acid sequence at least 95%, 96%, 97%, 98%, 99%, or 100% identical to the amino acid sequence of SEQ ID NO: 1; a VH CDR2 that comprises an amino acid sequence at least 95%, 96%, 97%, 98%, 99%, or 100% of SEQ ID NO: 2; and a VH CDR3 that comprises an amino acid sequence at least 95%, 96%, 97%, 98%, 99%, or 100% identical to the amino acid sequence of SEQ ID NO: 3; and the anti-hEGFR antibody comprises a VL comprising a VL CDR1 that comprises an amino acid sequence at least 95%, 96%, 97%, 98%, 99%, or 100% identical to the amino acid sequence of SEQ ID NO: 4; a VL CDR2 that comprises an amino acid sequence at least 95%, 96%, 97%, 98%, 99%, or 100% of SEQ ID NO: 5; and a VL CDR3 that comprises an amino acid sequence at least 95%, 96%, 97%, 98%, 99%, or 100% identical to the amino acid sequence of SEQ ID NO: 6.

In some embodiments, the anti-hEGFR antibody comprises a VH at least 90%, 91%, 92%, 93%, 94%, 95%, 96%, 97%, 98%, 99%, or 100% identical to the amino acid sequence of SEQ ID NO: 7. In some embodiments, the anti-hEGFR antibody comprises a VL at least 90%, 91%, 92%, 93%, 94%, 95%, 96%, 97%, 98%, 99%, or 100% identical to the amino acid sequence of SEQ ID NO: 8. In some embodiments, the anti-hEGFR antibody comprises a VH at least 90%, 91%, 92%, 93%, 94%, 95%, 96%, 97%, 98%, 99%, or 100% identical to SEQ ID NO: 7; and a VL at least 90%, 91%, 92%, 93%, 94%, 95%, 96%, 97%, 98%, 99%, or 100% identical to the amino acid sequence of SEQ ID NO: 8.

In some embodiments, the anti-hEGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 10. In some embodiments, the anti-hEGFR antibody comprises a light chain that comprises an amino acid sequence at least 90%, 91%, 92%, 93%, 94%, 95%, 96%, 97%, 98%, 99%, or 100% identical to the amino acid sequence of SEQ ID NO: 11. In some embodiments, the anti-hEGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 10; and a light chain that comprises an amino acid sequence at least 90%, 91%, 92%, 93%, 94%, 95%, 96%, 97%, 98%, 99%, or 100% identical to the amino acid sequence of SEQ ID NO: 11.

In some embodiments, the anti-hEGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 9. In some embodiments, the anti-hEGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 9; and a light chain that comprises an amino acid sequence at least 90%, 91%, 92%, 93%, 94%, 95%, 96%, 97%, 98%, 99%, or 100% identical to the amino acid sequence of SEQ ID NO: 11.

### Panitumumab

In some embodiments, the anti-hEGFR antibody is panitumumab. In some embodiments, the anti-hEGFR antibody cross-competes with panitumumab. In some embodiments, the anti-hEGFR antibody binds to the same epitope as panitumumab. In some embodiments, the anti-hEGFR antibody has the same CDRs as panitumumab.

In some embodiments the anti-hEGFR antibody comprises a variable heavy chain (VH) that comprises three complementarity determining regions: VH CDR1, VH CDR2, and VH CDR3. In some embodiments, the anti-hEGFR antibody comprises a VH comprising a VH CDR1 that comprises the amino acid sequence of SEQ ID NO: 12, with 0, 1, 2, or 3 amino acid modifications; a VH CDR2 that comprises the amino acid sequence of SEQ ID NO: 13, with 0, 1, 2, or 3 amino acid modifications; and/or a VH CDR3 that comprises the amino acid sequence of SEQ ID NO: 14, with 0, 1, 2, or 3 amino acid modifications.

In some embodiments, the anti-hEGFR antibody comprises a VH comprising a VH CDR1 that comprises the amino acid sequence of SEQ ID NO: 12, or the amino acid sequence of SEQ ID NO: 12 with 1, 2, or 3 amino acid modifications; a VH CDR2 that comprises the amino acid sequence of SEQ ID NO: 13, or the amino acid sequence of SEQ ID NO: 13 with 1, 2, or 3 amino acid modifications; and/or a VH CDR3 that comprises the amino acid sequence of SEQ ID NO: 14, or the amino acid sequence of SEQ ID NO: 14 with 1, 2, or 3 amino acid modifications.

In some embodiments the anti-hEGFR antibody comprises a variable light chain (VL) that comprises three complementarity determining regions: VL CDR1, VL CDR2, and VL CDR3. In some embodiments, the anti-hEGFR antibody comprises a VL comprising a VL CDR1 that comprises the amino acid sequence of SEQ ID NO: 15, with 0, 1, 2, or 3 amino acid modifications; a VL CDR2 that comprises the amino acid sequence of SEQ ID NO: 16, with 0, 1, 2, or 3 amino acid modifications; and/or a VL CDR3 that comprises the amino acid sequence of SEQ ID NO: 17, with 0, 1, 2, or 3 amino acid modifications.

In some embodiments, the anti-hEGFR antibody comprises a VL comprising a VL CDR1 that comprises the amino acid sequence of SEQ ID NO: 15, or the amino acid sequence of SEQ ID NO: 15 with 1, 2, or 3 amino acid modifications; a VL CDR2 that comprises the amino acid sequence of SEQ ID NO: 16, or the amino acid sequence of SEQ ID NO: 16 with 1, 2, or 3 amino acid modifications; and/or a VL CDR3 that comprises the amino acid sequence of SEQ ID NO: 17, or the amino acid sequence of SEQ ID NO: 16 with 1, 2, or 3 amino acid modifications.

In some embodiments, the anti-hEGFR antibody comprises a VH comprising a VH CDR1 that comprises the amino acid sequence of SEQ ID NO: 12, with 0, 1, 2, or 3 amino acid modifications; a VH CDR2 that comprises the amino acid sequence of SEQ ID NO: 13, with 0, 1, 2, or 3 amino acid modifications; and a VH CDR3 that comprises the amino acid sequence of SEQ ID NO: 14, with 0, 1, 2, or 3 amino acid modifications; and a VL comprising a VL CDR1 that comprises the amino acid sequence of SEQ ID NO: 15, with 0, 1, 2, or 3 amino acid modifications; a VL CDR2 that comprises the amino acid sequence of SEQ ID NO: 16, with 0, 1, 2, or 3 amino acid modifications; and a VL CDR3 that comprises the amino acid sequence of SEQ ID NO: 17, with 0, 1, 2, or 3 amino acid modifications.

In some embodiments, the anti-hEGFR antibody comprises a VH comprising a VH CDR1 that comprises the amino acid sequence of SEQ ID NO: 12, or the amino acid sequence of SEQ ID NO: 12 with 1, 2, or 3 amino acid modifications; a VH CDR2 that comprises the amino acid sequence of SEQ ID NO: 13, or the amino acid sequence of SEQ ID NO: 13 with 1, 2, or 3 amino acid modifications; and a VH CDR3 that comprises the amino acid sequence of SEQ ID NO: 14, or the amino acid sequence of SEQ ID NO: 14 with 1, 2, or 3 amino acid modifications; and a VL comprising a VL CDR1 that comprises the amino acid sequence of SEQ ID NO: 15, or the amino acid sequence of SEQ ID NO: 15 with 1, 2, or 3 amino acid modifications; a VL CDR2 that comprises the amino acid sequence of SEQ ID NO: 16, or the amino acid sequence of SEQ ID NO: 16 with 1, 2, or 3 amino acid modifications; and a VL CDR3 that comprises the amino acid sequence of SEQ ID NO: 17, or the amino acid sequence of SEQ ID NO: 17 with 1, 2, or 3 amino acid modifications.

In some embodiments, the anti-hEGFR antibody comprises a VH comprising a VH CDR1 that comprises an amino acid sequence at least 95%, 96%, 97%, 98%, 99%, or 100% identical to the amino acid sequence of SEQ ID NO: 12; a VH CDR2 that comprises an amino acid sequence at least 95%, 96%, 97%, 98%, 99%, or 100% to the amino acid sequence of SEQ ID NO: 13; and a VH CDR3 that comprises an amino acid sequence at least 95%, 96%, 97%, 98%, 99%, or 100% identical to the amino acid sequence of SEQ ID NO: 14.

In some embodiments, the anti-hEGFR antibody comprises a VL comprising a VL CDR1 that comprises an amino acid sequence at least 95%, 96%, 97%, 98%, 99%, or 100% identical to the amino acid sequence of SEQ ID NO: 15; a VL CDR2 that comprises an amino acid sequence at least 95%, 96%, 97%, 98%, 99%, or 100% to the amino acid sequence of SEQ ID NO: 16; and a VL CDR3 that comprises an amino acid sequence at least 95%, 96%, 97%, 98%, 99%, or 100% identical to the amino acid sequence of SEQ ID NO: 17.

In some embodiments, the anti-hEGFR antibody comprises a VH comprising a VH CDR1 that comprises an amino acid sequence at least 95%, 96%, 97%, 98%, 99%, or 100% identical to the amino acid sequence of SEQ ID NO: 12; a VH CDR2 that comprises an amino acid sequence at least 95%, 96%, 97%, 98%, 99%, or 100% of SEQ ID NO: 13; and a VH CDR3 that comprises an amino acid sequence at least 95%, 96%, 97%, 98%, 99%, or 100% identical to the amino acid sequence of SEQ ID NO: 14; and the anti-hEGFR antibody comprises a VL comprising a VL CDR1 that comprises an amino acid sequence at least 95%, 96%, 97%, 98%, 99%, or 100% identical to the amino acid sequence of SEQ ID NO: 15; a VL CDR2 that comprises an amino acid sequence at least 95%, 96%, 97%, 98%, 99%, or 100% to the amino acid sequence of SEQ ID NO: 16; and a VL CDR3 that comprises an amino acid sequence at least 95%, 96%, 97%, 98%, 99%, or 100% identical to the amino acid sequence of SEQ ID NO: 17.

In some embodiments, the anti-hEGFR antibody comprises a VH at least 90%, 91%, 92%, 93%, 94%, 95%, 96%, 97%, 98%, 99%, or 100% identical to the amino acid sequence of SEQ ID NO: 18. In some embodiments, the anti-hEGFR antibody comprises a VL at least 90%, 91%, 92%, 93%, 94%, 95%, 96%, 97%, 98%, 99%, or 100% identical to the amino acid sequence of SEQ ID NO: 19. In some embodiments, the anti-hEGFR antibody comprises a VH at least 90%, 91%, 92%, 93%, 94%, 95%, 96%, 97%, 98%, 99%, or 100% identical to SEQ ID NO: 18; and a VL at least 90%, 91%, 92%, 93%, 94%, 95%, 96%, 97%, 98%, 99%, or 100% identical to the amino acid sequence of SEQ ID NO: 19.

In some embodiments, the anti-hEGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 21. In some embodiments, the anti-hEGFR antibody comprises a light chain that comprises an amino acid sequence at least 90%, 91%, 92%, 93%, 94%, 95%, 96%, 97%, 98%, 99%, or 100% identical to the amino acid sequence of SEQ ID NO: 22. In some embodiments, the anti-hEGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 21; and a light chain that comprises an amino acid sequence at least 90%, 91%, 92%, 93%, 94%, 95%, 96%, 97%, 98%, 99%, or 100% identical to the amino acid sequence of SEQ ID NO: 22.

In some embodiments, the anti-hEGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 20. In some embodiments, the anti-hEGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 20; and a light chain that comprises an amino acid sequence at least 90%, 91%, 92%, 93%, 94%, 95%, 96%, 97%, 98%, 99%, or 100% identical to the amino acid sequence of SEQ ID NO: 22.

In some embodiments, the anti-hEGFR antibody comprises an antibody in Table 1. In some embodiments, the anti-hEGFR antibody is an antibody in Table 1.

In some embodiments, the anti-hEGFR antibody comprises a VH comprising a VH CDR1 that comprises an amino acid sequence at least 95%, 96%, 97%, 98%, 99%, or 100% identical to the amino acid sequence of a VH CDR1 in Table 1; a VH CDR2 that comprises an amino acid sequence at least 95%, 96%, 97%, 98%, 99%, or 100% identical to the amino acid sequence of a VH CDR2 in Table 1; and a VH CDR3 that comprises an amino acid sequence at least 95%, 96%, 97%, 98%, 99%, or 100% identical to the amino acid sequence of a VH CDR3 in Table 1.

In some embodiments, the anti-hEGFR antibody comprises a VL comprising a VL CDR1 that comprises an amino acid sequence at least 95%, 96%, 97%, 98%, 99%, or 100% identical to the amino acid sequence of a VL CDR1 in Table 1; a VL CDR2 that comprises an amino acid sequence at least 95%, 96%, 97%, 98%, 99%, or 100% identical to the amino acid sequence of a VL CDR2 in Table 1; and a VL CDR3 that comprises an amino acid sequence at least 95%, 96%, 97%, 98%, 99%, or 100% identical to the amino acid sequence of a VL CDR3 in Table 1.

In some embodiments, the anti-hEGFR antibody comprises a VH comprising a VH CDR1 that comprises an amino acid sequence at least 95%, 96%, 97%, 98%, 99%, or 100% identical to the amino acid sequence of a VH CDR1 in Table 1; a VH CDR2 that comprises an amino acid sequence at least 95%, 96%, 97%, 98%, 99%, or 100% identical to the amino acid sequence of a VH CDR2 in Table 1; a VH CDR3 that comprises an amino acid sequence at least 95%, 96%, 97%, 98%, 99%, or 100% identical to the amino acid sequence of a VH CDR3 in Table 1; a VL comprising a VL CDR1 that comprises an amino acid sequence at least 95%, 96%, 97%, 98%, 99%, or 100% identical to the amino acid sequence of a VL CDR1 in Table 1; a VL CDR2 that comprises an amino acid sequence at least 95%, 96%, 97%, 98%, 99%, or 100% identical to the amino acid sequence of a VL CDR2 in Table 1; and a VL CDR3 that comprises an amino acid sequence at least 95%, 96%, 97%, 98%, 99%, or 100% identical to the amino acid sequence of a VL CDR3 in Table 1.

In some embodiments, the anti-hEGFR antibody comprises a heavy chain that comprises an amino acid sequence at least 90%, 91%, 92%, 93%, 94%, 95%, 96%, 97%, 98%, 99%, or 100% identical to the amino acid sequence of a heavy chain in Table 1. In some embodiments, the anti-hEGFR antibody comprises a light chain that comprises an amino acid sequence at least 90%, 91%, 92%, 93%, 94%, 95%, 96%, 97%, 98%, 99%, or 100% identical to the amino acid sequence of a light chain in Table 1. In some embodiments, the anti-hEGFR antibody comprises a heavy chain that comprises an amino acid sequence at least 90%, 91%, 92%, 93%, 94%, 95%, 96%, 97%, 98%, 99%, or 100% identical to the amino acid sequence of a heavy chain in Table 1; and a light chain that comprises an amino acid sequence at least 90%, 91%, 92%, 93%, 94%, 95%, 96%, 97%, 98%, 99%, or 100% identical to the amino acid sequence of a light chain in Table 1.

In some embodiments, the anti-hEGFR antibody comprises a VH that comprises an amino acid sequence at least 95%, 96%, 97%, 98%, 99%, or 100% identical to the amino acid sequence of a VH of an antibody in Table 1. In some embodiments, the anti-hEGFR antibody comprises a VL that comprises an amino acid sequence at least 95%, 96%, 97%, 98%, 99%, or 100% identical to the amino acid sequence of an antibody in Table 1. In some embodiments, the anti-hEGFR antibody comprises a VH that comprises an amino acid sequence at least 95%, 96%, 97%, 98%, 99%, or 100% identical to the amino acid sequence of a VH of an antibody in Table 1; and a VL that comprises an amino acid sequence at least 95%, 96%, 97%, 98%, 99%, or 100% identical to the amino acid sequence of an antibody in Table 1.

TABLE 1
Exemplary Anti-hEGFR Antibodies

| Antibody | Region | Amino Acid Sequence | SEQ ID NO |
|---|---|---|---|
| Cetuximab | VH CDR1 | NYGVH | 1 |
| (CDRs defined | VH CDR2 | VIWSGGNTDYNTPFTS | 2 |
| according to | VH CDR3 | ALTYYDYEFAY | 3 |
| Kabat) | VL CDR1 | RASQSIGTNIH | 4 |
| | VL CDR2 | YASESIS | 5 |
| | VL CDR3 | QQNNNWPTT | 6 |
| | VH | QVQLKQSGPGLVQPSQSLSITCTVSGFSLTNYGVHWVRQSPGK | 7 |
| | CDRs | GLEWLGVIWSGGNTDYNTPFTSRLSINKDNSKSQVFFKMNSLQ | |
| | Bolded | SNDTAIYYCARALTYYDYEFAYWGQGTLVTVSA | |
| | VL | DILLTQSPVILSVSPGERVSFSCRASQSIGTNIHWYQQRINGS | 8 |
| | CDRs | PRLLIKYASESISGIPSRFSGSGSGTDFTLSINSVESEDIADY | |
| | Bolded | YCQQNNNWPTTFGAGTKLELK | |
| | HC-A | QVQLKQSGPGLVQPSQSLSITCTVSGFSLTNYGVHWVRQSPGK | 9 |
| | Variable | GLEWLGVIWSGGNTDYNTPFTSRLSINKDNSKSQVFFKMNSLQ | |
| | Region | SNDTAIYYCARALTYYDYEFAYWGQGTLVTVSAASTKGPSVEP | |
| | Underlined | LAPSSKSTSGGTAALGCLVKDYFPEPVTVSWNSGALTSGVHTF | |
| | CDRs | PAVLQSSGLYSLSSVVTVPSSSLGTQTYICNVNHKPSNTKVDK | |
| | Bolded | RVEPKSCDKTHTCPPCPAPELLGGPSVFLFPPKPKDTLMISRT | |
| | | PEVTCVVVDVSHEDPEVKFNWYVDGVEVHNAKTKPREEQYNST | |
| | | YRVVSVLTVLHQDWLNGKEYKCKVSNKALPAPIEKTISKAKGQ | |
| | | PREPQVYTLPPSRDELTKNQVSLTCLVKGFYPSDIAVEWESNG | |
| | | QPENNYKTTPPVLDSDGSFFLYSKLTVDKSRWQQGNVFSCSVM | |
| | | HEALHNHYTQKSLSLSPGK | |
| | HC- | QVQLKQSGPGLVQPSQSLSITCTVSGFSLTNYGVHWVRQSPGK | 10 |
| | (No C- | GLEWLGVIWSGGNTDYNTPFTSRLSINKDNSKSQVFFKMNSLQ | |
| | terminal | SNDTAIYYCARALTYYDYEFAYWGQGTLVTVSAASTKGPSVFP | |
| | Lysine) | LAPSSKSTSGGTAALGCLVKDYFPEPVTVSWNSGALTSGVHTF | |
| | Variable | PAVLQSSGLYSLSSVVTVPSSSLGTQTYICNVNHKPSNTKVDK | |
| | Region | RVEPKSCDKTHTCPPCPAPELLGGPSVFLFPPKPKDTLMISRT | |
| | Underlined | PEVTCVVVDVSHEDPEVKFNWYVDGVEVHNAKTKPREEQYNST | |
| | CDRs | YRVVSVLTVLHQDWLNGKEYKCKVSNKALPAPIEKTISKAKGQ | |
| | Bolded | PREPQVYTLPPSRDELTKNQVSLTCLVKGFYPSDIAVEWESNG | |
| | | QPENNYKTTPPVLDSDGSFFLYSKLTVDKSRWQQGNVFSCSVM | |
| | | HEALHNHYTQKSLSLSPG | |
| | LC | DILLTQSPVILSVSPGERVSFSCRASQSIGTNIHWYQQRINGS | 11 |
| | Variable | PRLLIKYASESISGIPSRFSGSGSGTDFTLSINSVESEDIADY | |
| | Region | YCQQNNNWPTTFGAGTKLELKRTVAAPSVFIFPPSDEQLKSGT | |
| | Underlined | ASVVCLLNNFYPREAKVQWKVDNALQSGNSQESVTEQDSKDST | |
| | CDRs | YSLSSTLTLSKADYEKHKVYACEVTHQGLSSPVTKSENRGEC | |
| | Bolded | | |
| Panitumumab | VH CDR1 | SGDYYWT | 12 |
| (CDRs defined | VH CDR2 | HIYYSGNTNYNPSLKS | 13 |
| according to | VH CDR3 | DRVTGAFDI | 14 |
| Kabat) | VL CDR1 | QASQDISNYLN | 15 |
| | VL CDR2 | DASNLET | 16 |
| | VL CDR3 | QHFDHLPLA | 17 |
| | VH | QVQLQESGPGLVKPSETLSLTCTVSGGSVSSGDYYWTWIRQSP | 18 |
| | CDRs | GKGLEWIGHIYYSGNTNYNPSLKSRLTISIDTSKTQFSLKLSS | |
| | Bolded | VTAADTAIYYCVRDRVTGAFDIWGQGTMVTVSS | |
| | VL | DIQMTQSPSSLSASVGDRVTITCQASQDISNYLNWYQQKPGKA | 19 |
| | CDRs | PKLLIYDASNLETGVPSRFSGSGSGTDFTFTISSLQPEDIATY | |
| | Bolded | FCQHFDHLPLAFGGGTKVEIK | |
| | HC-A | QVQLQESGPGLVKPSETLSLTCTVSGGSVSSGDYYWTWIRQSP | 20 |
| | Variable | GKGLEWIGHIYYSGNTNYNPSLKSRLTISIDTSKTQFSLKLSS | |
| | Region | VTAADTAIYYCVRDRVTGAFDIWGQGTMVTVSSASTKGPSVFP | |
| | Underlined | LAPCSRSTSESTAALGCLVKDYFPEPVTVSWNSGALTSGVHTF | |
| | CDRs | PAVLQSSGLYSLSSVVTVPSSNFGTQTYTCNVDHKPSNTKVDK | |
| | Bolded | TVERKCCVECPPCPAPPVAGPSVFLFPPKPKDTLMISRTPEVT | |
| | | CVVVDVSHEDPEVQFNWYVDGVEVHNAKTKPREEQFNSTFRVV | |
| | | SVLTVVHQDWLNGKEYKCKVSNKGLPAPIEKTISKTKGQPREP | |
| | | QVYTLPPSREEMTKNQVSLTCLVKGFYPSDIAVEWESNGQPEN | |
| | | NYKTTPPMLDSDGSFFLYSKLTVDKSRWQQGNVFSCSVMHEAL | |
| | | HNHYTQKSLSLSPGK | |
| | HC-B | QVQLQESGPGLVKPSETLSLTCTVSGGSVSSGDYYWTWIRQSP | 21 |
| | (No C- | GKGLEWIGHIYYSGNTNYNPSLKSRLTISIDTSKTQFSLKLSS | |
| | terminal | VTAADTAIYYCVRDRVTGAFDIWGQGTMVTVSSASTKGPSVFP | |
| | Lysine) | LAPCSRSTSESTAALGCLVKDYFPEPVTVSWNSGALTSGVHTF | |
| | Variable | PAVLQSSGLYSLSSVVTVPSSNFGTQTYTCNVDHKPSNTKVDK | |
| | Region | TVERKCCVECPPCPAPPVAGPSVFLFPPKPKDTLMISRTPEVT | |
| | Underlined | CVVVDVSHEDPEVQFNWYVDGVEVHNAKTKPREEQFENSTERVV | |
| | CDRs | SVLTVVHQDWLNGKEYKCKVSNKGLPAPIEKTISKTKGQPREP | |
| | Bolded | QVYTLPPSREEMTKNQVSLTCLVKGFYPSDIAVEWESNGQPEN | |
| | | NYKTTPPMLDSDGSFFLYSKLTVDKSRWQQGNVFSCSVMHEAL | |
| | | HNHYTQKSLSLSPG | |
| | LC | DIQMTQSPSSLSASVGDRVTITCQASQDISNYLNWYQQKPGKA | 22 |
| | Variable | PKLLIYDASNLETGVPSRFSGSGSGTDFTFTISSLQPEDIATY | |
| | Region | FCQHFDHLPLAFGGGTKVEIKRTVAAPSVFIFPPSDEQLKSGT | |
| | Underlined | ASVVCLLNNFYPREAKVQWKVDNALQSGNSQESVTEQDSKDST | |
| | CDRs | YSLSSTLTLSKADYEKHKVYACEVTHQGLSSPVTKSENRGEC | |
| | Bolded | | |

hTGFβ TRAP

In certain aspects, the fusion protein comprises a targeting moiety and an immunomodulatory moiety, wherein: i) said targeting moiety specifically binds hEGFR; and (ii) said immunomodulatory moiety comprises an amino acid sequence of the extracellular domain (ECD) of hTGFβRII.

In some embodiments, the hTGFβRII ECD binds to at least one hTGFβ isoform. In some embodiments, the hTGFβRII ECD binds to hTGFβ1. In some embodiments, the hTGFβRII ECD binds to hTGFβ3. In some embodiments, the hTGFβRII ECD does not bind to hTGFβ2.

In some embodiments, the hTGFβRII ECD comprises sufficient sequence of a naturally occurring hTGFβRII ECD to enable the protein to bind hTGFβ. In some embodiments, the hTGFβRII ECD comprises sufficient sequence of a naturally occurring TGFβRII ECD to enable the protein to bind hTGFβ1. In some embodiments, the hTGFβRII ECD comprises sufficient sequence of a naturally occurring hTGFβRII ECD to enable the protein to bind hTGFβ3.

In some embodiments, the extracellular domain of hTGFβRII comprises a truncated portion of SEQ ID NO: 23, that is capable of binding hTGFβ. The extracellular domain of hTGFβRII may be truncated on the N-terminus, the C-terminus, or both the N and C terminus. The truncation may comprise the deletion of 1-10 amino acids. The truncation may comprise the deletion of 1, 2, 3, 4, 5, 6, 7, 8, 9, or 10 amino acids. The truncation may comprise the deletion of 1, 2, 3, 4, 5 amino acids from the N terminus, the C terminus, or both the N and C terminus.

In some embodiments, the extracellular domain of hTGFβRII comprises an amino acid sequence at least 90%, 91%, 92%, 93%, 94%, 95%, 96%, 97%, 98%, 99%, or 100% identical to the amino acid sequence of SEQ ID NO: 23. In some embodiments, the extracellular domain of hTGFβRII consists essentially of an amino acid sequence at least 90%, 91%, 92%, 93%, 94%, 95%, 96%, 97%, 98%, 99%, or 100% identical to the amino acid sequence of SEQ ID NO: 23. In some embodiments, the extracellular domain of hTGFβRII consists of an amino acid sequence at least 90%, 91%, 92%, 93%, 94%, 95%, 96%, 97%, 98%, 99%, or 100% identical to the amino acid sequence of SEQ ID NO: 23.

TABLE 2
Exemplary hTGFBRII ECD

| | Amino Acid Sequence | SEQ ID NO |
|---|---|---|
| hTGFβRII | TIPPHVQKSVNNDMIVTDNNGAVKFPQLCKFCDVRESTCDNQK | 23 |
| ECD | SCMSNCSITSICEKPQEVCVAVWRKNDENITLETVCHDPKLPY | |
| | HDFILEDAASPKCIMKEKKKPGETFFMCSCSSDECNDNIIFSE | |
| | EYNTSNPD | |

### Orientation

In some embodiments, the immunomodulatory moiety is operably connected to the C terminus of the targeting moiety. In some embodiments, the immunomodulatory moiety is operably connected to the N terminus of the targeting moiety.

In some embodiments, the targeting moiety is an antibody (or functional fragment or variant thereof) that comprises 1) a VH or a heavy chain, and 2) a VL or a light chain. In some embodiments, the immunomodulatory moiety is operably connected to the C terminus of the VH or heavy chain. In some embodiments, the immunomodulatory moiety is operably connected to the C terminus of the VL or light chain. In some embodiments, the immunomodulatory moiety is operably connected to the C terminus of the constant region of the heavy chain. In some embodiments, the immunomodulatory moiety is operably connected to the C terminus of the constant region of the light chain. In some embodiments, the immunomodulatory moiety is operably connected to the N terminus of the VH or heavy chain. In some embodiments, the immunomodulatory moiety is operably connected to the N terminus of the VL or light chain.

### Linkers

In some embodiments, the targeting moiety and an immunomodulatory moiety of the fusion protein are directly operably connected. In some embodiments, the targeting moiety and an immunomodulatory moiety of the fusion protein are indirectly operably connected. In some embodiments, the targeting moiety and an immunomodulatory moiety of the fusion protein are indirectly operably connected via a linker. In some embodiments, the linker is a peptide linker.

Any suitable peptide linker known in the art can be used that enables the immunomodulatory moiety and the targeting moiety to bind their respective antigens. Exemplary peptide linkers comprising glycine and serine amino acids are provided in Table 3.

In some embodiments, the linker comprises an amino acid sequence at least 95%, 96%, 97%, 98%, 99%, or 100% identical to the amino acid sequence of any one of SEQ ID NOS: 24-28. In some embodiments, the linker comprises the amino acid sequence of any one of SEQ ID NOS: 24-28, or the amino acid sequence of any one of SEQ ID NOS: 24-28 with 1, 2, or 3 amino acid modifications.

In some embodiments, the linker comprises an amino acid sequence at least 95%, 96%, 97%, 98%, 99%, or 100% identical to the amino acid sequence of SEQ ID NO: 24. In some embodiments, the linker comprises an amino acid sequence 100% identical to the amino acid sequence of SEQ ID NO: 24. In some embodiments, the linker comprises the amino acid sequence of SEQ ID NO: 24, or the amino acid sequence of SEQ ID NO: 24 with 1, 2, or 3 amino acid modifications. In some embodiments, the linker consists essentially of an amino acid sequence 100% identical to the amino acid sequence of SEQ ID NO: 24. In some embodiments, the linker consists of an amino acid sequence 100% identical to the amino acid sequence of SEQ ID NO: 24. In some embodiments, the linker consists of the amino acid sequence of SEQ ID NO: 24, or the amino acid sequence of SEQ ID NO: 24 with 1, 2, or 3 amino acid modifications.

In some embodiments, the linker comprises an amino acid sequence at least 95%, 96%, 97%, 98%, 99%, or 100% identical to the amino acid sequence of SEQ ID NO: 25. In some embodiments, the linker comprises an amino acid sequence 100% identical to the amino acid sequence of SEQ ID NO: 25. In some embodiments, the linker comprises the amino acid sequence of SEQ ID NO: 25, or the amino acid sequence of SEQ ID NO: 25 with 1, 2, or 3 amino acid modifications. In some embodiments, the linker consists essentially of an amino acid sequence 100% identical to the amino acid sequence of SEQ ID NO: 25. In some embodiments, the linker consists of an amino acid sequence 100% identical to the amino acid sequence of SEQ ID NO: 25. In some embodiments, the linker consists of the amino acid sequence of SEQ ID NO: 25, or the amino acid sequence of SEQ ID NO: 25 with 1, 2, or 3 amino acid modifications.

In some embodiments, the linker comprises an amino acid sequence at least 95%, 96%, 97%, 98%, 99%, or 100% identical to the amino acid sequence of SEQ ID NO: 26. In some embodiments, the linker comprises an amino acid sequence 100% identical to the amino acid sequence of SEQ ID NO: 26. In some embodiments, the linker comprises the amino acid sequence of SEQ ID NO: 26, or the amino acid sequence of SEQ ID NO: 26 with 1, 2, or 3 amino acid modifications. In some embodiments, the linker consists essentially of an amino acid sequence 100% identical to the amino acid sequence of SEQ ID NO: 26. In some embodiments, the linker consists of an amino acid sequence 100% identical to the amino acid sequence of SEQ ID NO: 26. In some embodiments, the linker consists of the amino acid sequence of SEQ ID NO: 26, or the amino acid sequence of SEQ ID NO: 26 with 1, 2, or 3 amino acid modifications.

In some embodiments, the linker comprises an amino acid sequence at least 95%, 96%, 97%, 98%, 99%, or 100% identical to the amino acid sequence of SEQ ID NO: 27. In some embodiments, the linker comprises an amino acid sequence 100% identical to the amino acid sequence of SEQ ID NO: 27. In some embodiments, the linker comprises the amino acid sequence of SEQ ID NO: 27, or the amino acid sequence of SEQ ID NO: 27 with 1, 2, or 3 amino acid modifications. In some embodiments, the linker consists essentially of an amino acid sequence 100% identical to the amino acid sequence of SEQ ID NO: 27. In some embodiments, the linker consists of an amino acid sequence 100% identical to the amino acid sequence of SEQ ID NO: 27. In some embodiments, the linker consists of the amino acid sequence of SEQ ID NO: 27, or the amino acid sequence of SEQ ID NO: 27 with 1, 2, or 3 amino acid modifications.

In some embodiments, the linker comprises an amino acid sequence at least 95%, 96%, 97%, 98%, 99%, or 100% identical to the amino acid sequence of SEQ ID NO: 28. In some embodiments, the linker comprises an amino acid sequence 100% identical to the amino acid sequence of SEQ ID NO: 28. In some embodiments, the linker comprises the amino acid sequence of SEQ ID NO: 28, or the amino acid sequence of SEQ ID NO: 28 with 1, 2, or 3 amino acid modifications. In some embodiments, the linker consists essentially of an amino acid sequence 100% identical to the amino acid sequence of SEQ ID NO: 28. In some embodiments, the linker consists of an amino acid sequence 100% identical to the amino acid sequence of SEQ ID NO: 28. In some embodiments, the linker consists of the amino acid sequence of SEQ ID NO: 28, or the amino acid sequence of SEQ ID NO: 28 with 1, 2, or 3 amino acid modifications.

TABLE 3
Exemplary Linkers

| Linker | Amino Acid Sequence | SEQ ID NO |
|---|---|---|
| (GGGS)₃ | GGGGSGGGGSGGGGS | 24 |
| (GGGS)₄ | GGGGSGGGGSGGGGSGGGS | 25 |
| (GGGS)₅ | GGGGSGGGGSGGGGSGGGSGGGS | 26 |
| (GGGS)₂ | GGGGSGGGGS | 27 |
| (GGGS)₁ | GGGGS | 28 |

### Exemplary Fusion Proteins

Exemplary fusion proteins of the present disclosure are provided in Table 4.

In one embodiment, the fusion protein comprises BCA101.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 10. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 29. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 10; and a light chain that comprises an amino acid sequence at least 90%, 91%, 92%, 93%, 94%, 95%, 96%, 97%, 98%, 99%, or 100% identical to the amino acid sequence of SEQ ID NO: 29.

In some embodiments, the fusion protein comprises a heavy chain that comprises an amino acid sequence 100% identical to the amino acid sequence of SEQ ID NO: 10. In some embodiments, the fusion protein comprises a light chain that comprises an amino acid sequence 100% identical to the amino acid sequence of SEQ ID NO: 29. In some embodiments, the fusion protein comprises a heavy chain that comprises an amino acid sequence 100% identical to the amino acid sequence of SEQ ID NO: 10; and a light chain that comprises an amino acid sequence 100% identical to the amino acid sequence of SEQ ID NO: 29.

In some embodiments, the fusion protein comprises a heavy chain, wherein the amino acid sequence of the heavy chain comprises the amino acid sequence of SEQ ID NO: 10. In some embodiments, the fusion protein comprises a light chain, wherein the amino acid sequence of the light chain comprises the amino acid sequence of SEQ ID NO: 29. In some embodiments, the fusion protein comprises a heavy chain, wherein the amino acid sequence of the heavy chain comprises the amino acid sequence of SEQ ID NO: 10; and a light chain, wherein the amino acid sequence of the light chain comprises the amino acid sequence of SEQ ID NO: 29.

In some embodiments, the fusion protein comprises a heavy chain that comprises the amino acid sequence of SEQ ID NO: 10, with 1, 2, or 3 amino acid modifications. In some embodiments, the fusion protein comprises a light chain that comprises the amino acid sequence of SEQ ID NO: 29, with 1, 2, or 3 amino acid modifications. In some embodiments, the fusion protein comprises a heavy chain that comprises the amino acid sequence of SEQ ID NO: 10, with 1, 2, or 3 amino acid modifications; and a light chain that comprises the amino acid sequence of SEQ ID NO: 29, with 1, 2, or 3 amino acid modifications.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 10. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 29. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 10; and a light chain that consists of an amino acid sequence at least 90%, 91%, 92%, 93%, 94%, 95%, 96%, 97%, 98%, 99%, or 100% identical to the amino acid sequence of SEQ ID NO: 29.

In some embodiments, the fusion protein comprises a heavy chain that consists of an amino acid sequence 100% identical to the amino acid sequence of SEQ ID NO: 10. In some embodiments, the fusion protein comprises a light chain that consists of an amino acid sequence 100% identical to the amino acid sequence of SEQ ID NO: 29. In some embodiments, the fusion protein comprises a heavy chain that consists of an amino acid sequence 100% identical to the amino acid sequence of SEQ ID NO: 10; and a light chain that comprises an amino acid sequence 100% identical to the amino acid sequence of SEQ ID NO: 29.

In some embodiments, the fusion protein comprises a heavy chain that consists of the amino acid sequence of SEQ ID NO: 10, with 1, 2, or 3 amino acid modifications. In some embodiments, the fusion protein comprises a light chain that consists of the amino acid sequence of SEQ ID NO: 29, with 1, 2, or 3 amino acid modifications. In some embodiments, the fusion protein comprises a heavy chain that consists of the amino acid sequence of SEQ ID NO: 10, with 1, 2, or 3 amino acid modifications; and a light chain that consists of the amino acid sequence of SEQ ID NO: 29, with 1, 2, or 3 amino acid modifications.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 30. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 11. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 30; and a light chain that comprises an amino acid sequence at least 90%, 91%, 92%, 93%, 94%, 95%, 96%, 97%, 98%, 99%, or 100% identical to the amino acid sequence of SEQ ID NO: 11.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 30. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 11. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 30; and a light chain that consists of an amino acid sequence at least 90%, 91%, 92%, 93%, 94%, 95%, 96%, 97%, 98%, 99%, or 100% identical to the amino acid sequence of SEQ ID NO: 11.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 9. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 29. In some embodiments, the fusion protein comprises a heavy chain that comprises an amino acid sequence acid sequence of SEQ ID NO: 9; and a light chain that comprises an amino acid sequence at least 90%, 91%, 92%, 93%, 94%, 95%, 96%, 97%, 98%, 99%, or 100% identical to the amino acid sequence of SEQ ID NO: 29.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 9. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 29. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 9; and a light chain that consists of an amino acid sequence at least 90%, 91%, 92%, 93%, 94%, 95%, 96%, 97%, 98%, 99%, or 100% identical to the amino acid sequence of SEQ ID NO: 29.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 31. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 11. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 31; and a light chain that comprises an amino acid sequence at least 90%, 91%, 92%, 93%, 94%, 95%, 96%, 97%, 98%, 99%, or 100% identical to the amino acid sequence of SEQ ID NO: 11.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 31. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 11. In some embodiments, the fusion protein comprises a heavy chain that consists of an amino acid sequence acid sequence of SEQ ID NO: 31; and a light chain that consists of an amino acid sequence at least 90%, 91%, 92%, 93%, 94%, 95%, 96%, 97%, 98%, 99%, or 100% identical to the amino acid sequence of SEQ ID NO: 11.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 20. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 32. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 20; and a light chain that comprises an amino acid sequence at least 90%, 91%, 92%, 93%, 94%, 95%, 96%, 97%, 98%, 99%, or 100% identical to the amino acid sequence of SEQ ID NO: 32.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 20. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 32. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 20; and a light chain that consists of an amino acid sequence at least 90%, 91%, 92%, 93%, 94%, 95%, 96%, 97%, 98%, 99%, or 100% identical to the amino acid sequence of SEQ ID NO: 32.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 33. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 22. In some embodiments, the fusion protein comprises a heavy chain that comprises an amino acid sequence acid sequence of SEQ ID NO: 33; and a light chain that comprises an amino acid sequence at least 90%, 91%, 92%, 93%, 94%, 95%, 96%, 97%, 98%, 99%, or 100% identical to the amino acid sequence of SEQ ID NO: 22.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 33. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 22. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 33; and a light chain that consists of an amino acid sequence at least 90%, 91%, 92%, 93%, 94%, 95%, 96%, 97%, 98%, 99%, or 100% identical to the amino acid sequence of SEQ ID NO: 22.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 21. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 32. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 21; and a light chain that comprises an amino acid sequence at least 90%, 91%, 92%, 93%, 94%, 95%, 96%, 97%, 98%, 99%, or 100% identical to the amino acid sequence of SEQ ID NO: 32.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 21. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 32. In some embodiments, the fusion protein comprises a heavy chain that consists of an amino acid sequence acid sequence of SEQ ID NO: 21; and a light chain that consists of an amino acid sequence at least 90%, 91%, 92%, 93%, 94%, 95%, 96%, 97%, 98%, 99%, or 100% identical to the amino acid sequence of SEQ ID NO: 32.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 34. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 22. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 34; and a light chain that comprises an amino acid sequence at least 90%, 91%, 92%, 93%, 94%, 95%, 96%, 97%, 98%, 99%, or 100% identical to the amino acid sequence of SEQ ID NO: 22.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 34. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 22. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 34; and a light chain that consists of an amino acid sequence at least 90%, 91%, 92%, 93%, 94%, 95%, 96%, 97%, 98%, 99%, or 100% identical to the amino acid sequence of SEQ ID NO: 22.

TABLE 4
Exemplary Fusion Proteins

| | | | SEQ ID |
|---|---|---|---|
| Fusion | Component | Amino Acid Sequence | NO |
| BCA101 | Heavy Chain | QVQLKQSGPGLVQPSQSLSITCTVSGFSLTNYGVHWVRQS | 10 |
| | Anti-hEGFR heavy | PGKGLEWLGVIWSGGNTDYNTPFTSRLSINKDNSKSQVFF | |
| | chain | KMNSLQSNDTAIYYCARALTYYDYEFAYWGQGTLVTVSAA | |
| | VH Underlined | STKGPSVFPLAPSSKSTSGGTAALGCLVKDYFPEPVTVSW | |
| | CDRs Underlined | NSGALTSGVHTFPAVLQSSGLYSLSSVVTVPSSSLGTQTY | |
| | & Bold | ICNVNHKPSNTKVDKRVEPKSCDKTHTCPPCPAPELLGGP | |
| | | SVFLFPPKPKDTLMISRTPEVTCVVVDVSHEDPEVKENWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPG | |
| | Light Chain | DILLTQSPVILSVSPGERVSFSCRASQSIGTNIHWYQQRT | 29 |
| | Anti-hEGFR light | NGSPRLLIKYASESISGIPSRFSGSGSGTDFTLSINSVES | |
| | chain | EDIADYYCQQNNNWPTTFGAGTKLELKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSFNRGECGGGGSGGGGSGGGGSTIPPHVQKSVN | |
| | Linker | NDMIVTDNNGAVKFPQLCKFCDVRFSTCDNQKSCMSNCSI | |
| | italicized | TSICEKPQEVCVAVWRKNDENITLETVCHDPKLPYHDFIL | |
| | hTGFβR ECD Bold | EDAASPKCIMKEKKKPGETFFMCSCSSDECNDNIIFSEEY | |
| | | NTSNPD | |
| Heavy Chain | Heavy Chain | QVQLKQSGPGLVQPSQSLSITCTVSGFSLTNYGVHWVRQS | 30 |
| Fusion | Anti-hEGFR heavy | PGKGLEWLGVIWSGGNTDYNTPFTSRLSINKDNSKSQVFF | |
| | chain | KMNSLQSNDTAIYYCARALTYYDYEFAYWGQGTLVTVSAA | |
| | VH Underlined | STKGPSVFPLAPSSKSTSGGTAALGCLVKDYFPEPVTVSW | |
| | CDRs Underlined | NSGALTSGVHTFPAVLQSSGLYSLSSVVTVPSSSLGTQTY | |
| | & Bold | ICNVNHKPSNTKVDKRVEPKSCDKTHTCPPCPAPELLGGP | |
| | Linker | SVFLFPPKPKDTLMISRTPEVTCVVVDVSHEDPEVKENWY | |
| | italicized | VDGVEVHNAKTKPREEQYNSTYRVVSVLTVLHQDWLNGKE | |
| | hTGFβR ECD Bold | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGGGGGSGGGGSGGGGSTIPPHVQKSVNNDMIVT | |
| | | DNNGAVKFPQLCKFCDVRFSTCDNQKSCMSNCSITSICEK | |
| | | PQEVCVAVWRKNDENITLETVCHDPKLPYHDFILEDAASP | |
| | | KCIMKEKKKPGETFFMCSCSSDECNDNIIFSEEYNTSNPD | |
| | Light Chain | DILLTQSPVILSVSPGERVSFSCRASQSIGTNIHWYQQRT | 11 |
| | Anti-hEGFR light | NGSPRLLIKYASESISGIPSRFSGSGSGTDFTLSINSVES | |
| | chain | EDIADYYCQQNNNWPTTFGAGTKLELKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSFNRGEC | |
| Cetuximab | Heavy Chain | QVQLKQSGPGLVQPSQSLSITCTVSGFSLTNYGVHWVRQS | 9 |
| Light Chain | Anti-hEGFR heavy | PGKGLEWLGVIWSGGNTDYNTPFTSRLSINKDNSKSQVFF | |
| Fusion | chain | KMNSLQSNDTAIYYCARALTYYDYEFAYWGQGTLVTVSAA | |
| | VH Underlined | STKGPSVFPLAPSSKSTSGGTAALGCLVKDYFPEPVTVSW | |
| | CDRs Underlined | NSGALTSGVHTFPAVLQSSGLYSLSSVVTVPSSSLGTQTY | |
| | & Bold | ICNVNHKPSNTKVDKRVEPKSCDKTHTCPPCPAPELLGGP | |
| | | SVFLFPPKPKDTLMISRTPEVTCVVVDVSHEDPEVKENWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | Light Chain | DILLTQSPVILSVSPGERVSFSCRASQSIGTNIHWYQQRT | 29 |
| | Anti-hEGFR light | NGSPRLLIKYASESISGIPSRFSGSGSGTDFTLSINSVES | |
| | chain | EDIADYYCQQNNNWPTTFGAGTKLELKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSFNRGECGGGGSGGGGSGGGGSTIPPHVQKSVN | |
| | Linker | NDMIVTDNNGAVKFPQLCKFCDVRFSTCDNQKSCMSNCSI | |
| | italicized | TSICEKPQEVCVAVWRKNDENITLETVCHDPKLPYHDFIL | |
| | hTGEβR ECD Bold | EDAASPKCIMKEKKKPGETFFMCSCSSDECNDNIIFSEEY | |
| | | NTSNPD | |
| Cetuximab | Heavy Chain | QVQLKQSGPGLVQPSQSLSITCTVSGFSLTNYGVHWVRQS | 31 |
| Heavy Chain | Anti-hEGFR heavy | PGKGLEWLGVIWSGGNTDYNTPFTSRLSINKDNSKSQVFF | |
| Fusion | chain | KMNSLQSNDTAIYYCARALTYYDYEFAYWGQGTLVTVSAA | |
| | VH Underlined | STKGPSVFPLAPSSKSTSGGTAALGCLVKDYFPEPVTVSW | |
| | CDRs Underlined | NSGALTSGVHTFPAVLQSSGLYSLSSVVTVPSSSLGTQTY | |
| | & Bold | ICNVNHKPSNTKVDKRVEPKSCDKTHTCPPCPAPELLGGP | |
| | Linker | SVFLFPPKPKDTLMISRTPEVTCVVVDVSHEDPEVKENWY | |
| | italicized | VDGVEVHNAKTKPREEQYNSTYRVVSVLTVLHQDWLNGKE | |
| | hTGFβR ECD Bold | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGKGGGGSGGGGSGGGGSTIPPHVQKSVNNDMIV | |
| | | TDNNGAVKFPQLCKFCDVRFSTCDNQKSCMSNCSITSICE | |
| | | KPQEVCVAVWRKNDENITLETVCHDPKLPYHDFILEDAAS | |
| | | PKCIMKEKKKPGETFFMCSCSSDECNDNIIFSEEYNTSNP | |
| | | D | |
| | Light Chain | DILLTQSPVILSVSPGERVSFSCRASQSIGTNIHWYQQRT | 11 |
| | Anti-hEGFR light | NGSPRLLIKYASESISGIPSRFSGSGSGTDFTLSINSVES | |
| | chain | EDIADYYCQQNNNWPTTFGAGTKLELKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSFNRGEC | |
| Panitumumab | Heavy Chain | QVQLQESGPGLVKPSETLSLTCTVSGGSVSSGDYYWTWIR | 20 |
| Light Chain | Anti-hEGFR heavy | QSPGKGLEWIGHIYYSGNTNYNPSLKSRLTISIDTSKTQF | |
| Fusion (with | chain | SLKLSSVTAADTAIYYCVRDRVTGAFDIWGQGTMVTVSSA | |
| HC C | VH Underlined | STKGPSVFPLAPCSRSTSESTAALGCLVKDYFPEPVTVSW | |
| terminal | CDRs Underlined | NSGALTSGVHTFPAVLQSSGLYSLSSVVTVPSSNFGTQTY | |
| Lysine) | & Bold | TCNVDHKPSNTKVDKTVERKCCVECPPCPAPPVAGPSVEL | |
| | | FPPKPKDTLMISRTPEVTCVVVDVSHEDPEVQFNWYVDGV | |
| | | EVHNAKTKPREEQFNSTFRVVSVLTVVHQDWLNGKEYKCK | |
| | | VSNKGLPAPIEKTISKTKGQPREPQVYTLPPSREEMTKNQ | |
| | | VSLTCLVKGFYPSDIAVEWESNGQPENNYKTTPPMLDSDG | |
| | | SFFLYSKLTVDKSRWQQGNVFSCSVMHEALHNHYTQKSLS | |
| | | LSPGK | |
| | Light Chain | DIQMTQSPSSLSASVGDRVTITCQASQDISNYLNWYQQKP | 32 |
| | Anti-hEGFR light | GKAPKLLIYDASNLETGVPSRFSGSGSGTDFTFTISSLQP | |
| | chain | EDIATYFCQHFDHLPLAFGGGTKVEIKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSFNRGECGGGGSGGGGSGGGGSTIPPHVQKSVN | |
| | Linker | NDMIVTDNNGAVKFPQLCKFCDVRESTCDNQKSCMSNCSI | |
| | italicized | TSICEKPQEVCVAVWRKNDENITLETVCHDPKLPYHDFIL | |
| | hTGFβR ECD Bold | EDAASPKCIMKEKKKPGETFFMCSCSSDECNDNIIFSEEY | |
| | | NTSNPD | |
| Panitumumab | Heavy Chain | QVQLQESGPGLVKPSETLSLTCTVSGGSVSSGDYYWTWIR | 33 |
| Heavy Chain | Anti-hEGFR heavy | QSPGKGLEWIGHIYYSGNTNYNPSLKSRLTISIDTSKTQF | |
| fusion (with | chain | SLKLSSVTAADTAIYYCVRDRVTGAFDIWGQGTMVTVSSA | |
| HC C | VH Underlined | STKGPSVFPLAPCSRSTSESTAALGCLVKDYFPEPVTVSW | |
| terminal | CDRs Underlined | NSGALTSGVHTFPAVLQSSGLYSLSSVVTVPSSNFGTQTY | |
| Lysine) | & Bold | TCNVDHKPSNTKVDKTVERKCCVECPPCPAPPVAGPSVFL | |
| | Linker | FPPKPKDTLMISRTPEVTCVVVDVSHEDPEVQFNWYVDGV | |
| | italicized | EVHNAKTKPREEQFNSTFRVVSVLTVVHQDWLNGKEYKCK | |
| | hTGFβR ECD Bold | VSNKGLPAPIEKTISKTKGQPREPQVYTLPPSREEMTKNQ | |
| | | VSLTCLVKGFYPSDIAVEWESNGQPENNYKTTPPMLDSDG | |
| | | SFFLYSKLTVDKSRWQQGNVFSCSVMHEALHNHYTQKSLS | |
| | | LSPGKGGGGSGGGGSGGGGSTIPPHVQKSVNNDMIVTDNN | |
| | | GAVKFPQLCKFCDVRFSTCDNQKSCMSNCSITSICEKPQE | |
| | | VCVAVWRKNDENITLETVCHDPKLPYHDFILEDAASPKCI | |
| | | MKEKKKPGETFFMCSCSSDECNDNIIFSEEYNTSNPD | |
| | Light Chain | DIQMTQSPSSLSASVGDRVTITCQASQDISNYLNWYQQKP | 22 |
| | Anti-hEGFR light | GKAPKLLIYDASNLETGVPSRFSGSGSGTDFTFTISSLQP | |
| | chain | EDIATYFCQHFDHLPLAFGGGTKVEIKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSENRGEC | |
| Panitumumab | Heavy Chain | QVQLQESGPGLVKPSETLSLTCTVSGGSVSSGDYYWTWIR | 21 |
| Light Chain | Anti-hEGFR heavy | QSPGKGLEWIGHIYYSGNTNYNPSLKSRLTISIDTSKTQF | |
| Fusion (with | chain | SLKLSSVTAADTAIYYCVRDRVTGAFDIWGQGTMVTVSSA | |
| HC C | VH Underlined | STKGPSVFPLAPCSRSTSESTAALGCLVKDYFPEPVTVSW | |
| terminal | CDRs Underlined | NSGALTSGVHTFPAVLQSSGLYSLSSVVTVPSSNFGTQTY | |
| Lysin) | & Bold | TCNVDHKPSNTKVDKTVERKCCVECPPCPAPPVAGPSVFL | |
| | | FPPKPKDTLMISRTPEVTCVVVDVSHEDPEVQFNWYVDGV | |
| | | EVHNAKTKPREEQFNSTFRVVSVLTVVHQDWLNGKEYKCK | |
| | | VSNKGLPAPIEKTISKTKGQPREPQVYTLPPSREEMTKNQ | |
| | | VSLTCLVKGFYPSDIAVEWESNGQPENNYKTTPPMLDSDG | |
| | | SFFLYSKLTVDKSRWQQGNVFSCSVMHEALHNHYTQKSLS | |
| | | LSPG | |
| | Light Chain | DIQMTQSPSSLSASVGDRVTITCQASQDISNYLNWYQQKP | 32 |
| | Anti-hEGFR light | GKAPKLLIYDASNLETGVPSRFSGSGSGTDFTFTISSLQP | |
| | chain | EDIATYFCQHFDHLPLAFGGGTKVEIKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSFNRGECGGGGSGGGGSGGGGSTIPPHVQKSVN | |
| | Linker | NDMIVTDNNGAVKFPQLCKFCDVRFSTCDNQKSCMSNCSI | |
| | italicized | TSICEKPQEVCVAVWRKNDENITLETVCHDPKLPYHDFIL | |
| | hTGFβR ECD Bold | EDAASPKCIMKEKKKPGETFFMCSCSSDECNDNIIFSEEY | |
| | | NTSNPD | |
| Panitumumab | Heavy Chain | QVQLQESGPGLVKPSETLSLTCTVSGGSVSSGDYYWTWIR | 34 |
| Heavy Chain | Anti-hEGFR heavy | QSPGKGLEWIGHIYYSGNTNYNPSLKSRLTISIDTSKTQF | |
| fusion (with | chain | SLKLSSVTAADTAIYYCVRDRVTGAFDIWGQGTMVTVSSA | |
| HC C | VH Underlined | STKGPSVFPLAPCSRSTSESTAALGCLVKDYFPEPVTVSW | |
| terminal | CDRs Underlined | NSGALTSGVHTFPAVLQSSGLYSLSSVVTVPSSNFGTQTY | |
| Lysine) | & Bold | TCNVDHKPSNTKVDKTVERKCCVECPPCPAPPVAGPSVEL | |
| | Linker | FPPKPKDTLMISRTPEVTCVVVDVSHEDPEVQFNWYVDGV | |
| | italicized | EVHNAKTKPREEQFNSTFRVVSVLTVVHQDWLNGKEYKCK | |
| | hTGEβR ECD Bold | VSNKGLPAPIEKTISKTKGQPREPQVYTLPPSREEMTKNQ | |
| | | VSLTCLVKGFYPSDIAVEWESNGQPENNYKTTPPMLDSDG | |
| | | SFFLYSKLTVDKSRWQQGNVFSCSVMHEALHNHYTQKSLS | |
| | | LSPGGGGGSGGGGSGGGGSTIPPHVQKSVNNDMIVTDNNG | |
| | | AVKFPQLCKFCDVRFSTCDNQKSCMSNCSITSICEKPQEV | |
| | | CVAVWRKNDENITLETVCHDPKLPYHDFILEDAASPKCIM | |
| | | KEKKKPGETFFMCSCSSDECNDNIIFSEEYNTSNPD | |
| | Light Chain | DIQMTQSPSSLSASVGDRVTITCQASQDISNYLNWYQQKP | 22 |
| | Anti-hEGFR light | GKAPKLLIYDASNLETGVPSRFSGSGSGTDFTFTISSLQP | |
| | chain | EDIATYFCQHFDHLPLAFGGGTKVEIKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSENRGEC | |

In some embodiments, the pharmaceutical composition comprises a fusion protein described herein at a concentration from about 5-50 mg/ml, 5-40 mg/ml, 5-30 mg/ml, 5-25 mg/ml, 10-50 mg/ml, 20-50 mg/ml, 25-50 mg/ml, 20-50 mg/ml, 20-40 mg/ml, 20-30 mg/ml, 25-50 mg/ml, 25-40 mg/ml, or 25-30 mg/ml. In some embodiments, the pharmaceutical composition comprises a fusion protein described herein at a concentration from about 20-30 mg/ml. In some embodiments, the pharmaceutical composition comprises a fusion protein described herein at a concentration of about 5 mg/ml, 10 mg/ml, 15 mg/ml, 20 mg/ml, 25 mg/ml, 30 mg/ml, 35 mg/ml, 40 mg/ml, 45 mg/ml, or 50 mg/ml. In some embodiments, the pharmaceutical composition comprises a fusion protein described herein at a concentration of about 25 mg/ml.

In some embodiments, the fusion protein is BCA101 at a concentration from about 50 mg/ml, 5-40 mg/ml, 5-30 mg/ml, 5-25 mg/ml, 10-50 mg/ml, 20-50 mg/ml, 25-50 mg/ml, 20-50 mg/ml, 20-40 mg/ml, 20-30 mg/ml, 25-50 mg/ml, 25-40 mg/ml, or 25-30 mg/ml. In some embodiments, the fusion protein is BCA101 and is present at a concentration from about 20-30 mg/ml. In some embodiments, the fusion protein is BCA101 and is present at a concentration of about 5 mg/ml, 10 mg/ml, 15 mg/ml, 20 mg/ml, 25 mg/ml, 30 mg/ml, 35 mg/ml, 40 mg/ml, 45 mg/ml, or 50 mg/ml. In some embodiments, the fusion protein is BCA101 and is present at a concentration of about 25 mg/ml.

In some embodiments, comprises a targeting moiety and an immunomodulatory moiety, wherein said targeting moiety comprises an antibody that comprises a heavy chain comprising an amino acid sequence at least 95%, 96%, 97%, 98%, 99%, or 100% identical to the amino acid sequence of SEQ ID NO: 10; and a light chain comprising an amino acid sequence at least 95%, 96%, 97%, 98%, 99%, or 100% identical to the amino acid sequence of SEQ ID NO: 29, and is present in the pharmaceutical composition at a concentration from about 20-30 mg/ml. In some embodiments, comprises a targeting moiety and an immunomodulatory moiety, wherein said targeting moiety comprises an antibody that comprises a heavy chain comprising an amino acid sequence at least 95%, 96%, 97%, 98%, 99%, or 100% identical to the amino acid sequence of SEQ ID NO: 10; and a light chain comprising an amino acid sequence at least 95%, 96%, 97%, 98%, 99%, or 100% identical to the amino acid sequence of SEQ ID NO: 29, and is present in the pharmaceutical composition at a concentration from about 25 mg/ml.

### Methods of Manufacture

In some aspects, provided herein are methods of manufacturing pharmaceutical compositions described herein. In some embodiments, the method comprises culturing mammalian cells comprising one or more nucleic acids encoding a fusion protein described herein in a cell culture medium such that the cells secrete said fusion protein into the cell culture medium; purifying the fusion protein from the cell culture media; and preparing a pharmaceutical composition described herein.

In some embodiments, the cells have stably incorporated the one or more nucleic acids encoding a fusion protein into their genome. In some embodiments, the cells have transiently incorporated one or more nucleic acids encoding a fusion protein into the cell. In some embodiments, the one or more nucleic acids encoding a fusion protein is introduced into the cell via transfection or transduction. Transfection and transduction methods are well known in the art.

Any suitable mammalian cell can be used for expression of the fusion protein. For example, well known mammalian cells include, but are not limited to, monkey kidney CV1 line transformed by SV40 (COS-7, ATCC CRL 1651), human embryonic kidney line (293 or 293 cells subcloned for growth in suspension culture, (Graham et al., 1977, J. Gen Virol. 36: 59), baby hamster kidney cells (BHK, ATCC CCL 10), Chinese hamster ovary cells/-DHFR1 (CHO, Urlaub et al., 1980, Proc. Natl. Acad. Sci. USA 77: 4216; e.g., DG44), mouse sertoli cells (TM4, Mather, 1980, Biol. Reprod. 23:243-251), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TRI cells (Mather et al, 1982, Annals N.Y. Acad. Sci. 383: 44-68), MRC 5 cells, FS4 cells, and human hepatoma line (Hep G2).

The host cells used to produce a fusion protein described herein may be cultured in a variety of media. Commercially available media such as Ham's F10 (Sigma-Aldrich Co, St. Louis, Mo.), Minimal Essential Medium ((MEM), (Sigma-Aldrich Co.), RPML 1640 (Sigma-Aldrich Co.), and Dulbecco's Modified Eagle's Medium ((DMEM), Sigma-Aldrich Co.) are suitable for culturing the host cells. In addition, any of the media described in one or more of Ham et al, 1979, Meth. Enz. 58: 44, Barnes et al, 1980, Anal. Biochem. 102: 255, U.S. Pat. Nos. 4,767,704, 4,657,866, 4,927,762, 4,560,655, 5,122, 469, WO 90/103430, and WO 87/00195 may be used as culture media for the host cells, the full contents of which are incorporated by reference herein.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamicin), trace elements (defined as inorganic compounds usually present at final concentrations in the micromolar range), and glucose or an equivalent energy source. Other supplements may also be included at appropriate concentrations that would be known to those skilled in the art. The culture conditions, such as temperature, pH, and the like, are those previously used with the host cell selected for expression, and will be apparent to the ordinarily skilled artisan.

The fusion protein secreted from the cells can be purified using any suitable method known in the art. For example, size exclusion chromatography, hydroxylapatite chromatography, affinity chromatography, gel electrophoresis, dialysis, or tangential flow filtration. In some embodiments, the fusion protein or the pharmaceutical composition undergoes sterile filtration. In some embodiments, the pharmaceutical composition is produced as a drug substance and undergoes sterile filtration to produce drug product.

### Therapeutic Uses

In one aspect, provided herein are methods of treating cancer in a subject by administering to the subject having cancer a pharmaceutical composition described herein.

In some embodiments, the methods disclosed herein are used in place of standard of care therapies. In certain embodiments, a standard of care therapy is used in combination with any method disclosed herein. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers. In some embodiments, the methods disclosed herein are used after standard of care therapy has failed.

### Exemplary Cancers

In some embodiments, the cancer is metastatic. In some embodiments, the cancer is recurrent. In some embodiments, the cancer is metastatic and recurrent.

In some embodiments, the cancer is EGFR-driven. In some embodiments, the cancer is a solid tumor. In some embodiments, the cancer is a hematological malignancy.

In some embodiments, said cancer is metastatic. In some embodiments, said cancer is recurrent. In some embodiments, said cancer is refractory. In some embodiments, said cancer is metastatic, recurrent, and/or refractory, or any combination thereof.

In some embodiments, said cancer comprises cancer cells that contain a genomic amplification of the EGFR gene, e.g., as detected by biopsy and fluorescence in situ hybridization.

In some embodiments, said cancer comprises cancer cells that contain a genomic modification in the KRAS gene. In some embodiments, said modification in the KRAS gene is a G12D substitution. In some embodiments, said modification in the KRAS gene is a G13D modification.

In some embodiments, said cancer is selected from the group consisting of eye, stomach, colon, rectum, colorectal, breast cancer, anal cancer, pancreatic cancer, thyroid cancer, liver cancer, ovarian cancer, lung cancer, skin cancer, brain cancer, spinal cord cancer, head cancer, and neck cancer.

In some embodiments, said cancer is lung cancer. In some embodiments, said cancer is squamous cell lung cancer (SqCLC). In some embodiments, said SqCLC comprises cancer cells that does not express detectable levels of programmed death-ligand 1, as measured by a biopsy. In some embodiments, said SqCLC comprises cancer cells that contain a genomic amplification of the EGFR gene, e.g., as detected by biopsy and fluorescence in situ hybridization.

In some embodiments, said cancer is colorectal cancer. In some embodiments, said colorectal cancer is RAS wild-type microsatellite stable Colorectal Carcinoma (RAS WT MSS CRC). In some embodiments, said cancer is breast cancer. In some embodiments, said cancer is triple negative breast cancer (TNBC).

In some embodiments, said cancer is a spinal cord cancer. In some embodiments, said cancer of the spinal cord is a chordoma. In some embodiments, said cancer is a cancer of the eye. In some embodiments, said cancer of the eye is a melanoma of the eye. In some embodiments, said cancer is a brain cancer. In some embodiments, said brain cancer is a glioblastoma.

In some embodiments, said cancer is ovarian cancer. In some embodiments, said ovarian cancer is epithelial ovarian cancer. In some embodiments, said cancer is liver cancer. In some embodiments, said liver cancer is hepatocellular carcinoma (HCC). In some embodiments, said cancer is thyroid cancer. In some embodiments, said thyroid cancer is anaplastic thyroid cancer (ATC). In some embodiments, said cancer is pancreatic cancer. In some embodiments, said cancer is stomach cancer.

In some embodiments, the cancer is head and neck cancer. In some embodiments, the cancer is head and neck squamous cell carcinoma (HNSCC). In some embodiments, the cancer is recurrent HNSCC. In some embodiments, the cancer is metastatic HNSCC. In some embodiments, the cancer is metastatic and recurrent HNSCC. In some embodiments, the cancer is anal canal. In some embodiments, the cancer is squamous cell carcinoma of anal canal (SCCAC). In some embodiments, the cancer is recurrent SCCAC. In some embodiments, the cancer is metastatic SCCAC. In some embodiments, the cancer is metastatic and recurrent SCCAC.

### Exemplary Dosing Regimens and Schedules

In some embodiments, the fusion protein (i.e. fusion protein that comprises a targeting moiety and an immunomodulatory moiety, wherein: i) said targeting moiety specifically binds hEGFR; and (ii) said immunomodulatory moiety comprises an amino acid sequence of the extracellular domain of hTGFβRII), is administered to the subject having cancer at a therapeutically effective dose. In some embodiments, the fusion protein is administered to the subject having cancer at a fixed dose. In some embodiments, the fusion protein is administered to the subject having cancer at a flat dose. In some embodiments, the fusion protein is administered to the subject having cancer at a weight based dose.

In some embodiments, the fusion protein is administered to the subject at a dose from about 50 mg to 2000 mg, 100 mg to 2000 mg, 150 mg to 2000 mg, 200 mg to 2000 mg, 300 mg to 2000 mg, 400 mg to 2000 mg, 500 mg to 2000 mg, 600 mg to 2000 mg, 700 mg to 2000 mg, 800 mg to 2000 mg, 9000 mg to 2000 mg, 1000 mg to 2000 mg, 1500 mg to 2000 mg, 50 mg to 100 mg, 50 mg to 500 mg, 50 mg to 400 mg, 50 mg to 300 mg, 50 mg to 200 mg, 50 mg to 100 mg, 100 mg to 500 mg, 100 mg to 400 mg, 100 mg to 300 mg, or 100 mg to 200 mg. In some embodiments, the fusion protein is administered to the subject at a dose of from about 200 mg to 2000 mg. In some embodiments, the fusion protein is administered to the subject at a dose of about 50 mg, 60 mg, 64 mg, 100 mg, 150 mg, 200 mg, 240 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900, or 2000 mg. In some embodiments, the fusion protein is administered to the subject at a dose of about 64 mg, 240 mg, 800 mg, or 1600 mg. In some embodiments, the fusion protein is administered to the subject at a dose of about 64 mg. In some embodiments, the fusion protein is administered to the subject at a dose of about 240 mg. In some embodiments, the fusion protein is administered to the subject at a dose of about 800 mg. In some embodiments, the fusion protein is administered to the subject at a dose of about 1600 mg.

In some embodiments, the fusion protein is administered to the subject every 1, 2, 3, 4, 5, or 6 weeks. In some embodiments, the fusion protein is administered to the subject every week. In some embodiments, the fusion protein is administered to the subject every 2 weeks. In some embodiments, the fusion protein is administered to the subject every 3 weeks. In some embodiments, the fusion protein is administered to the subject every 4 weeks. In some embodiments, the fusion protein is administered to the subject 5 weeks. In some embodiments, the fusion protein is administered to the subject every 6 weeks.

### Kits

In one aspect, provided herein are kits comprising a liquid pharmaceutical composition described herein for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a cancer, the kit comprising: (a) a dosage of pharmaceutical composition described herein and (b) instructions for using the in methods of therapy methods disclosed herein. In certain embodiments for treating human patients, the kit comprises a liquid pharmaceutical composition described herein comprising BCA101.

The present invention is further illustrated by the following examples which should not be construed as further limiting. The contents of all references cited throughout this application are expressly incorporated herein by reference.

### EXAMPLES

### Example 1. Development of BCA101 Formulation

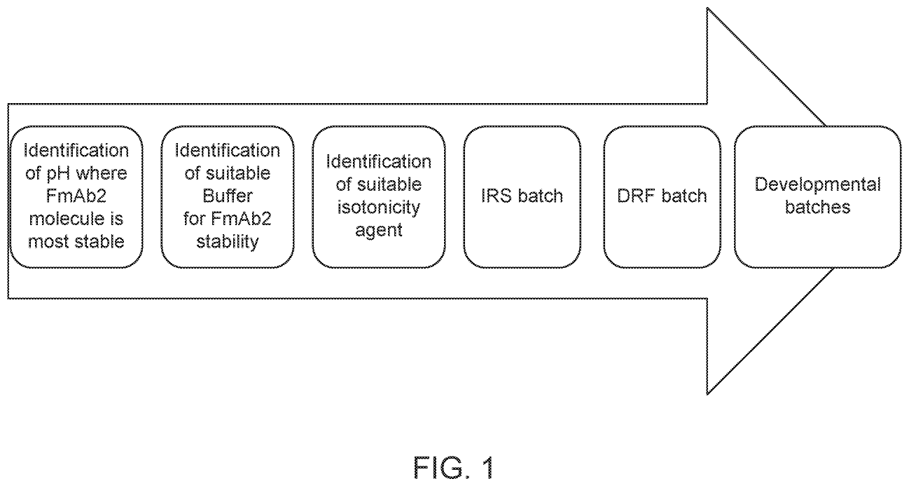

The objective of this study was to develop formulations for bifunctional fusion proteins wherein the two functional domains of the protein have different isoelectric points. This study utilizes BCA101 as such a fusion protein. BCA101, as described herein, is a bifunctional fusion protein that comprises an anti-hEGFR antibody and the extracellular domain of hTGFβRII fused to the C-terminus of the anti-hEGFR antibody light chains. The anti-hEGFR antibody domain of BCA101 has a basic pI (isoelectric point), while the hTGFβRII extracellular domain has an acidic pI. Thus, a formulation needed to be developed that could accommodate a fusion protein comprising two proteins of different function, structure, and pI. The formulation would need to maintain the physiochemical stability of the fusion protein, functional and biological potency of the fusion upon long term storage (e.g., 12-24 months) at refrigerated (e.g., 2-8° C.) or frozen (e.g., −20° C.) temperatures. The formulation was developed through a series of studies as shown in FIG. 1.

### pH Screening Study

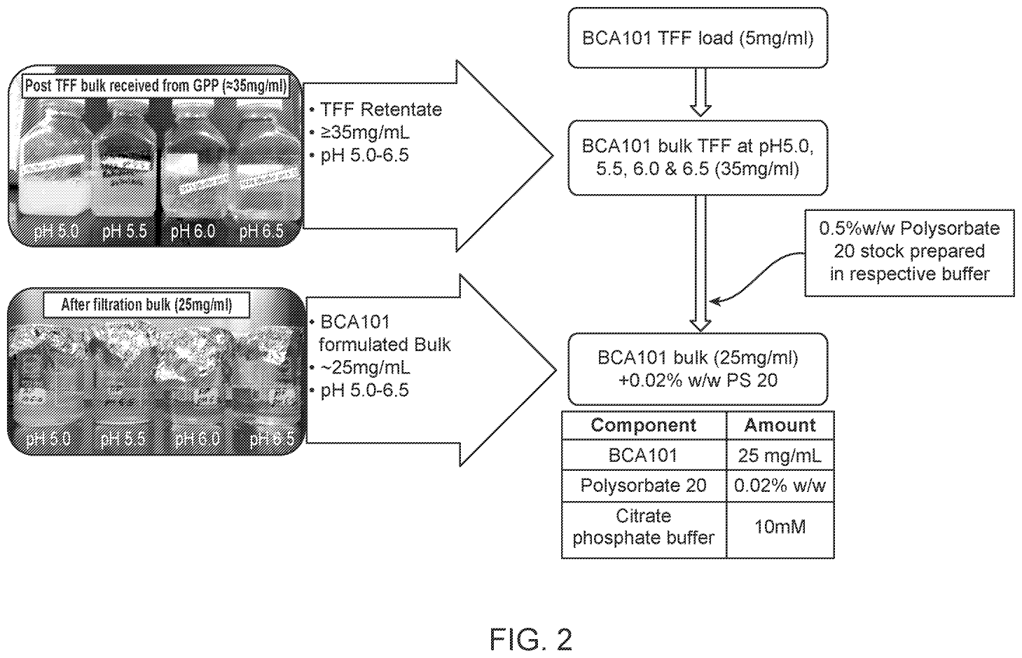

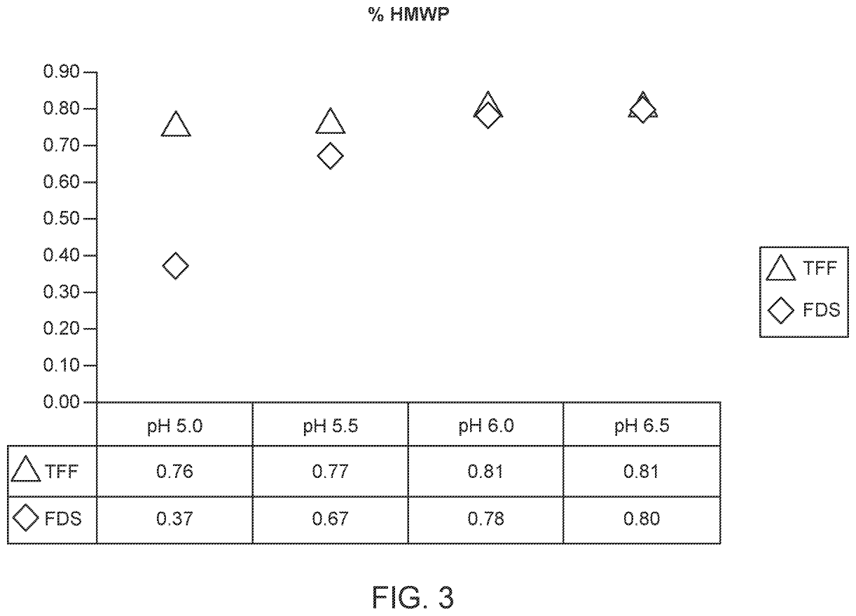

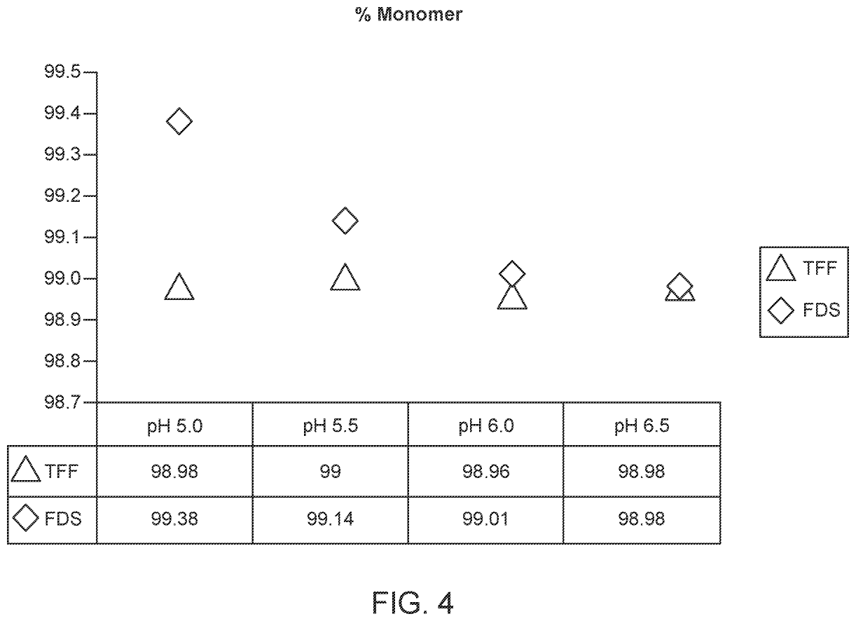

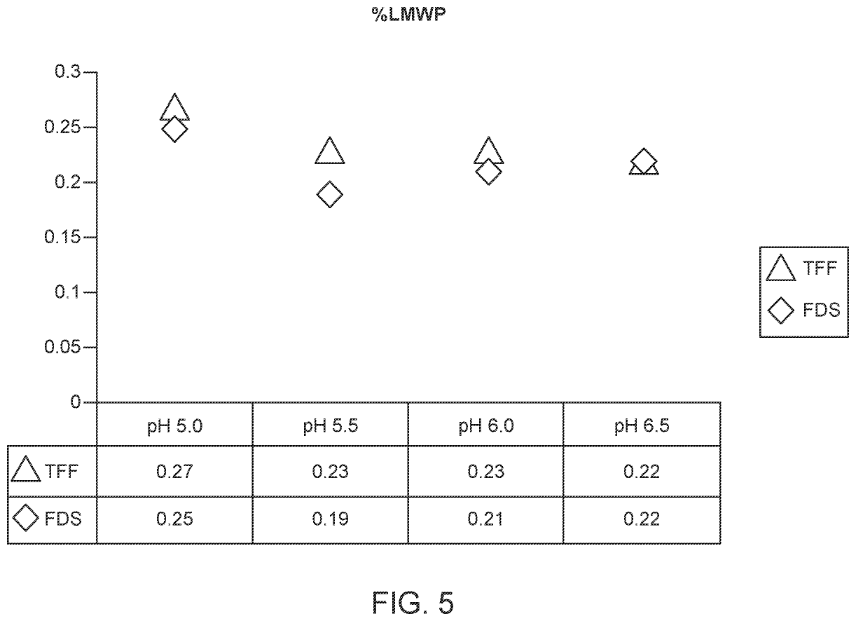

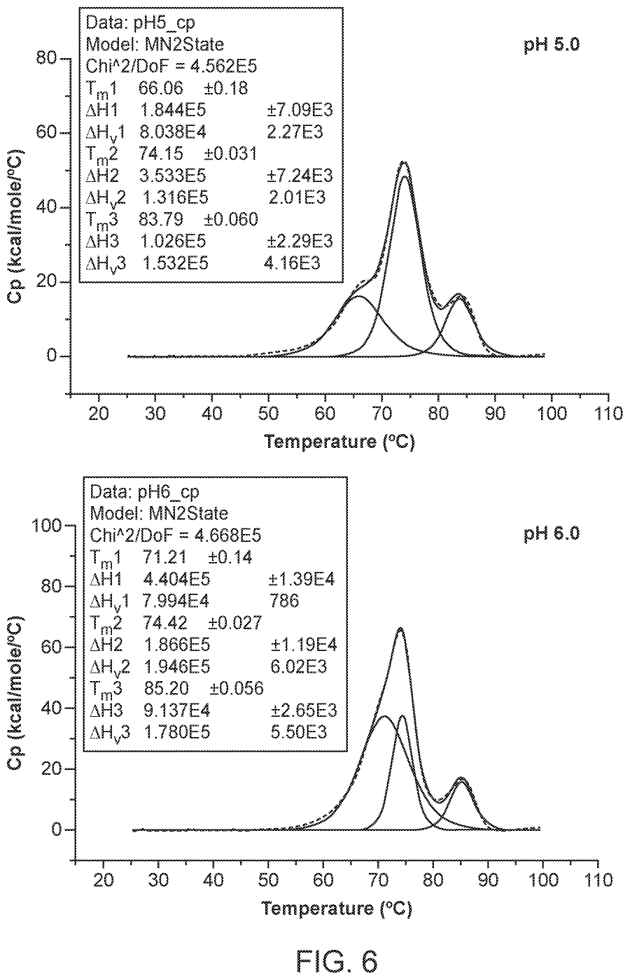

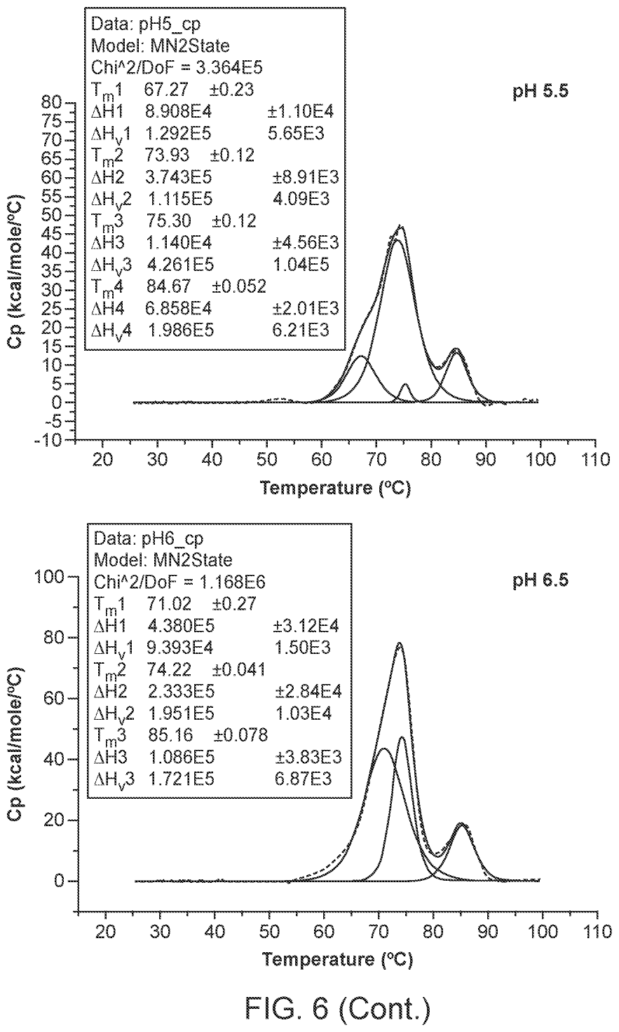

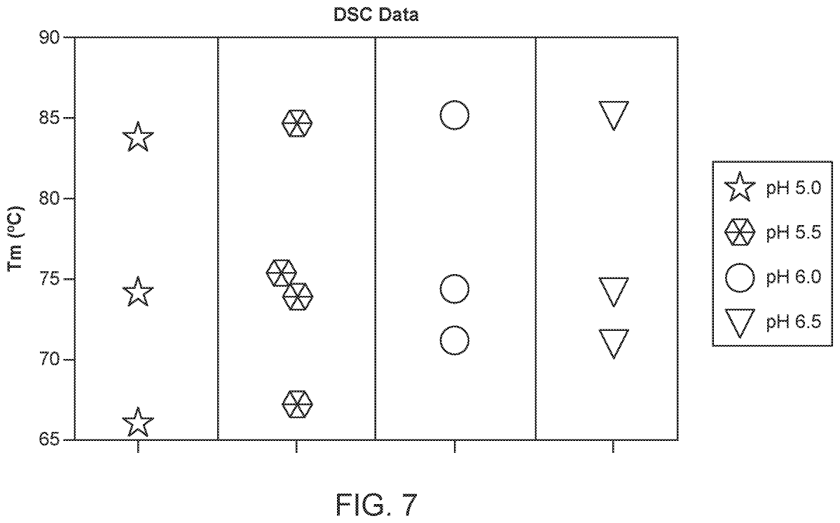

A pH screening study was conducted in order to determine the pH where BCA101 is the most stable. BCA101 (˜35 mg/ml) ultrafiltration was carried out using tangential flow filtration (TFF) at a pH of 5.0, 5.5, 6.0, and 6.5, the filtration product was formulated in 10 mM citrate phosphate buffer (10 mM), 0.02% w/v polysorbate 20, and 25 mg/ml BCA101 (FIG. 2). The percent of high molecular weight protein (HMWP) (FIG. 3), percent protein monomer (FIG. 4), and percent low molecular weight protein (LMWP) (FIG. 5) were measured via size exclusion chromatography for the bulk tangential flow filtration composition (TFF) and the final drug product (FDP). The stability of BCA101 in each of the pH formulations was further analyzed by differential scanning calorimetry (DSC). The DSC line graphs for each formulation are shown in FIG. 6, with a summary of the data at each pH shown in FIG. 7 and Table 5.

TABLE 5
Summary DSC Data for each pH

Formulation pH

| Tm | pH 5.0 | pH 5.5 | pH 6.0 | pH 6.5 |
|---|---|---|---|---|
| Tm 1 | 66.06 | 67.27 | 71.21 | 71.02 |
| Tm 2 | 74.15 | 73.93 | 74.42 | 74.22 |
| Tm 3 | 83.79 | 75.30 | 85.20 | 85.16 |
| Tm 4 | | 84.67 | | |

*Age at the time of sample analysis: 12 months stored at 2-8° C.; Scan Rate: 90° C./hour

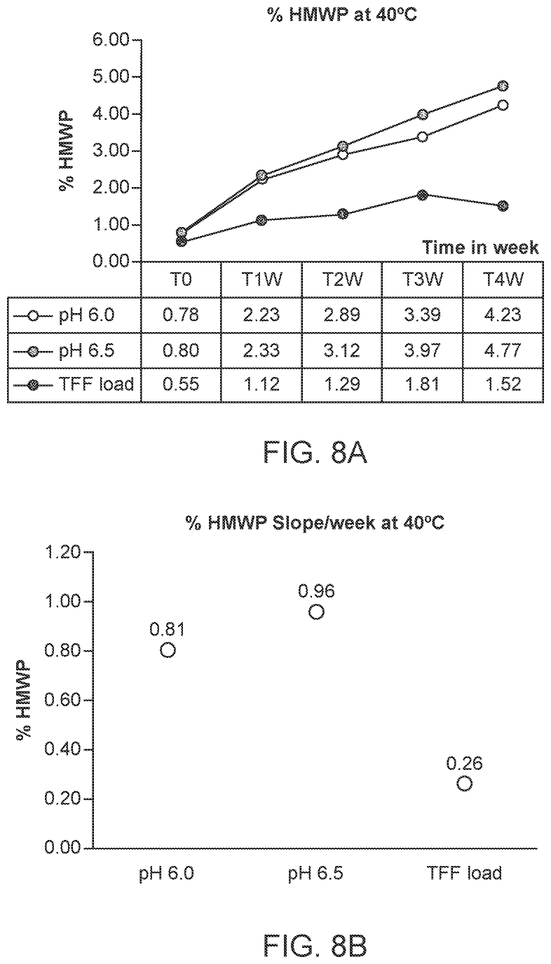

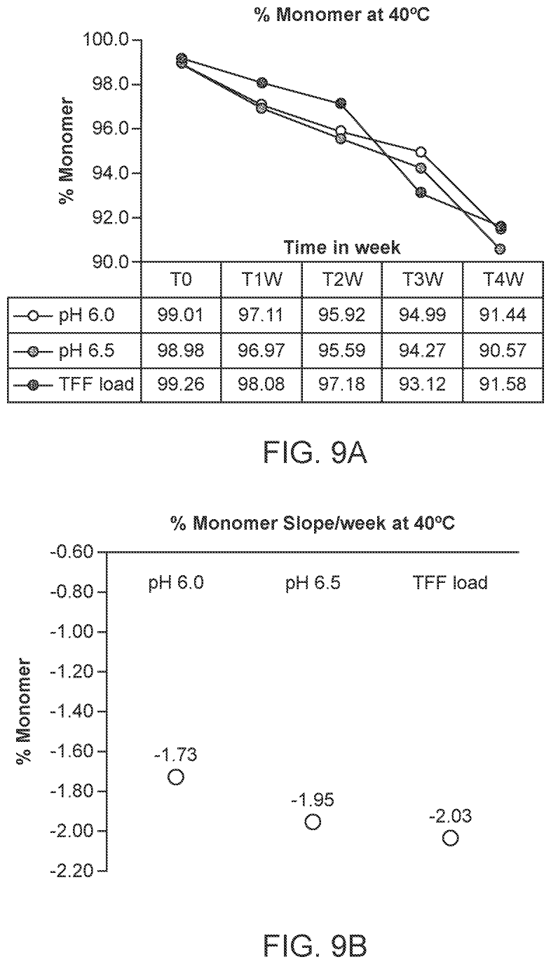

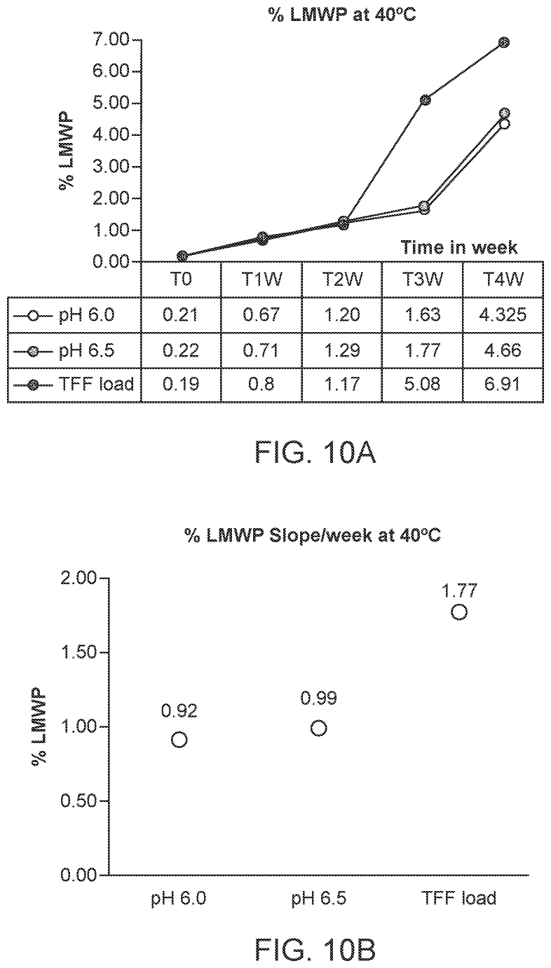

The percent of HMWP (FIGS. 8A-8B), percent monomer (FIGS. 9A-9B), and percent LMWP (FIGS. 10A-10B) at 40° C. were further determined for the formulations at pH 6.0 and pH 6.5. Based on the above data, pH 6.0 and 6.5 formulations were selected for further development.

### Buffer Screening Study

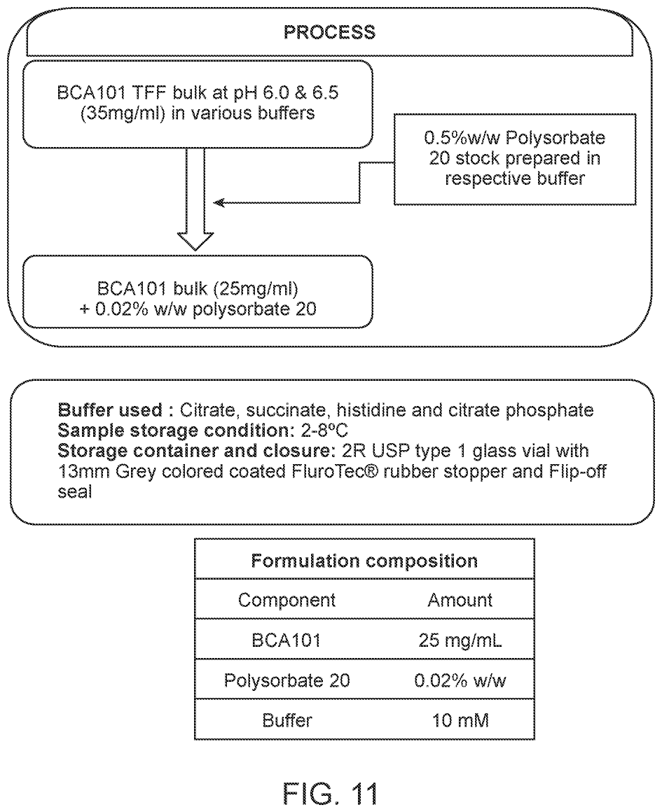

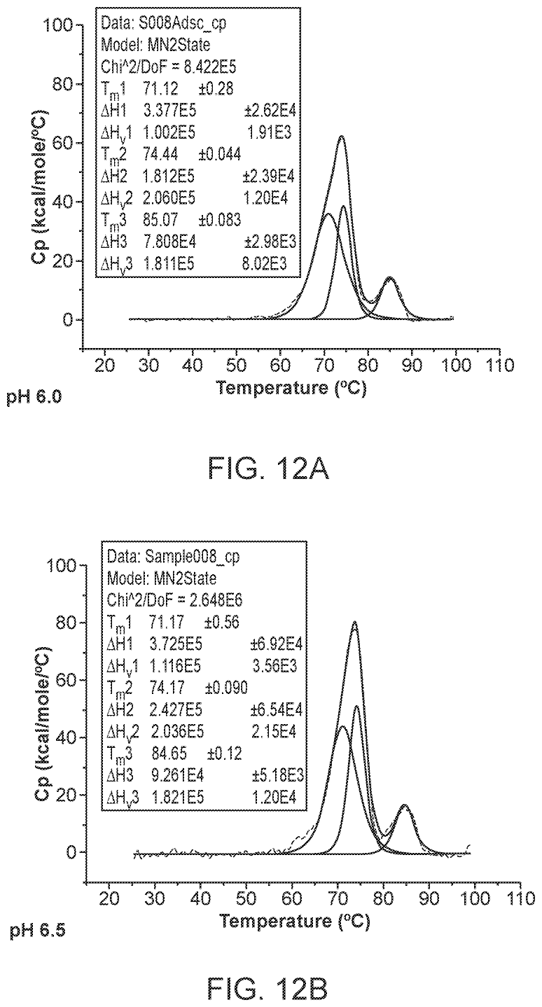

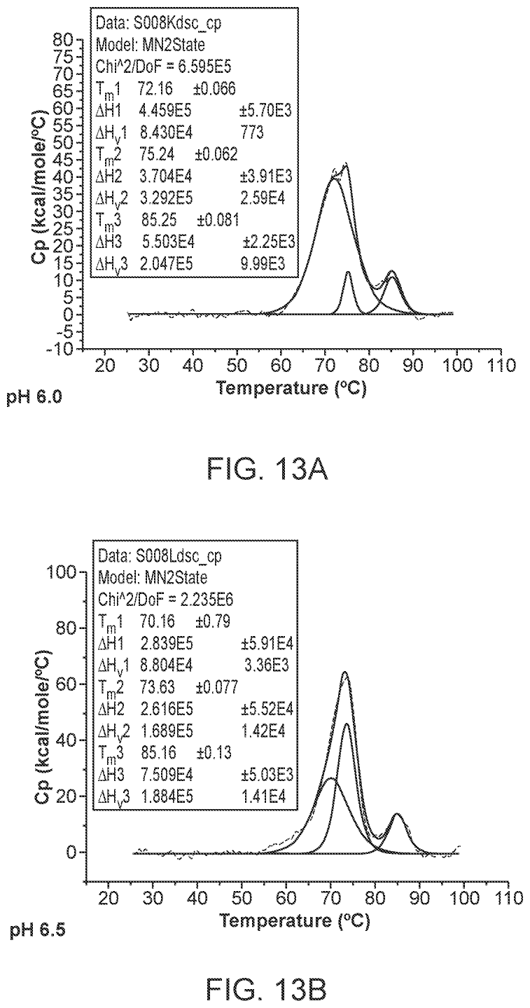

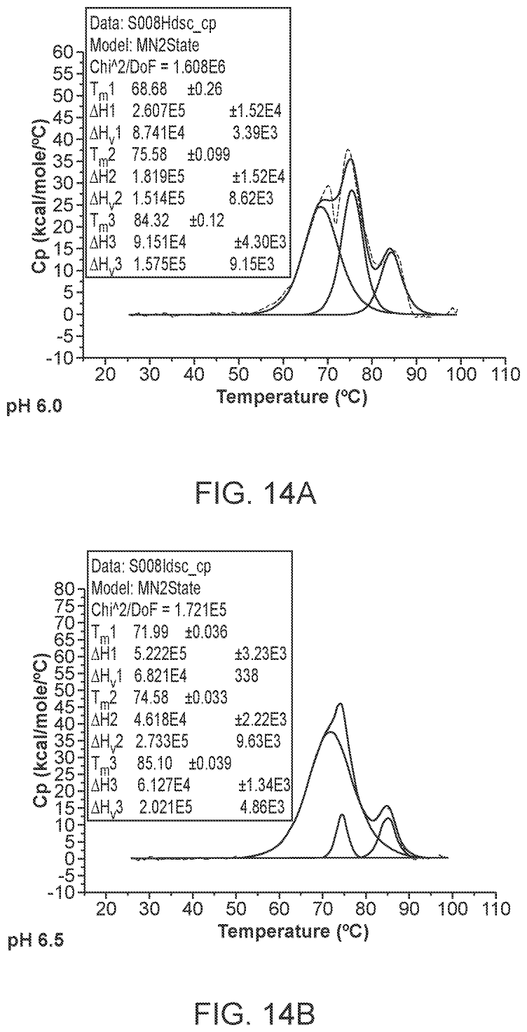

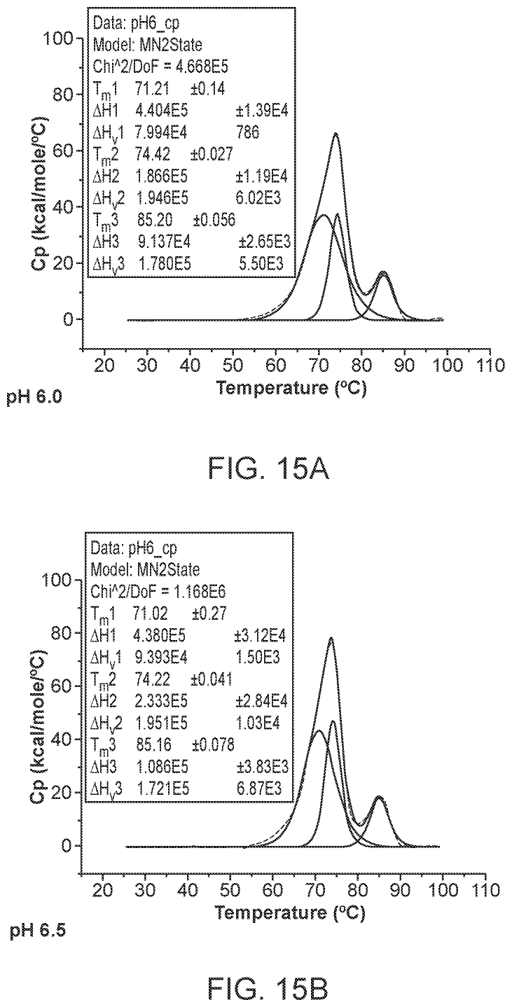

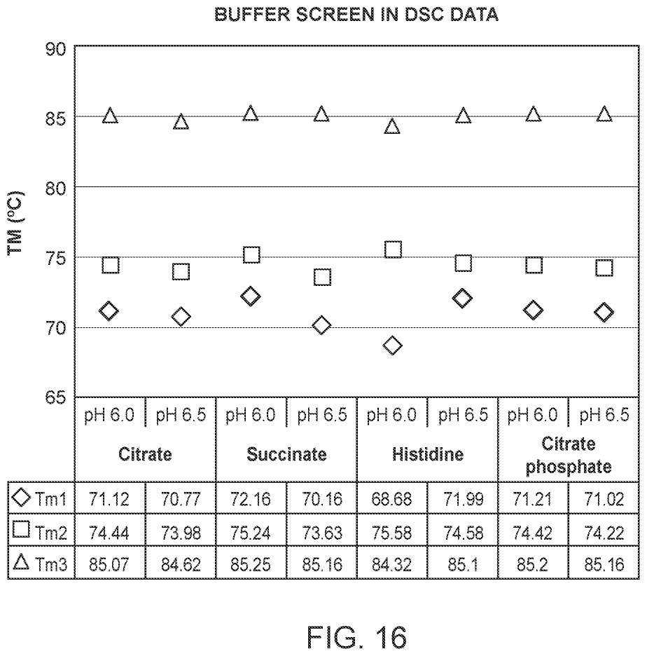

Four different buffers (citrate, succinate, histidine, and citrate phosphate (citric acid monohydrate (0.573 mg/mL), Disodium Hydrogen phosphate dihydrate (1.294 mg/mL))) were tested at the selected pHs of 6.0 and 6.5. The drug products were stored at 2-8° C. in 2R USP type 1 glass vials with 13 mm grey color coated FluroTec® rubber stopper and flip off seal. Each formulation contained 25 mg/ml BCA101, 0.02% w/v polysorbate 20, and 10 mm of the test buffer (citrate, succinate, histidine, and citrate phosphate) (FIG. 11). The stability of BCA101 was measured for each formulation by DSC: citrate buffer (FIGS. 12A-12B), succinate buffer (FIGS. 13A-13B), histidine buffer (FIGS. 14A-14B), and citrate phosphate buffer (FIGS. 15A-15B). A summary of the DSC data across the four test buffers is presented in FIG. 16.

Each formulation was further evaluated for physical appearance, filterability, Tm (by DSC), and HMWP (stress stability) (Table 6). Based on the above data, citrate phosphate (pH 6.0) and succinate (pH 6.5) were selected for further development.

TABLE 6
DSC data for each test buffer at pH 6.0 and 6.5

| | Formulation | | | | | | | |
|---|---|---|---|---|---|---|---|---|
| | | | | | | | Citrate | |
| | Citrate | | Succinate | | Histidine | | Phosphate | |
| | Buffer | | Buffer | | Buffer | | Buffer | |
| Parameter | pH | | pH | | pH | | pH | |
| Evaluated | 6.0 | 6.5 | 6.0 | 6.5 | 6.0 | 6.5 | 6.0 | 6.5 |
| Physical | Clear | Clear | Clear | Clear | Cloudy | Clear | Clear | Clear |
| Appearance | | | | | | | | |
| Filterability | 4 | 4 | 4 | 4 | 1 | 4 | 4 | 4 |
| Tm by DSC | 4 | 3 | 4 | 4 | 3 | 4 | 4 | 4 |
| HMWP | 1 | 4 | 1 | 4 | 1 | 1 | 4 | 3 |
| (stress | | | | | | | | |
| stability) | | | | | | | | |

### Tonicity Modifier Screening

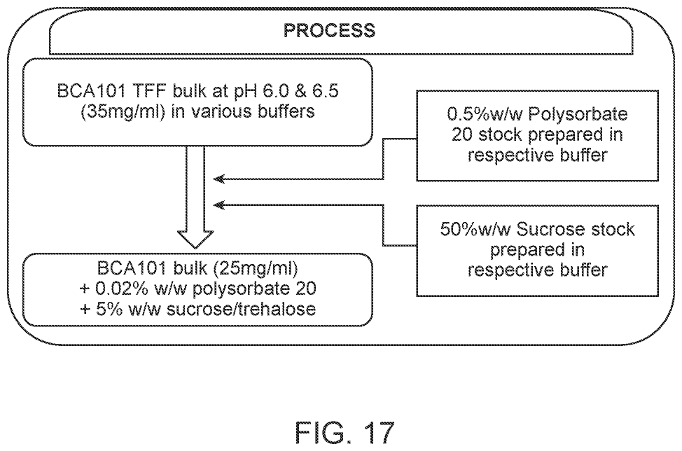

Tonicity modifiers were screened for each buffer and pH selected above. Each test formulation contained 25 mg/ml BCA101, 0.02% w/w, 5.0% w/v tonicity modifier (sucrose or trehalose), and 10 mM buffer (citrate phosphate (pH 6.0) or succinate (pH 6.5)) (FIG. 17), according to Table 7 below.

TABLE 7
Formulations for Tonicity Modifier Screen

| | BCA101 drug product (BR.14.09015/R/16/0019) | | | | |
|---|---|---|---|---|---|
| | | | | | Polysorbate |
| | | | Sucrose | Trehalose | 20 |
| | Batch | | (5% | (5% | (0.02% |
| Batch No. | No. | Buffer | w/v) | w/v) | w/v) |
| BL.14.0901/ | A | Citrate | ✓ | X | ✓ |
| 16/006 | B | Phosphate | X | ✓ | ✓ |
| | C | pH 6.0 | X | X | ✓ |
| | (Control) | | | | |
| BL.14.0901/ | D | Succinate | ✓ | X | ✓ |
| 16/007 | E | pH 6.5 | X | ✓ | ✓ |
| | F | | X | X | ✓ |
| | (Control) | | | | |

### Stress Stability Study

A stress stability study was carried out at 40° C., and a freeze thaw study wherein 3 freeze that cycles were carried out in cryovials, wherein each freeze cycle was 48 hours freezing at −80° C. and −20° C., and each thawing cycle was 4 hours of thawing at 25° C. in an incubator. The results of the freeze thaw and stress stability studies are summarized in Table 8. There was no change observed in pH, osmolality, or protein concentration in either the freeze/thaw study or stress study for any of the test formulations.

TABLE 8
Stability and freeze/thaw study results

| | Citrate Phosphate Buffer (pH 6.0) | | | Succinate Buffer (pH 6.5) | | |
|---|---|---|---|---|---|---|
| Study | Control | Sucrose | Trehalose | Control | Sucrose | Trehalose |
| Freeze/Thaw (FT) | 4 | 3 | 3 | | 1 | 1 |
| Stress Stability | 1 | 4 | 2 | 4 | Not | 3 |
| | | | | | Available | |
| | | | | | (NAV) | |

The osmolality of the sucrose formulation was evaluated (Table 9). 5% w/v resulted in osmolality values in the range of 170-240 mOsmol/kg. The sucrose concentration was further optimized based on achieving a target osmolality value of 300 mOsmol/kg. Osmolality of BCA101 formulation with 0.02% w/v polysorbate-20 and 10 mM citrate phosphate buffer, in absence of sucrose was in the range of 30-35 mOsmol/Kg. 8.0% w/v sucrose concentration is considered to achieve 300 mOsmol/kg for BCA101 drug product.

TABLE 9
Sucrose concentration optimization and osmolality

| Sucrose | |
|---|---|
| Concentration | Osmolality |
| (% w/v) | (mOsmol/Kg) |
| 7.0 | 231 |
| 7.5 | 253 |
| 8.0 | 270 |
| 8.5 | 287 |
| 9.0 | 306 |
| 9.5 | 324 |

### Color and Clarity Study

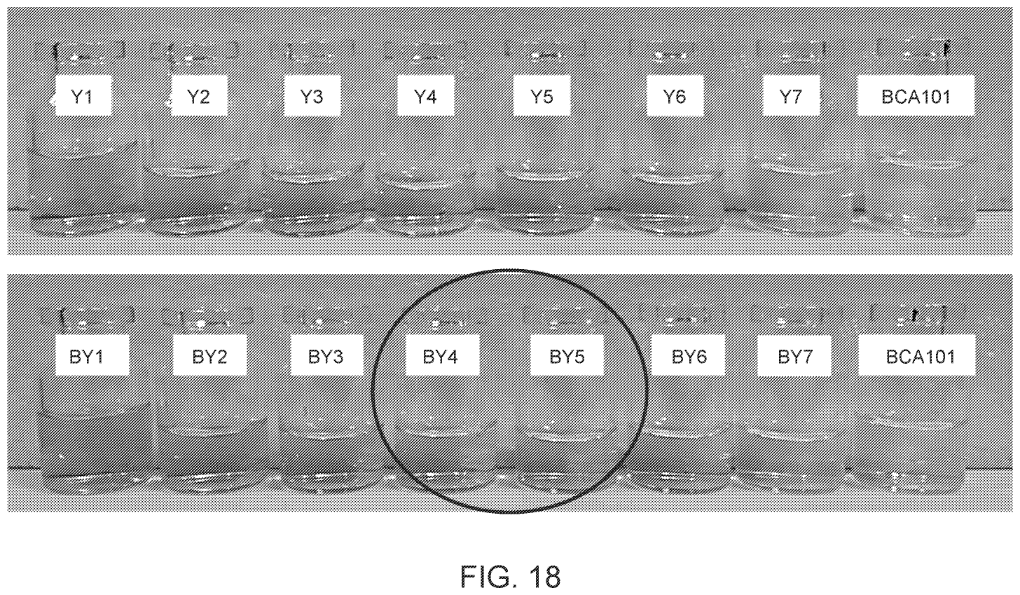

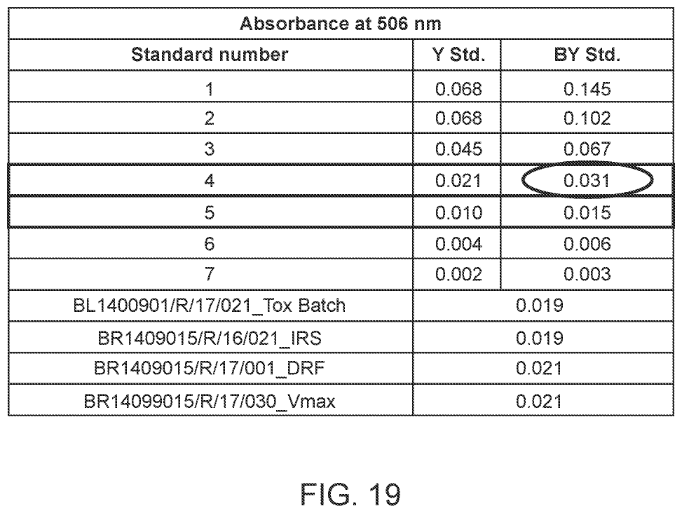

The color of the BCA101 formulation comprising 25 mg/ml BCA101, 0.02% w/v polysorbate 20, 8.0% w/v sucrose, and 10 mM citrate phosphate (citric acid monohydrate 0.573 mg/mL, disodium Hydrogen phosphate dihydrate 1.294 mg/mL) buffer (pH 6.0) was evaluated compared to pharmacopoeial (Ph.Eu.2.2.2) color standard solution (FIG. 18). The standard solution references tables are provided below in Tables 10-12. The absorbance of the BCA101 batches (Toxicology study batch, IRS, and DRF) at 506 nm are shown in FIG. 19.

TABLE 10
Pharmacopoeial (Ph. Eu. 2.2.2) color standard solutions

Volume in milliliters

| | | | | Hydrochloric |
|---|---|---|---|---|
| Standard | Yellow | Red | Blue | Acid (10 g/L |
| Solution | Solution | Solution | Solution | HCL) |
| B (Brown) | 3.0 | 3.0 | 2.4 | 1.6 |
| BY (Brownish- | 2.4 | 1.0 | 0.4 | 6.2 |
| Yellow) | | | | |
| Y (Yellow) | 2.4 | 0.6 | 0.0 | 7.0 |
| GY (Greenish- | 9.6 | 0.2 | 0.2 | 0.0 |
| Yellow) | | | | |
| R (Red) | 1.0 | 2.0 | 0.0 | 7.0 |

TABLE 11
Pharmacopoeial (Ph.Eu.2.2.2) color standard solutions

Volume in milliliters

| Reference | Standard | Hydrochloric Acid |
|---|---|---|
| Solution | Solution Y | (10 g/L HCL) |
| Y₁ | 100.0 | 0.0 |
| Y₂ | 75.0 | 25.0 |
| Y₃ | 50.0 | 50.0 |
| Y₄ | 25.0 | 75.0 |
| Y₅ | 12.5 | 87.5 |
| Y₆ | 5.0 | 95.0 |
| Y₇ | 2.5 | 97.5 |

TABLE 12
Pharmacopoeial (Ph.Eu.2.2.2) color standard solutions

Volume in milliliters

| Reference | Standard | Hydrochloric Acid |
|---|---|---|
| Solution | Solution Y | (10 g/L HCL) |
| BY₁ | 100.0 | 0.0 |
| BY₂ | 75.0 | 25.0 |
| BY₃ | 50.0 | 50.0 |
| BY₄ | 25.0 | 75.0 |
| BY₅ | 12.5 | 87.5 |
| BY₆ | 5.0 | 95.0 |
| BY₇ | 2.5 | 97.5 |

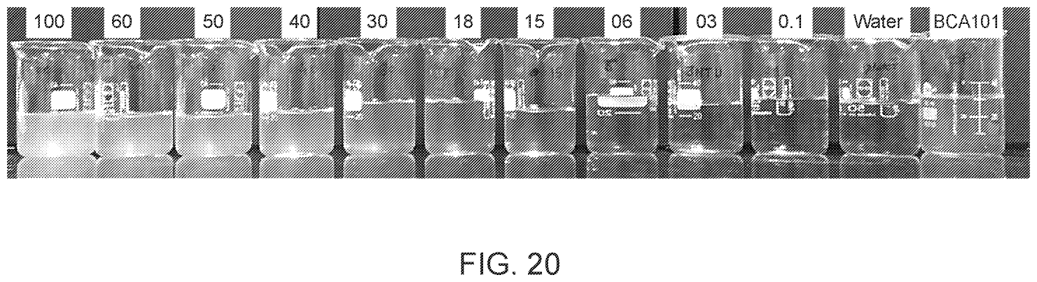

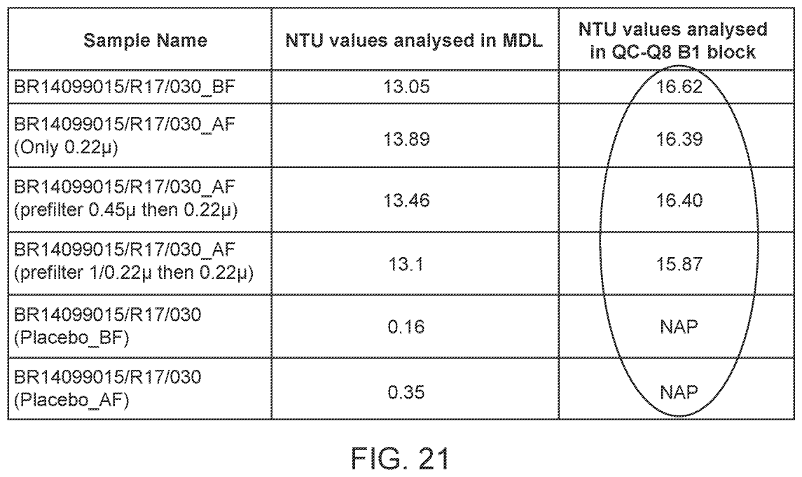

The clarity and degree of opalescence of BCA101 formulation comprising 25 mg/ml BCA101, 0.02% w/v polysorbate 20, 8.0% w/v sucrose, and 10 mM citrate phosphate (citric acid monohydrate 0.573 mg/mL, disodium Hydrogen phosphate dihydrate 1.294 mg/mL) buffer (pH 6.0) was evaluated compared to pharmacopoeial standard (formazin suspensions, Ph.Eu.2.2.1) (FIG. 20). The standard solution references are provided in Table 13 below. The NTU values for the BCA101 samples are presented in FIG. 21. The assay was conducted as per Ph. Eur. 2.2.1, the full contents of which are incorporated by reference herein.

TABLE 13
Pharmacopoeial (Ph.Eu.2.2.2) color standard solutions

| NTU Standard | Observed NTU (analyzed at MDL) |
|---|---|
| 0.1 | 0.07 |
| 3 | 2.57 |
| 6 | 5.20 |
| 15 | 13.37 |
| 18 | 16.57 |
| 30 | 28.11 |
| 40 | 37.50 |
| 50 | 47.70 |
| 60 | 57.00 |
| 100 | 97.01 |
| 750 | Not Applicable (NAP) |
| 1000 | NAP |

### Long Term Stability Study

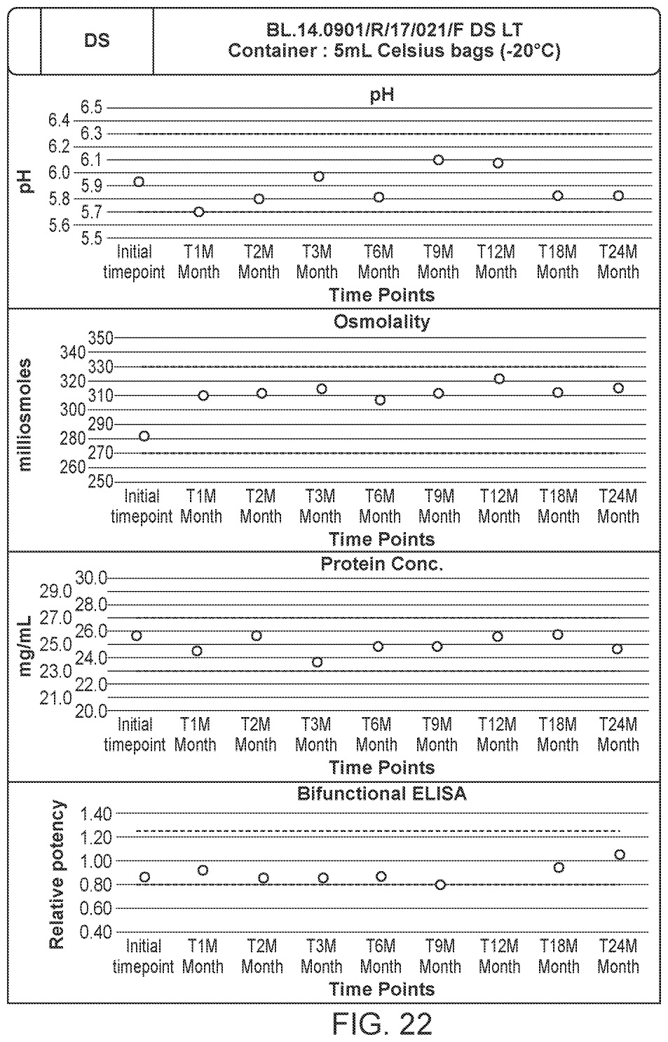

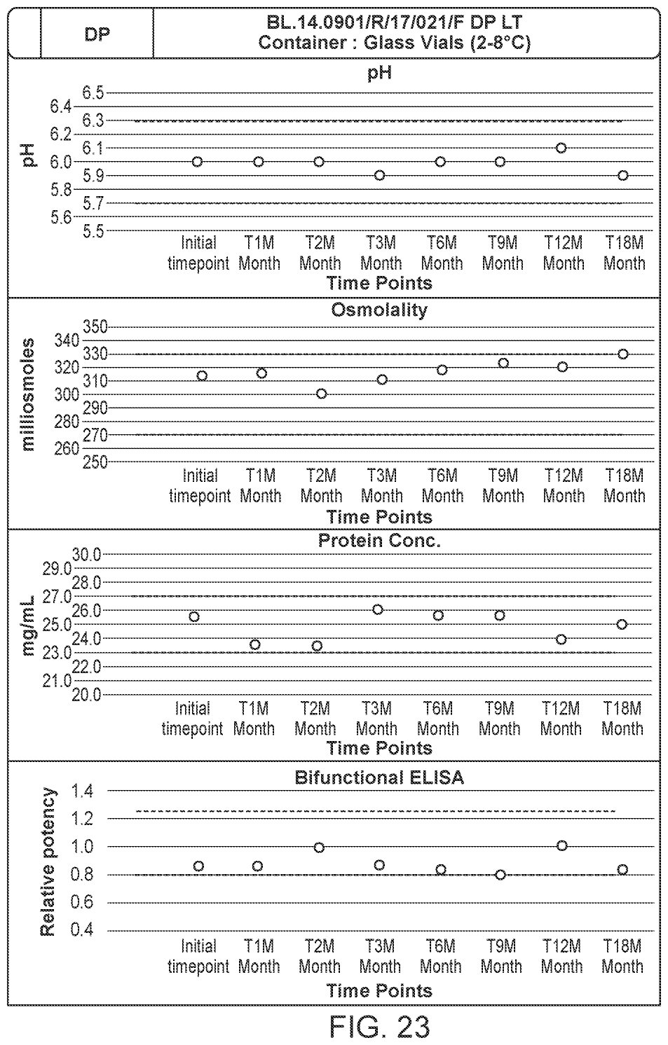

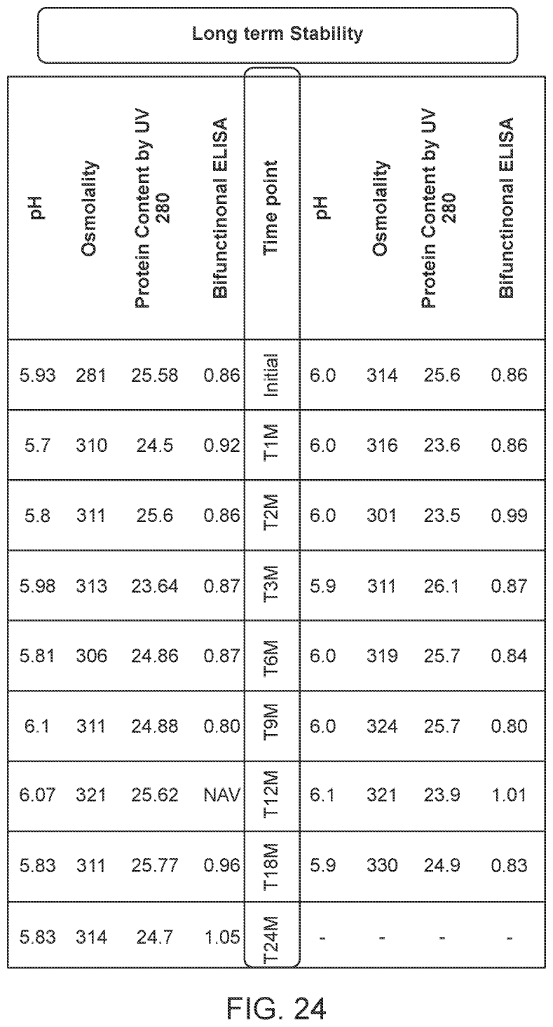

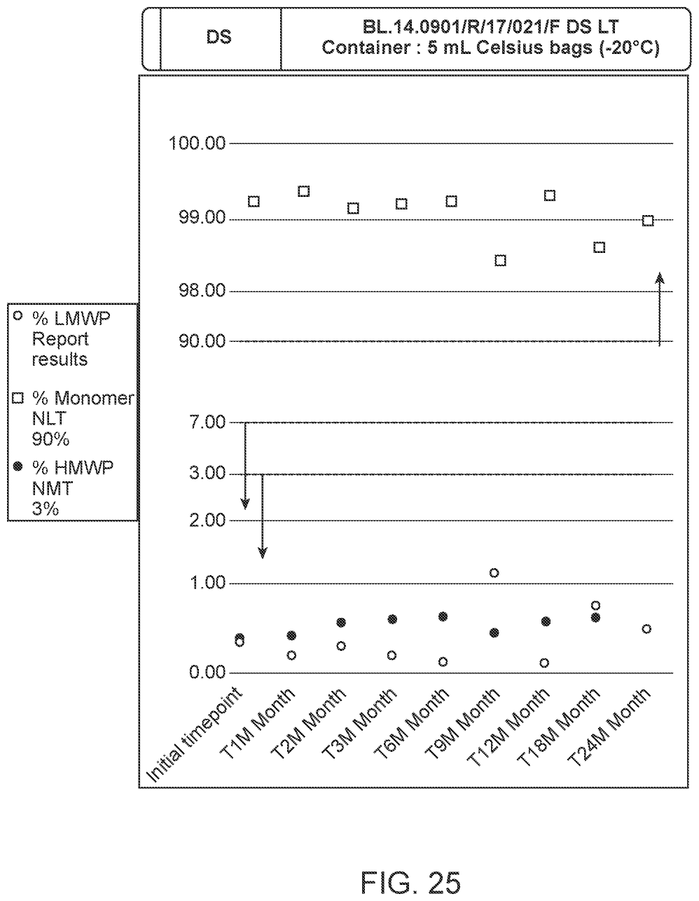

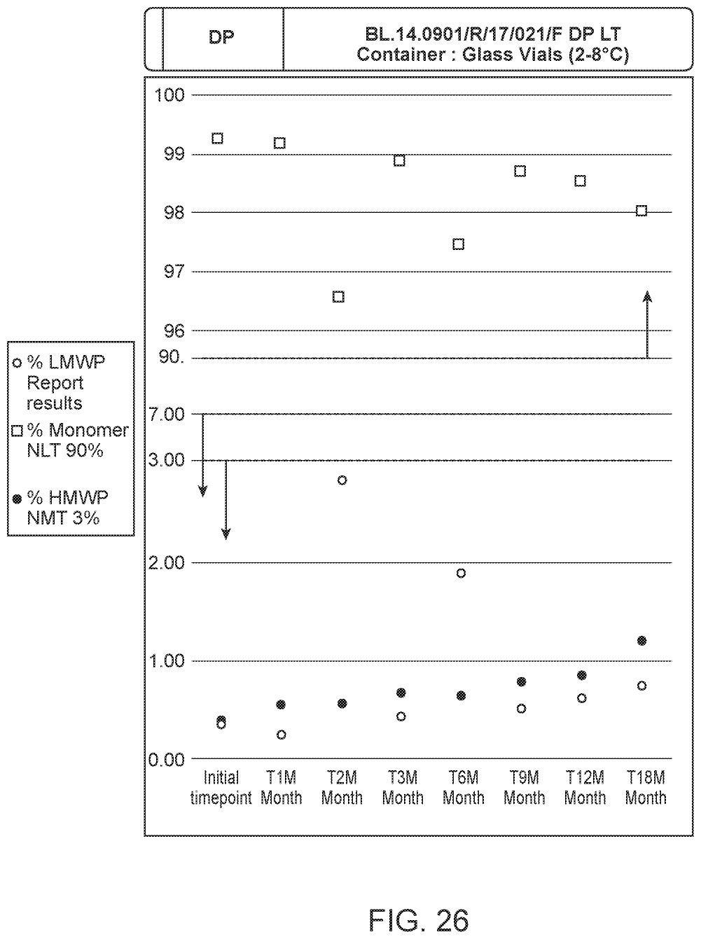

The stability of BCA101 in the formulation comprising 25 mg/ml BCA101, 0.02% w/v polysorbate 20, 8.0% w/v sucrose, and 10 mM citrate phosphate (citric acid monohydrate 0.573 mg/mL, disodium Hydrogen phosphate dihydrate 1.294 mg/mL) buffer (pH 6.0) was evaluated for a toxicology study batch if the drug substance (DS) in 5 mL Celsius bags at 2-20° C. (FIG. 22 and FIG. 25) and drug product (DP) in glass vials at 2-8° C. (FIG. 23 and FIG. 26). The pH, osmolality, protein concentration, and functionality of both arms the BCA101 fusion protein (via bifunctional ELISA measuring the ability to bind hEGFR and hTGFβ) drug substance (FIG. 22) and drug product (FIG. 23) were evaluated across 24 months, with data points taken at the initial timepoint, 1 month, 2 months, 3 months, 6 months, 9 months, 12 months, 18 months, and 24 months. A graphical comparison of the results from the long stability study between the drug substance and drug product is shown in FIG. 24. Briefly, to carry out the bifunctional ELISA, recombinant hEGFR Fc coated plates were blocked and subsequently incubated with BCA101 for about 1 hour, followed by incubation with recombinant hTGFβ1. hTGFβ1 bound to hTGFβRII ECD moiety of BCA101 was then detected with biotinylated anti-hTGFβ1 antibody followed by streptavidin-HRP. Thereby, the signal will be obtained only when both arms are intact.

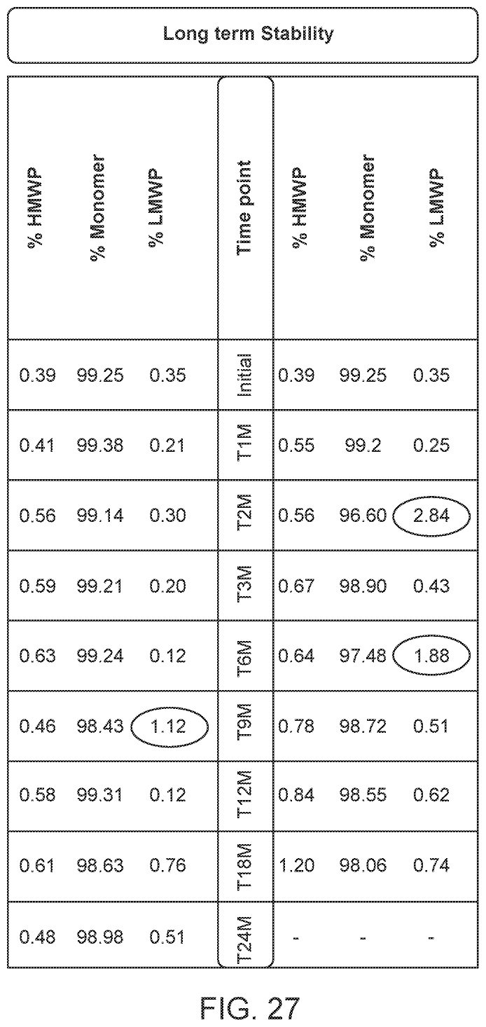

The percent HMWP, percent monomer, and percent LMWP were also evaluated for a toxicology study batch if the drug substance (DS) in 5 mL Celsius bags at 2-20° C. (FIG. 25) and drug product (DP) in glass vials at 2-8° C. (FIG. 26). A graphical comparison of the results from the long stability study between the drug substance and drug product is shown in FIG. 27.

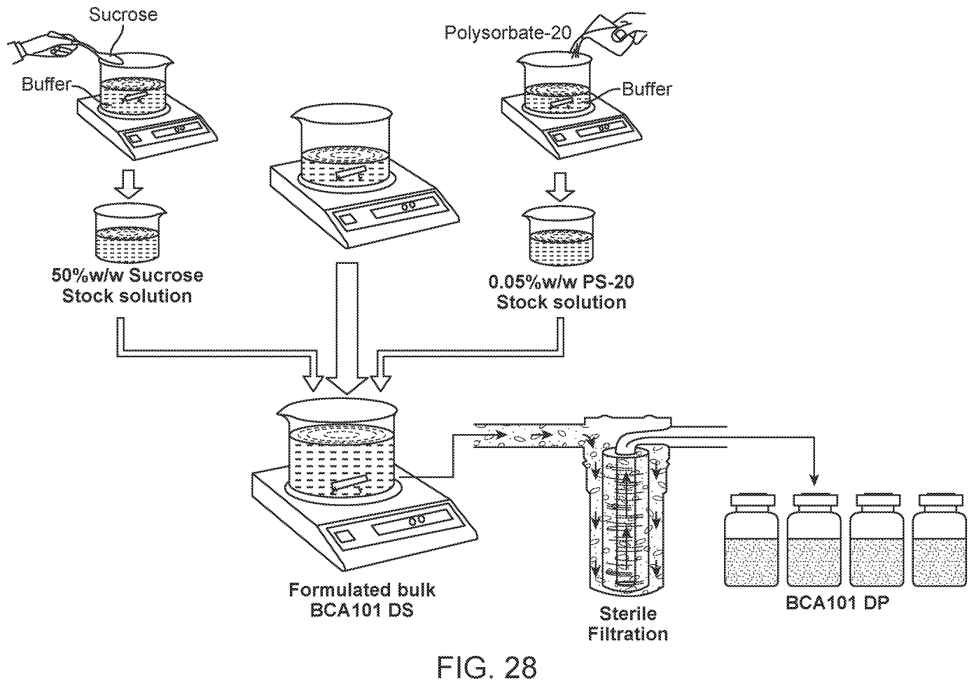

A graphical representation of an exemplary formulation process described in the above example is shown in FIG. 28.

### Example 2. Long Term Storage Stability of BCA101 Drug Substance (DS)

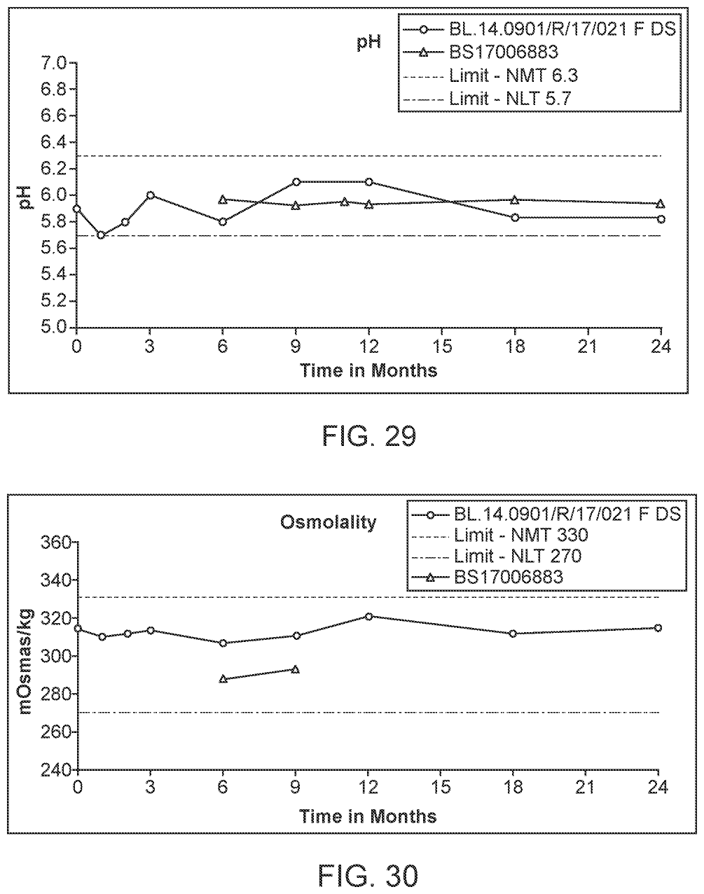

The objective of this study was to evaluate the long-term storage stability of BCA101 drug substance (DS) over 24 months at −20±5° C. Two different batches of BCA100 DS (BL.14.0901/R/17/021 F DS (an R&D toxicology batch) and BS17006883 (a GMP development batch)) each comprising 25 mg/ml BCA101, 0.02% w/v polysorbate 20, 8.0% w/v sucrose, and 10 mM citrate phosphate (citric acid monohydrate 0.573 mg/mL, disodium Hydrogen phosphate dihydrate 1.294 mg/mL) buffer (pH 6.0) stored either in a Flexboy bag (BL.14.0901/R/17/021 F DS) or Celsius FFT or Celsius Pak bags (BS17006883) at −20±5° C. were evaluated. The pH, osmolality, protein concentration, % monomer, % high molecular weight protein (HMWP), % low molecular weight protein (LMWP), functionality of both arms the BCA101 fusion protein (via bifunctional ELISA measuring the ability to bind hEGFR and hTGFβ), visual description, color, clarity, protein concentration, purity by SEC-HPLC and RP-HPLC, inhibition of EGFR expressing cell proliferation, bacterial endotoxin, and bioburden were evaluated across 24 months, with data points generally taken at the initial timepoint, 1 month, 2 months, 3 months, 6 months, 9 months, 12 months, 18 months, and 24 months (except as noted below in Tables 14 and 15). Tables 14 and 15 provide a compilation of the stability data for both batches of BCA101 DS. A description of each of the individual stability tests and trend data are provided below and in FIGS. 29-39.

Table 14 below, provides a compilation of the long-term stability data for the BCA101 DS batch BL.14.0901/R/17/021 F DS stored at −20±5° C., with the individual stability tests described in further detail below.

TABLE 14
Compilation of the long-term stability data for the BCA101 DS BL.14.0901/R/17/021 F DS batch stored at −20 ± 5° C.

| Stability | | Acceptance | Stability Data Trend from Date of Inception (Months) | | | | | | | | |
|---|---|---|---|---|---|---|---|---|---|---|---|
| Tests | Parameters | Criteria | TO M | T1 M | T2 M | T3 M | T6 M | T9 M | T12 M | T18 M | T24 M |
| Purity by | % Monomer | NLT 93% | 99.25 | 99.38 | 99.14 | 99.21 | 99.24 | 98.43 | 99.31 | 98.63 | 98.98 |
| SEC-HPLC | % HMWP | NMT 3.0% | 0.39 | 0.41 | 0.56 | 0.59 | 0.63 | 0.46 | 0.58 | 0.61 | 0.48 |
| | % LMWP | Report | 0.35 | 0.21 | 0.30 | 0.20 | 0.12 | 1.12 | 0.12 | 0.76 | 0.55 |
| | | Result | | | | | | | | | |
| UV-280 | Protein | 25 ± 2 | 25.6 | 24.5 | 25.6 | 23.6 | 25.1 | 24.9 | 25.6 | 25.8 | 24.7 |
| | Conc. | mg/mL | | | | | | | | | |
| | (mg/ml) | | | | | | | | | | |
| pH | pH | 6.0 ± 0.3 | 5.90 | 5.70 | 5.80 | 6.00 | 5.80 | 6.10 | 6.10 | 5.83 | 5.82 |
| Osmolality | Osmolality | 300 ± 30 | 314 | 310 | 311 | 313 | 306 | 311 | 321 | 311 | 314 |
| | of solution | mOsmol/kg | | | | | | | | | |
| Bifunctional | Relative | 0.80-1.25 | 0.86 | 0.92 | 0.86 | 0.87 | 0.87 | 0.80 | 0.76 | 0.96 | 1.05 |
| Assay | Potency | | | | | | | | | | |

NLT = Not less than; NMT = Not more than; SEC-HPLC = Size exclusion chromatography high performance liquid chromatography; UV-280 = Ultraviolet Light Absorbance at 280 nm.

Table 15 below, provides a compilation of the long-term stability data for the BS17006883 of BCA101 DS (GMP batch) stored at −20±5° C., with the individual stability tests described in further detail below.

TABLE 15
Compilation of the long-term stability data for the BS17006883
BCA101 DS batch stored at −20 ± 5° C.

| Test | *Acceptance | | | | | | |
|---|---|---|---|---|---|---|---|
| Method | Criteria | *T6 M | T9 M | T11 M | T12 M | T18 M | T24 M |
| Description | A clear to | Complies | Complies | Complies | Complies | Complies | Complies |
| | opalescent | | | | | | |
| | liquid, free | | | | | | |
| | from foreign | | | | | | |
| | matter | | | | | | |
| Clarity | Opalescent | Complies | Complies | Complies | Complies | Complies | Complies |
| | should not be | | | | | | |
| | more than Ph. | | | | | | |
| | Eur. Reference | | | | | | |
| | standard III | | | | | | |
| | (NTU value | | | | | | |
| | should NMT | | | | | | |
| | 18 NTU) | | | | | | |
| Color | Not more | Complies | Complies | Complies | Complies | Complies | Complies |
| | intensely | | | | | | |
| | colored than | | | | | | |
| | Ph. Eur | | | | | | |
| | reference | | | | | | |
| | standard BY4 | | | | | | |
| pH | 6.00 ± 0.30 | 5.97 | 5.92 | 5.95 | 5.93 | 5.96 | 5.94 |
| Osmolality | 300 ± 30 | 288 | 293 | Do not | Do not | Do not | Do not |
| | mOsmol/kg | | | test | test | test | test |
| Protein | 25.0 ± 2.0 | 24.7 | 24.9 | 24.9 | 24.4 | 24.7 | 25.4 |
| Concentration | mg/mL | | | | | | |
| Purity by | HMWP %: | 0.86 | 0.67 | 0.70 | 0.60 | 0.80 | 0.70 |
| SEC-HPLC | NMT 3.0% | | | | | | |
| | LMWP %: | 0.36 | 1.02 | 1.20 | 1.00 | 0.70 | 0.30 |
| | Report Results | | | | | | |
| | Monomer: | 98.8 | 98.3 | 98.1 | 98.4 | 98.6 | 99.0 |
| | NLT 93.0% | | | | | | |
| Purity by | Main Peak: | 82.44 | 80.60 | 85.6 | 84.7 | 81.1 | 82.2 |
| RP-HPLC | NLT 70.0% | | | | | | |
| | Total Pre-Peak: | 4.45 | 4.33 | 3.3 | 2.9 | 4.5 | 4.3 |
| | NMT 6.0% | | | | | | |
| | Total Post-Peak: | 13.12 | 15.09 | 11.1 | 12.5 | 14.4 | 13.5 |
| | NMT 24.0% | | | | | | |
| Bi-Functional | Relative Potency: | 0.84 | 0.94 | 0.99 | 0.93 | 0.98 | 0.97 |
| ELISA | 0.80 to 1.25 of | | | | | | |
| | ref standard | | | | | | |
| Inhibition of | Relative Potency: | 0.94 | 0.90 | 0.98 | 1.05 | 0.99 | 1.04 |
| Proliferation | 0.80 to 1.25 of | | | | | | |
| | ref standard | | | | | | |
| Bacterial | NMT 0.25 | Complies | Do not | Complies | Complies | Do not | Complies |
| Endotoxin | EU/mg | | test | | | test | |
| Bioburden | NMT 10 | Complies | Do not | Complies | Complies | Do not | Complies |
| | CFU/100 mL | | test | | | test | |

NLT = Not less than; NMT = Not more than; NTU = Nephelometric turbidity units; SEC-HPLC = Size exclusion chromatography high performance liquid chromatography; RP-HPLC = Reversed phase high pressure liquid chromatography; UV-280 = Ultraviolet Light Absorbance at 280 nm

### Visual Observations

Visual observations is a qualitative evaluation of the description, clarity, and color of the manufactured drug substance. Based on the manufacturing data the following specifications were set for BCA101 DS: Description: the absence of interfering foreign particles is set to “A clear opalescent liquid, free from foreign matter”; Clarity: “Opalescent should not be more than Ph. Eur. Reference standard III (NTU value should NMT 18 NTU)”; and Color: “Not more intensely colored than Ph. Eur reference standard BY4”. The BS17006883 BCA101 DS batch was tested for visual observations and shown to comply with each of the specifications up to 24 months from the date of manufacturing (see Table 15). The BL.14.0901/R/17/021 F DS BCA101 DS batch was not tested for visual observations.

### pH Range

The pH range of BCA101 DS was set at pH 6.00±0.30 units, based on optimal stability for BCA101 DS. Any change in pH is an indication of degradation of one or more of the components in the formulation. Both BCA101 DS batches were tested and determined to comply with the pH specification for up to 24 months from the date of manufacture. From the trend analysis for DS batches, there is no appreciable change in pH, as shown in Tables 14 and 15, and FIG. 29.

### Osmolality

Osmolality is one of the parameters, which needs to be controlled for parenteral formulations close to that of blood plasma in order to avoid adverse reactions associated with injecting hypo/hypertonic DS during administration. The iso-osmotic concentration for BCA101 was majorly achieved using sucrose in the formulation and the osmolality specification was set at 270 to 330 mOsmol/kg for the BCA101 DS. Any change in sucrose concentration during the stability period could lead to a change in osmolality indicating an impact on the stability of the substance. As shown in FIG. 30 and Tables 14 and 15, the osmolality of the BL.14.0901/R/17/021 F DS BCA101 DS batch was within the specification for up to 24 months, and the BS17006883 BCA101 DS batch was also determined to be within the specification measured at the 6- and 9-month time points. The osmolality of the BS17006883 BCA101 DS batch was not tested past the 9-month time point.

### Protein Concentration

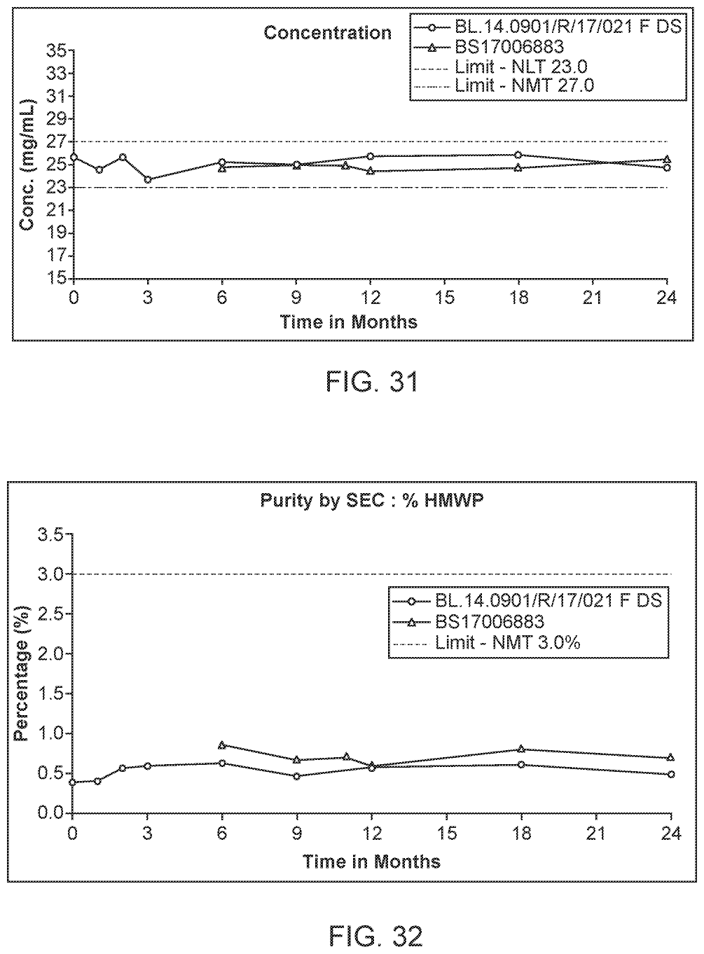

Protein concentration provides information about the quantity of the DS in the sample. Based on developmental data, limits for BCA101 DS protein concentration were set at 25.00±2.00 mg/mL. As shown in FIG. 31 and Tables 14 and 15, both of the BCA101 DS batches complied with the protein concentration specification up to 24 months from the date of manufacture.

### Bioburden and Bacterial Endotoxin Test (BET)

The acceptance criteria for BET and bioburden for BCA101 DS were set at not more than (NMT) 0.25 EU/mg and not more than (NMT) 10 CFU/100 mL, respectively. As shown in Table 15, the BS17006883 BCA101 DS batch met both the BET and bioburden specifications up to 24 months from the date of manufacture. The BL.14.0901/R/17/021 F DS BCA101 DS batch was not tested for bioburden or BET.

### Purity by SEC-HPLC

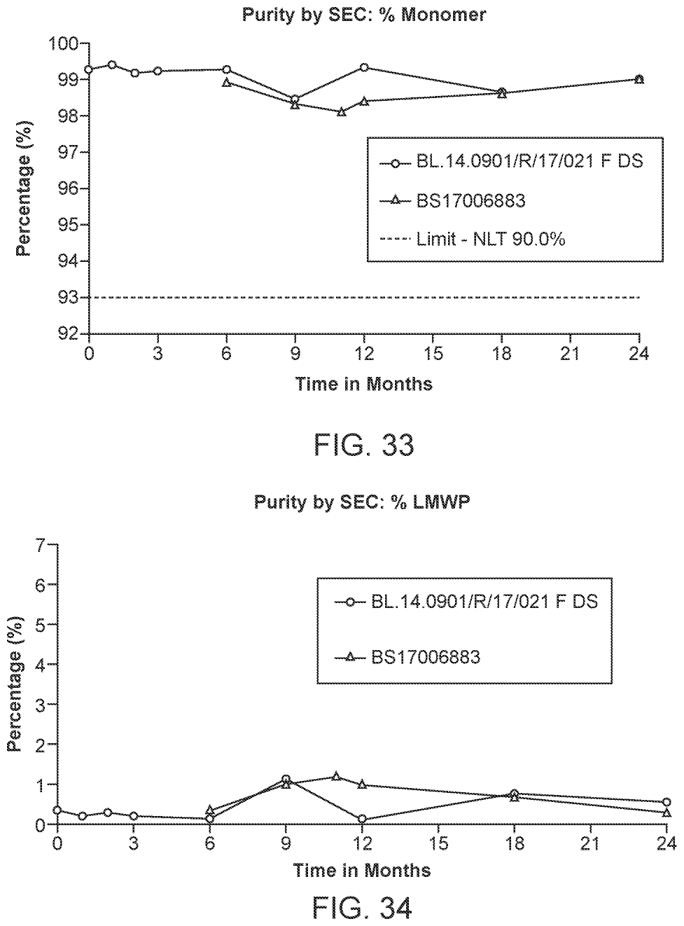

SEC-HPLC provides information about monomer content and related HMWPs. The acceptance criteria for BCA101 DS were set as follows: Monomer % NLT 93.00%, HMWP % NMT 3.00%, and LMWP report result. As shown in FIG. 32 (% HMWP), FIG. 33 (% monomer), and FIG. 34 (% LMWP), and Tables 14 and 15, the SEC-HPLC purity results complied with the specification limits for both BCA101 DS batches up to the 24 months from the date of manufacture.

### Purity by RP-HPLC

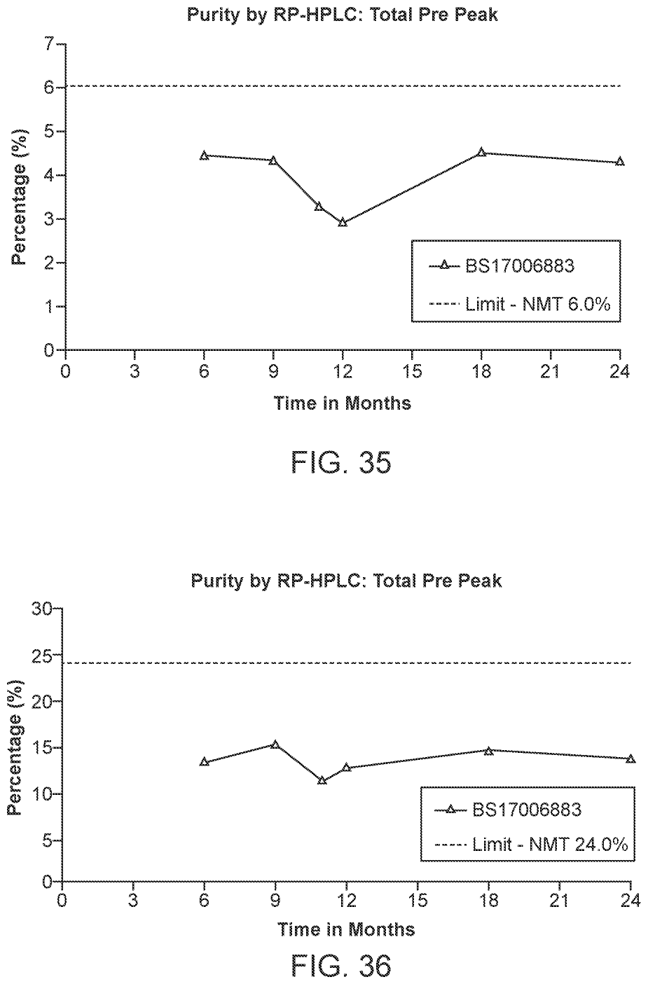

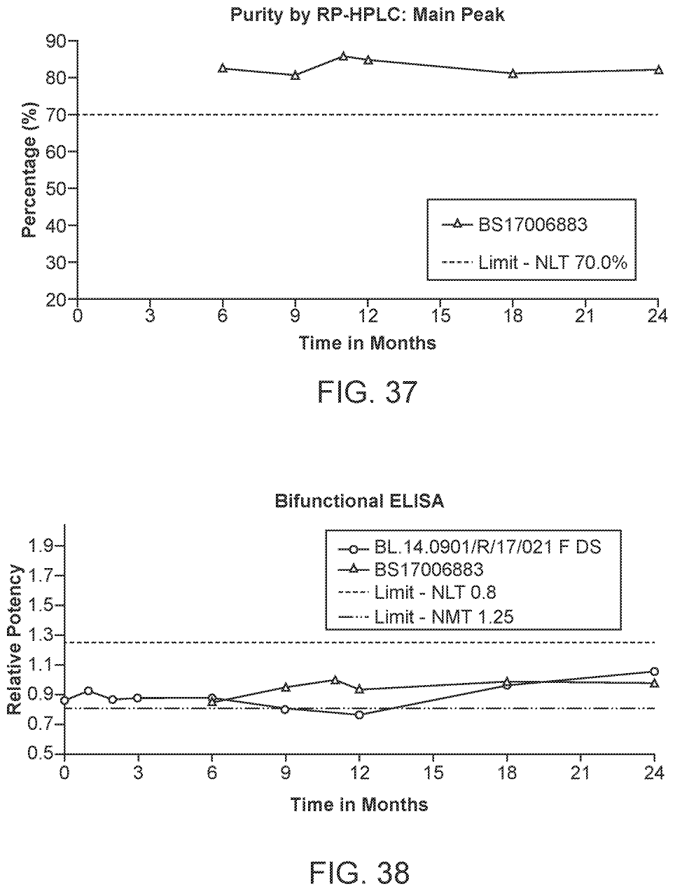

RP-HPLC provides information about purity with respect to hydrophobic variants. The acceptance criteria for BCA101 DS were set as follows: Total Main Peak NLT 70.0%, Total Post Peaks NMT 24%, and Total Pre-Peaks NMT 6%. As shown in FIG. 35 (total pre peak), FIG. 36 (total post peak), and FIG. 37 (% main peak), and Tables 14 and 15, the RP-HPLC purity results complied with the specification limits for the BS17006883 BCA101 DS batch up to 24 months from the date of manufacture. The BL.14.0901/R/17/021 F DS BCA101 DS batch was not tested by RP-HPLC.

### Bi-Functional ELISA

A bi-functional ELISA was used to determine the simultaneous binding efficacy of BCA101 to the EGF receptor (EGFR) and TGFβ1 ligand. Briefly, recombinant hEGFR Fc coated plates were blocked and subsequently incubated with BCA101 for about 1 hour, followed by incubation with recombinant hTGFβ1. hTGFβ1 bound to hTGFβRII ECD moiety of BCA101 was then detected with biotinylated anti-hTGFβ1 antibody followed by streptavidin-HRP. Thereby, the signal will be obtained only when both antigen binding arms of BCA101 (binding EGFR and binding TGFβ1 ligand) are intact. The assay acceptance criterion was set to an average relative potency of 0.80 to 1.25 with respect to reference standard.

As shown in FIG. 38, the relative potency of both BCA101 DS batches was well within the assay acceptance criteria until the last time-point tested, 24 months from date of manufacture.

### Inhibition of Proliferation (IOP) Assay

An inhibition of proliferation (TOP) assay was used to determine the ability of BCA101 DS to inhibit FaDu cancer cell growth by binding to its target EGFR. This assay provides a method to determine the number of viable cells in culture by quantitating the amount of ATP present. The read out was based on luminescence by mono-oxygenation of luciferin which is catalyzed by luciferase in the presence of Mg2+, and ATP released by viable cells. The assay acceptance criterium was set to an average relative potency of 0.80 to 1.25 with respect to reference standard.

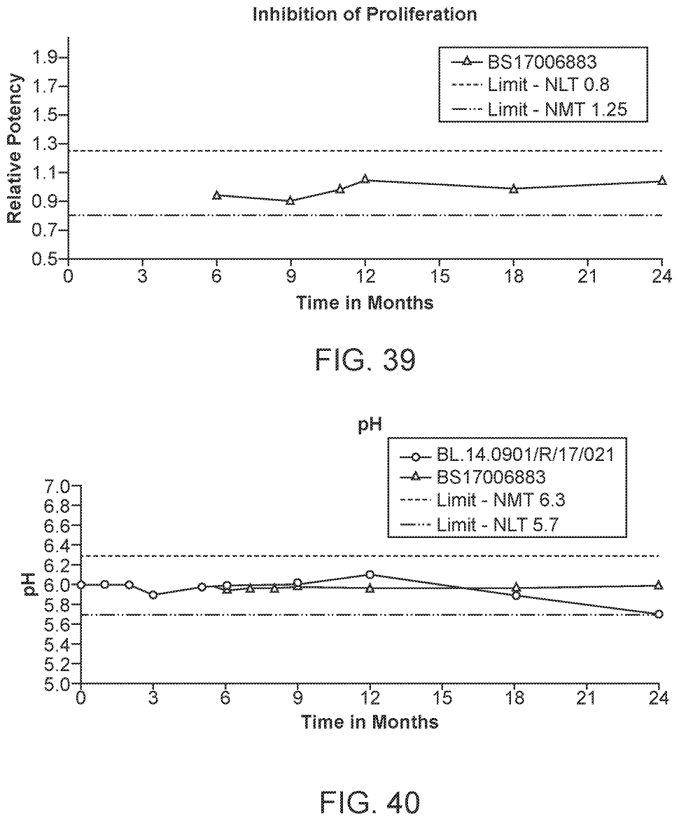

As shown in FIG. 39 (and Table 14), the BS17006883 BCA101 DS batch was determined to comply with the IOP specification of 0.80 to 1.25 up to 24 months from the date of manufacture. The BL.14.0901/R/17/021 F DS BCA101 DS batch was not tested by TOP.

### Conclusions

The above data shows that multiple BCA101 drug substance batches stored at −20±5° C. up to 24 months from the date of manufacture met the various and comprehensive critical quality attribute (release parameters) specifications. Based on the stability trend analysis, no considerable change in pH, osmolality, SEC-HPLC, protein content/concentration, RP-HPLC or functionality were observed. The data obtained from the R&D (BL.14.0901/R/17/021 F DS) and developmental GMP batch (BS17006883) indicates physico-chemical and functional stability of the BCA101 drug substance formulation over 24 months from the date of manufacture when stored at −20±5° C. in either Flexboy or Celsius FFT/Celsius Pak bags.

### Example 3. Long Term Storage Stability of BCA101 Drug Product (DP)

The objective of this study was to evaluate the long-term storage stability of BCA101 drug product (DP) stored over 24 months at 5±3° C. Two different batches of BCA100 DP (BL.14.0901/R/17/021 F DP (an R&D batch) and BS18002245 (a GMP development batch)) comprising 25 mg/ml BCA101, 0.02% w/v polysorbate 20, 8.0% w/v sucrose, and 10 mM citrate phosphate (citric acid monohydrate 0.573 mg/mL, disodium Hydrogen phosphate dihydrate 1.294 mg/mL) buffer (pH 6.0) stored in10R USP type I Clear glass vials at 5±3° C. were evaluated. The pH, osmolality, protein concentration, % monomer, % high molecular weight protein (HMWP), % low molecular weight protein (LMWP), functionality of both antigen binding arms the BCA101 fusion protein (via bifunctional ELISA measuring the ability to bind hEGFR and hTGFβ), purity (by SEC-HPLC and RP-HPLC), inhibition of EGFR expressing cell proliferation, visual description, clarity, color, extractable volume, seal integrity, sub-visible particulate matter, bacterial endotoxin, and sterility were evaluated across 24 months, with data points generally taken at the initial timepoint, 1 month, 2 months, 3 months, 6 months, 9 months, 12 months, 18 months, and 24 months (except as noted in Tables 16 and 17). Tables 16 and 17 provide a compilation of the stability data for the first and second batch of BCA101 DP. A description of each of the individual stability tests and trend data are provided below and in FIGS. 40-51.

Table 16 below, provides a compilation of the long-term stability data for the BL.14.0901/R/17/021 F DP BCA101 DP batch (R&D batch) stored at 5±3° C., with the individual stability tests described in further detail below.

TABLE 16
Compilation of the long-term stability data for the BL.14.0901/R/17/021 F DP BCA101 DP batch stored at 5 ± 3° C.

| Stability | | Acceptance | Stability Data Trend from Date of Inception (Months) | | | | | | | | |
|---|---|---|---|---|---|---|---|---|---|---|---|
| Tests | Parameters | Criteria | TO M | T1 M | T2 M | T3 M | T6 M | T9 M | T12 M | T18 M | T24 M |
| Purity by | % Monomer | NLT 93% | 99.25 | 99.2 | 96.6 | 98.9 | 97.48 | 98.72 | 98.55 | 98.06 | 98.00 |
| SEC-HPLC | % HMWP | NMT 3.0% | 0.39 | 0.55 | 0.56 | 0.67 | 0.64 | 0.78 | 0.84 | 1.2 | 1.03 |
| | % LMWP | Report | 0.35 | 0.25 | 2.84 | 0.43 | 1.88 | 0.51 | 0.62 | 0.74 | 0.96 |
| | | Result | | | | | | | | | |
| UV-280 | Protein | 25 ± 2 | 25.6 | 23.6 | 23.5 | 26.1 | 25.7 | 25.7 | 23.9 | 24.97 | 24.1 |
| | Conc. | mg/mL | | | | | | | | | |
| | (mg/ml) | | | | | | | | | | |
| pH | pH of the | 6.0 ± 0.3 | 6.0 | 6.0 | 6.0 | 5.9 | 6.0 | 6.0 | 6.1 | 5.9 | 5.7 |
| | drug product | | | | | | | | | | |
| | solution | | | | | | | | | | |
| Osmolality | Osmolality | 300 ± 30 | 314 | 316 | 301 | 311 | 319 | 324 | 321 | 330 | 322 |
| | of solution | mOsmo/kg | | | | | | | | | |
| Bifunctional | Average | 0.80-1.25 | 0.86 | 0.86 | 0.99 | 0.87 | 0.84 | 0.80 | 1.01 | 0.83 | 0.85 |
| Assay | Relative | | | | | | | | | | |
| | Potency | | | | | | | | | | |
| IOP | Average | 0.80-1.25 | NAP | NAP | NAP | NAP | NAP | NAP | NAP | NAP | NAP |
| | Relative | | | | | | | | | | |
| | Potency | | | | | | | | | | |
| δPurity by | Total Pre-Peak | NMT6.0% | 3.91 | 3.99 | 4.02 | 6.99* | NAP | 4.59 | 4.64 | 4.17 | 4.98 |
| RP-HPLC | Main Peak | NLT70.0% | 76.83 | 76.46 | 76.53 | 42.54* | | 75.99 | 76.42 | 76.97 | 74.69 |
| | Total Post-Peak | NMT24.0% | 19.26 | 19.55 | 19.45 | 50.48* | | 19.43 | 18.92 | 18.86 | 20.33 |

NLT = Not less than; NMT = Not more than; SEC-HPLC = Size exclusion chromatography high performance liquid chromatography; RP-HPLC = Reversed phase high pressure liquid chromatography; UV-280 = Ultraviolet Light Absorbance at 280 nm; NAP = Not applicable.
^{δ}The stability samples up to T12, were stored at −80° C. and the test for purity by RP-HPLC was carried out at a later date.
*The out-of-t rend RP-HPLC data for T3 M is due to assay-related deviation as the data from subsequent time points, including the longest available data for T24 M, are within the specification. This suggests that the product quality is within the specified limits and the observed deviation at T3 M is an anomaly.

Table 17 below, provides a compilation of the long-term stability data for the BS18002245 BCA101 DS batch (GMP batch) stored at 5±3° C., with the individual stability tests described in further detail below.

TABLE 17
Compilation of the long-term stability data for the BS18002245 BCA101 DP batch stored at 5 ± 3° C.

| Test | Acceptance | | | | | | | | |
|---|---|---|---|---|---|---|---|---|---|
| Method | Criteria | *T5 M | T6 M | T7 M | T8 M | T9 M | T12 M | T18 M | T24 M |
| Description | A clear to | Complies | Complies | Complies | Complies | Complies | Complies | Complies | Complies |
| by visual | opalescent | | | | | | | | |
| observation | liquid, free | | | | | | | | |
| | from foreign | | | | | | | | |
| | matter | | | | | | | | |
| Clarity | Opalescent | Complies | Complies | Complies | Complies | Complies | Complies | Complies | Complies |
| | should not be | | | | | | | | |
| | more than Ph. | | | | | | | | |
| | Eur. Reference | | | | | | | | |
| | standard III | | | | | | | | |
| | (NTU value | | | | | | | | |
| | should NMT | | | | | | | | |
| | 18 NTU) | | | | | | | | |
| Color | Not more | Complies | Complies | Complies | Complies | Complies | Complies | Complies | Complies |
| | intensely | | | | | | | | |
| | colored than | | | | | | | | |
| | Ph. Eur | | | | | | | | |
| | reference | | | | | | | | |
| | standard BY4 | | | | | | | | |
| pH | 5.70-6.30 | 5.99 | 5.95 | 5.96 | 5.96 | 5.98 | 5.96 | 5.97 | 6.0 |
| Protein | 23.0-27.0 | 24.3 | 23.8 | 24.6 | 25.1 | 24.2 | 25.1 | 25.6 | 24.6 |
| Concentration | mg/mL | | | | | | | | |
| Extractable | NLT 10.0 mL | 10.0 | 10.0 | 10.3 | 10.3 | 10.3 | 10.5 | 11.0 | 11.0 |
| Volume | | | | | | | | | |
| Seal | None of the | Complies | Complies | Complies | Complies | Complies | Complies | Complies | Complies |
| Integrity | vials tested | | | | | | | | |
| Test | should contain | | | | | | | | |
| | any trace of | | | | | | | | |
| | colored | | | | | | | | |
| | solution | | | | | | | | |
| Sub Visible | Particles per | 949 | 1366 | 3675 | 854 | 595 | 1637 | 14707 | 13173 |
| Particulate | container. | | | | | | | | |
| Matter > | Report Value. | | | | | | | | |
| or = 2 μm | | | | | | | | | |
| Sub Visible | Particles per | 371 | 385 | 750 | 257 | 106 | 508 | 5046 | 4057 |
| Particulate | container. | | | | | | | | |
| Matter > | Report Value | | | | | | | | |
| or = 5 μm | | | | | | | | | |
| Sub Visible | NMT 6000 | 125 | 67 | 134 | 51 | 7 | 32 | 358 | 251 |
| Particulate | particles per | | | | | | | | |
| Matter > | container | | | | | | | | |
| or = 10 μm | | | | | | | | | |
| Sub Visible | NMT 600 | 3 | 5 | 1 | 0 | 0 | 1 | 0 | 4 |
| Particulate | particles per | | | | | | | | |
| Matter > | container | | | | | | | | |
| or = 25 um | | | | | | | | | |
| Purity by | HMWP %: | 0.9 | 0.8 | 0.9 | 0.9 | 0.9 | 1.0 | 1.0 | 1.1 |
| SEC-HPLC | NMT 3.0% | | | | | | | | |
| | LMWP %: | 1.2 | 1.5 | 0.8 | 1.2 | 1.1 | 1.0 | 1.0 | 1.4 |
| | Report Results | | | | | | | | |
| | Monomer: | 97.9 | 97.7 | 98.3 | 98 | 98 | 98 | 98 | 97.5 |
| | NLT 93.0% | | | | | | | | |
| Purity by | Main Peak: | 85.5 | 80.3 | 81.5 | 81.9 | 81.7 | 80.4 | 80.5 | 80.1 |
| RP-HPLC | NLT 70.0% | | | | | | | | |
| | Total Pre-Peak: | 3.5 | 4.8 | 4.1 | 4.1 | 4.1 | 4.7 | 4.5 | 4.4 |
| | NMT 6.0% | | | | | | | | |
| | Total Post-Peak: | 11.0 | 14.9 | 14.4 | 14.0 | 14.2 | 14.9 | 15 | 15.4 |
| | NMT 24.0% | | | | | | | | |
| Bi-Functional | Average Relative | 0.94 | 0.94 | 1.02 | 1.00 | 0.88 | 0.99 | 1.00 | 0.93 |
| ELISA | Potency: 0.80 | | | | | | | | |
| | to 1.25 of ref | | | | | | | | |
| | standard | | | | | | | | |
| Inhibition of | Average Relative | 0.96 | 0.97 | 0.97 | 0.94 | 0.96 | 1.05 | 1.06 | 1.11 |
| Proliferation | Potency: 0.80 | | | | | | | | |
| | to 1.25 of ref | | | | | | | | |
| | standard | | | | | | | | |
| Bacterial | NMT 0.25 | Complies | Do not | Do not | Do not | Do not | Complies | Do not | Complies |
| Endotoxin | EU/mg | | test | test | test | test | | test | |
| Sterility | Absence of | Complies | Do not | Do not | Do not | Do not | Complies | Do not | Complies |
| | microbial | | test | test | test | test | | test | |
| | growth | | | | | | | | |

NLT = Not less than; NMT = Not more than; NTU = Nephelometric turbidity units; SEC-HPLC = Size exclusion chromatography high performance liquid chromatography; RP-HPLC = Reversed phase high pressure liquid chromatography; UV-280 = Ultraviolet Light Absorbance at 280 nm.
*The batch was incepted into long-term stability at 5 ± 3° C. post manufacturing but the earliest time-point data available was for T5 M.

### Visual Observations

Visual observations is a qualitative evaluation of description, clarity, and color of the manufactured drug product. Based on manufacturing data, the specifications were set as follows: Description: the absence of interfering foreign particles was set to “A clear opalescent liquid, free from foreign matter”; Clarity: “Opalescent should not be more than Ph. Eur. Reference standard III (NTU value should NMT 18 NTU)”; and Color: “Not more intensely colored than Ph. Eur reference standard BY4”. The BS18002245 BCA101 DP batch was tested for visual observations and shown to comply with each of the specification up to 24 months from the date of manufacture (see Table 17). The BL.14.0901/R/17/021 F DP BCA101 DP batch was not tested for visual observations.

### pH Range

Based on the optimal stability for BCA101 DP, the pH range of BCA101 DP was set to a pH 6.00±0.30 units. Any change in pH is an indication of degradation of one or more of the components in the formulation. As shown in FIG. 40, and Tables 16 and 17, both BCA101 DP batches complied with the pH specification up to 24 months from the date of filling/manufacture. From the trend analysis for DS batches, there was no appreciable change in pH detected (FIG. 40, Table 16, and Table 17).

### Osmolality

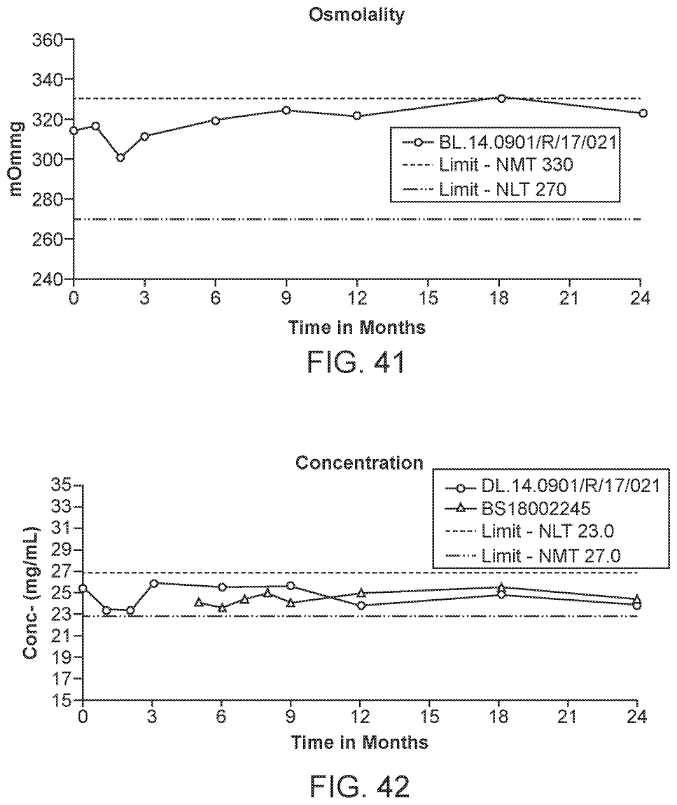

Osmolality is one of the parameters, which needs to be controlled for parenteral formulation close to that of blood plasma in order to avoid adverse reactions associated with injecting hypo/hypertonic DP during administration. The iso-osmotic concentration for BCA101 DP was majorly achieved using sucrose and the osmolality specification was set to 270 to 330 mOsmol/kg. Any change in sucrose concentration during the stability period can lead to a change in osmolality indicating an impact on the stability of the protein. As shown in FIG. 41, and Table 16, the osmolality of the BL.14.0901/R/17/021 F DP BCA101 DP batch was within the specification for up to 24 months from the date of file/manufacture. The BS18002245 BCA101 DP batch was not tested for osmolality.

### Protein Concentration

Protein concentration provides information about the quantity of the BCA101 DP in the sample. Limits for protein concentration were set at 25.00±2.00 mg/mL based on the developmental data. As shown in FIG. 42 (and Tables 16 and 17), both of the BCA101 DS batches complied with the specification up to 24 months from the date of filling/manufacturing.

### Visible Particles

The specification for visible particles was set as: “Injectable preparations should be practically free from visible particles” based on USP <790> visible particulates in injections and USP <1> injections (constituted solutions). The BCA101 DP vials were manufactured in a controlled environment and were inspected for visible particulates before the batch release. No visible particles were observed at the time of the batch release and over the course of the stability study (24 months) (which is not expected to change as the vials were aseptically sealed). The BS18002245 BCA101 DP batch complied with the specification up to 24 months from the date of manufacture (see Table 17). The BL.14.0901/R/17/021 F DP BCA101 DP batch was not tested for visible particles.

### Sub-Visible Particulate Matter

There were two specifications defined based on the sub-visible particle size based on pharmacopoeia limits (USP<788>). For sub-visible particles (>/=10 μm): not more than (NMT) 6000 particles per container; for sub-visible particles (4=25 μm): not more than (NMT) 600 particles per container. The BS18002245 BCA101 DP batch complied with the specification up to 24 months from the date of manufacture (see Table 17). The BL.14.0901/R/17/021 F DP BCA101 DP batch was not tested for sub-visible particulate matter.

### Sterility Test and BET (Bacterial Endotoxin Test)

The acceptance criterium for BET and sterility was based on the standard pharmacopoeia limits, which are specified as less than 0.25 EU/mg and “absence of microbial growth”, respectively. The BS18002245 BCA101 DP batch stored at 5±3° C. for 24 months from the date of manufacture complied with the acceptance criterium for both BET and sterility (see Table 17). The BL.14.0901/R/17/021 F DP BCA101 DP batch was not tested for BET and sterility.

### Extractable Volume

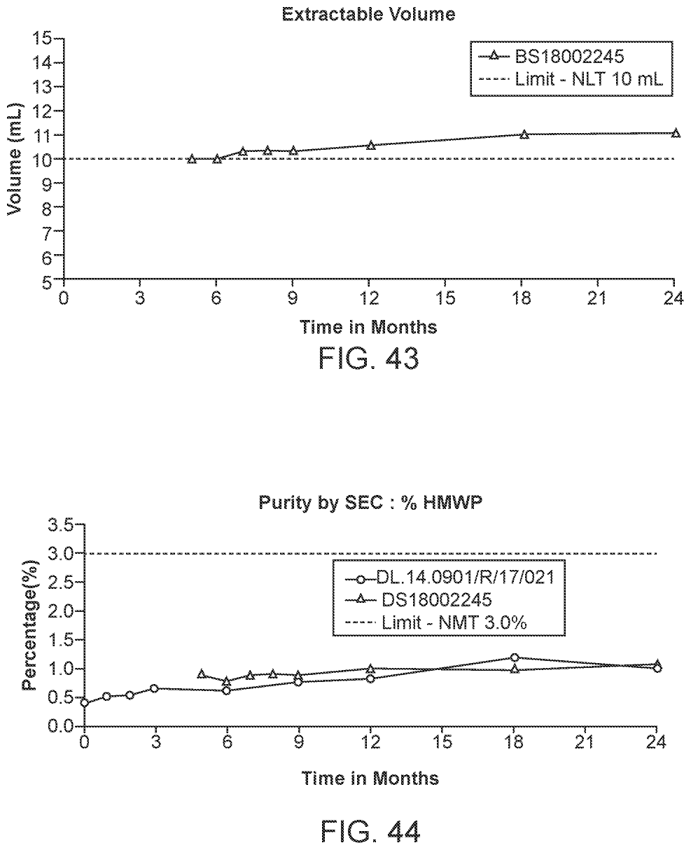

Complete 100% withdrawal of the product from the vial is not possible due to dead volume present in the vials and stoppers, which are in contact with the product during storage. It was determined to be ˜0.15-0.2 ml and on consideration of filling accuracy of the machine; and the fill volume for BCA101 DP was set at not less than (NLT) 10.0 mL. The extractable volume was therefore set at NLT 10.0 ml. As shown in FIG. 43 (and Table 17), the BS18002245 BCA101 DP batch complied with the specification limits at 24 months from the date of manufacture. The BL.14.0901/R/17/021 F DP BCA101 DP batch was not tested for extractable volume.

### Seal Integrity Test

The container closure seal integrity test (CCIT) complements the sterility test and is performed to ensure microbiological integrity (sterility) during storage and shipment up to the end of the DP shelf life. The acceptance criteria for CCIT was set to “none of the vials tested should contain any trace of coloured solution.” The BS18002245 BCA101 DP batch complied with the specification at 24 months from the date of manufacture (see Table 17). The BL.14.0901/R/17/021 F DP BCA101 DP batch was not tested by CCIT.

### Purity by SEC-HPLC

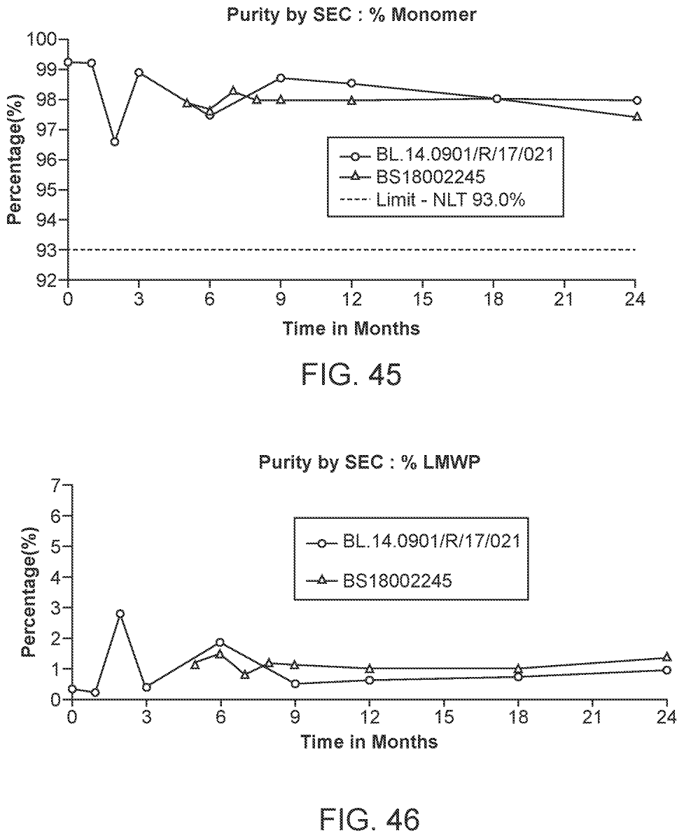

SEC-HPLC provides information about product monomer content and related HMWPs. The acceptance criteria for BCA101 DS were set as follows: % Monomer NLT 93.00%, % HMWP NMT 3.00%, and LMWP result to be reported. As shown in FIG. 44 (% HMWP), FIG. 45 (% monomer), and FIG. 46 (% LMWP), the SEC-HPLC purity results complied with the specification limits for both BCA101 DP batches until 24 months from the date of filling/manufacture.

### Purity by RP-HPLC

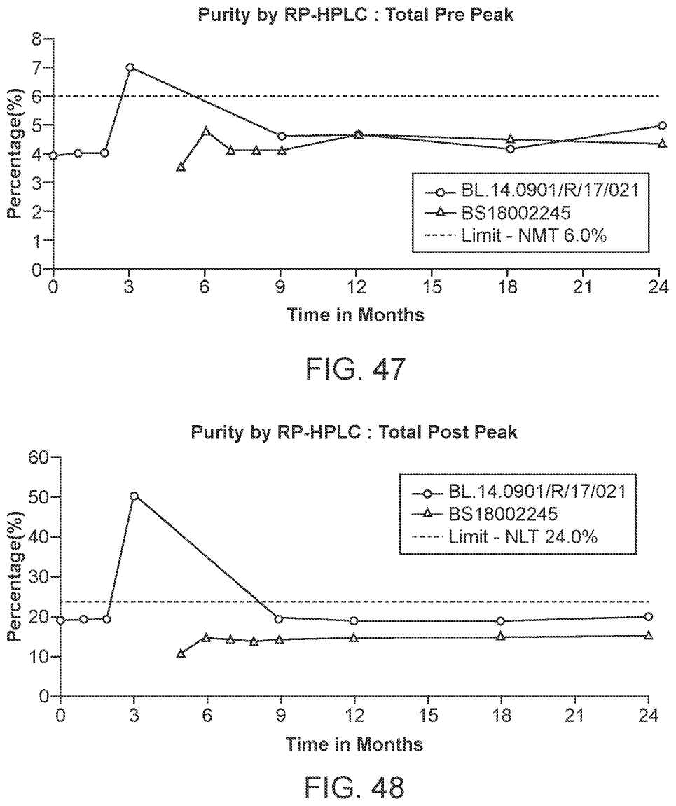

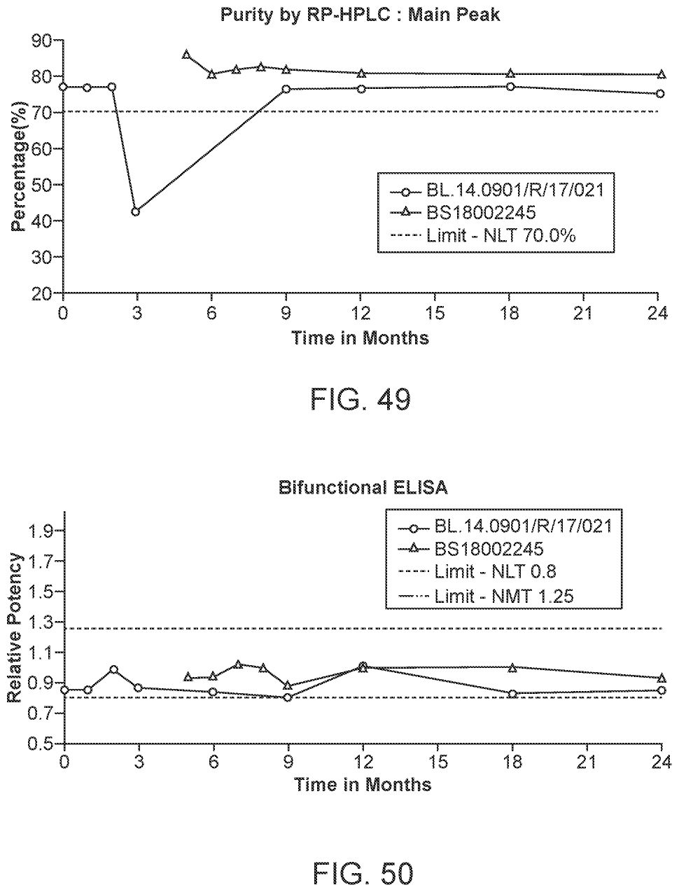

RP-HPLC provides information about the product purity with respect to hydrophobic variants. These acceptance criteria for BCA101 DS were set as follows: Main Peak: NLT 70.0%, Total Post Peaks: NMT 24%, and Total Pre-Peaks: NMT 6%. As shown in FIG. 47 (total pre peak), FIG. 48 (total post peak), and FIG. 49 (% main peak), the RP-HPLC purity results complied with the specification limits for the BS18002245 BCA101 DS batch tested until 24 months from the date of manufacture.

Excluding the T3M time point for the BL.14.0901/R/17/021 F DP BCA101 DP batch, the product related hydrophobic variants of the DP batches complied with the specification limits at 24 months for both BCA101 DP batches from the date of manufacture. The out-of-specification results observed at T3M alone for the BL.14.0901/R/17/021 F DP BCA101 DP batch was due to analytical assay deviation; since it was observed that the stability trend for subsequent time-points up to 24 months complies with the acceptance criteria. This behavior was not observed with other analytical tests and the BS18002245 BCA101 DP batch stability trend up to 24 months from date of manufacturing which was within specified limits.

### Bi-Functional ELISA

A bi-functional ELISA was used to determine the binding efficacy of BCA101 to EGFR and TGFβ1 ligand simultaneously. Briefly, recombinant hEGFR Fc coated plates were blocked and subsequently incubated with BCA101 for about 1 hour, followed by incubation with recombinant hTGFβ1. hTGFβ1 bound to hTGFβRII ECD moiety of BCA101 was then detected with biotinylated anti-hTGFβ1 antibody followed by streptavidin-HRP. Thereby, the signal will be obtained only when both binding arms are intact. The assay acceptance criterium was set to average relative potency of 0.80 to 1.25 with respect to reference standard.

As shown in FIG. 50 (and Table 16), the relative potency of the BL.14.0901/R/17/021 F DP BCA101 DP batch was well within the assay acceptance criterium until the last time-point tested, 24 months from date of filling/manufacture. Similarly, the BS18002245 BCA101 DP batch result was well within the assay acceptance criterium until the last time-point tested, 24 months from the date of filling/manufacture (see FIG. 50 and Table 17).

### Inhibition of Proliferation (10P) Assay

An inhibition of proliferation (TOP) assay was used to determine the ability of BCA101 DP inhibit FaDu cancer cell growth by binding to its target EGFR. This assay provides a method to determine the number of viable cells in culture by quantitating the amount of ATP present. The read out was based on luminescence by mono-oxygenation of luciferin which is catalyzed by luciferase in the presence of Mg2+, and ATP released by viable cells. The assay acceptance criterium was set to an average relative potency of 0.80 to 1.25 with respect to reference standard.

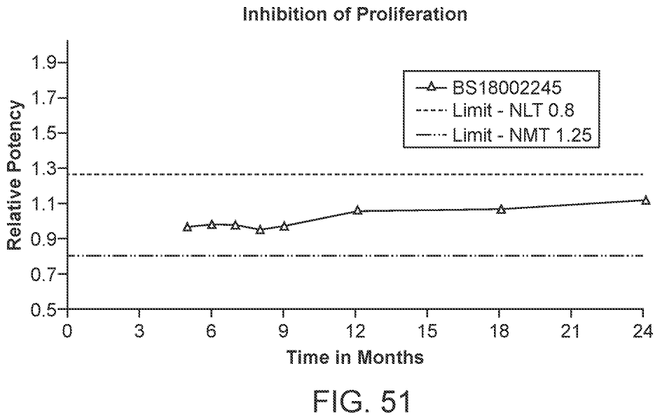

As shown in FIG. 51 (and Table 17), the BS18002245 batch BCA101 DP complied with the TOP specification up to 24 months from the date of filling/manufacture. The BL.14.0901/R/17/021 F DP BCA101 DP was not tested by TOP.

### Conclusions

The above data shows that multiple BCA101 drug product batches stored at 5±3° C. met the acceptance criteria up to 24 months from the date of filling/manufacture. Based on the stability trend analysis, no considerable change in pH, osmolality, sub-visible particle number per container, SEC-HPLC, Protein Content, RP-HPLC, or functionality were observed. The data obtained from the R&D (BL.14.0901/R/17/021 F DP) and developmental GMP batch (BS18002245) indicates physico-chemical and functional stability of the BCA101 DP over 24 months from the date of filling/manufacturing when stored at 5±3° C. in USP Type 1 glass vials. The BS18002245 BCA101 DP batch further complied with the specification limit for BET and sterility for longest time point tested, 24 months from the data of filling/manufacture.

### Example 4. Physioco-Chemical and Biological Characterization of BCA101 DS

The objective of this study was to further characterize and confirm the physico-chemical and biological properties of two batches of BCA101 DS: 1) BL.14.0901/R/17/021/F DS a pre-clinical R&D batch and internal reference standard comprising 25.58 mg/ml BCA101, 0.02% w/v polysorbate 20, 8.0% w/v sucrose, and 10 mM citrate phosphate (citric acid monohydrate 0.573 mg/mL, disodium Hydrogen phosphate dihydrate 1.294 mg/mL) buffer (pH 6.0); and 2) GF19000040 a GMP batch comprising 26.60 mg/ml BCA101, 0.02% w/v polysorbate 20, 8.0% w/v sucrose, and 10 mM citrate phosphate (citric acid monohydrate 0.573 mg/mL, disodium Hydrogen phosphate dihydrate 1.294 mg/mL) buffer (pH 6.0). Table 18 provides a summary of the analytical tools employed for the evaluation of BCA101 physico-chemical and biological quality attributes in the present example.

TABLE 18
Summary of methodologies employed for
characterization of BCA101 DS batches

| | Test/Technique/Method of |
|---|---|
| Molecular Parameter/Attributes | Characterization |
| Product related size-variants | Size-Exclusion HPLC |
| | Non-Reduced Capillary Electrophoresis |
| | Sodium Dodecyl Sulphate |
| | Reduced Capillary Electrophoresis |
| | Sodium Dodecyl Sulphate |
| Product related charge variants | Imaged Capillary Electrophoresis (iCE) |
| Primary Structure | Intact Mass Analysis |
| | Reduced Peptide Mass Fingerprinting |
| | with High Resolution MS and MS² |
| Higher order structure | Circular Dichroism |
| (Secondary and Tertiary | Disulphide Linked Peptide Analysis |
| Structure) | |
| Glycosylation | 2-AA labelled N- Glycan Analysis |
| | Sialic acid estimation |
| Biological Characterization | Bi-functional ELISA |
| | Inhibition of Proliferation (IOP) |
| | Antibody Dependent Cellular |
| | Cytotoxicity (ADCC) |
| | TGFβ SMAD Assay |
| Product Related Hydrophobic | Reversed-Phase HPLC |
| Variants | |

The data in the present example shows the comparability of the BCA101 GMP DS batch GF19000040 with the internal reference standard batch BL.14.0901/R/17/021/F DS; and shows the compliance of both batches with the set quality attribute specifications. A summary of the physico-chemical and biological characterization conducted in the present example is provided below and in Table 19, with an additional detailed description of each of the analyses performed provided below.

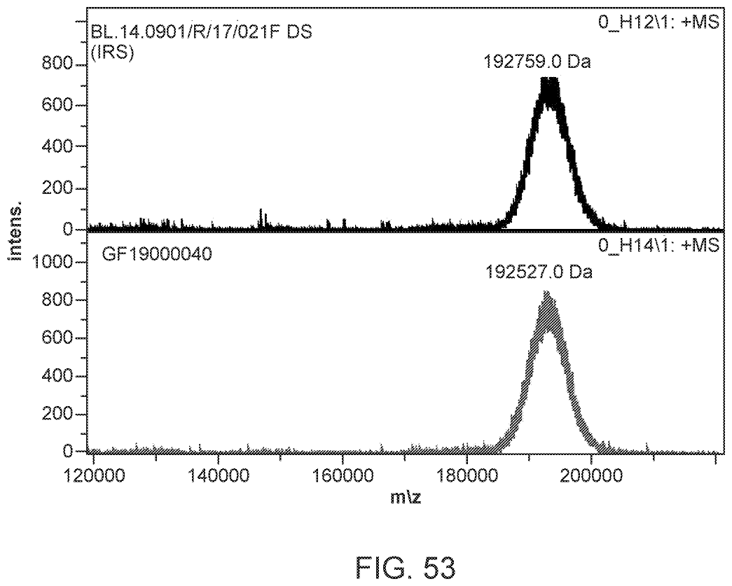

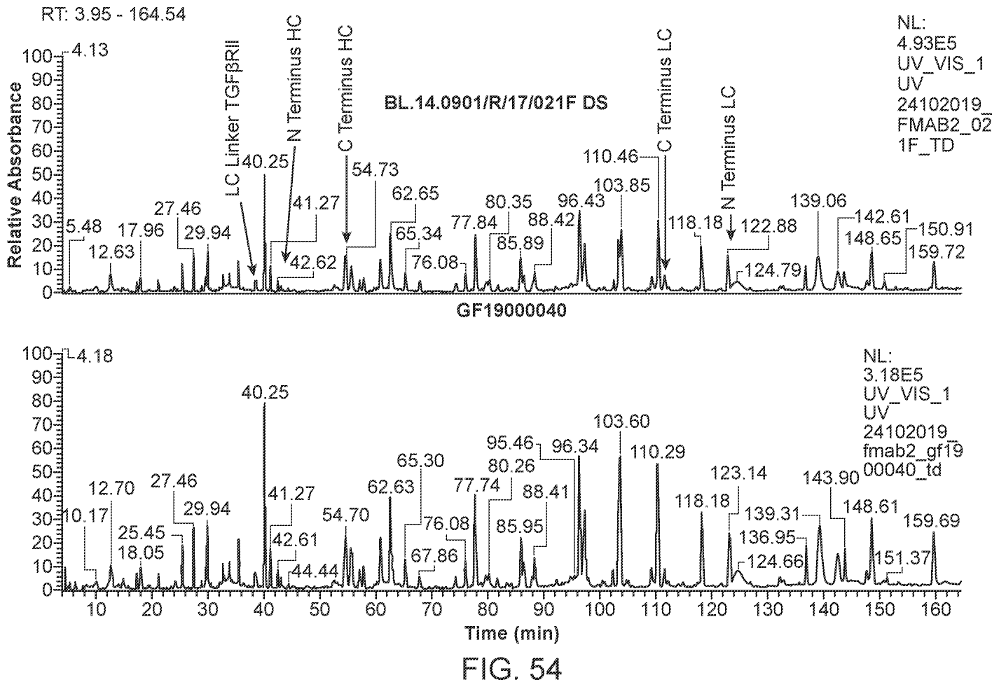

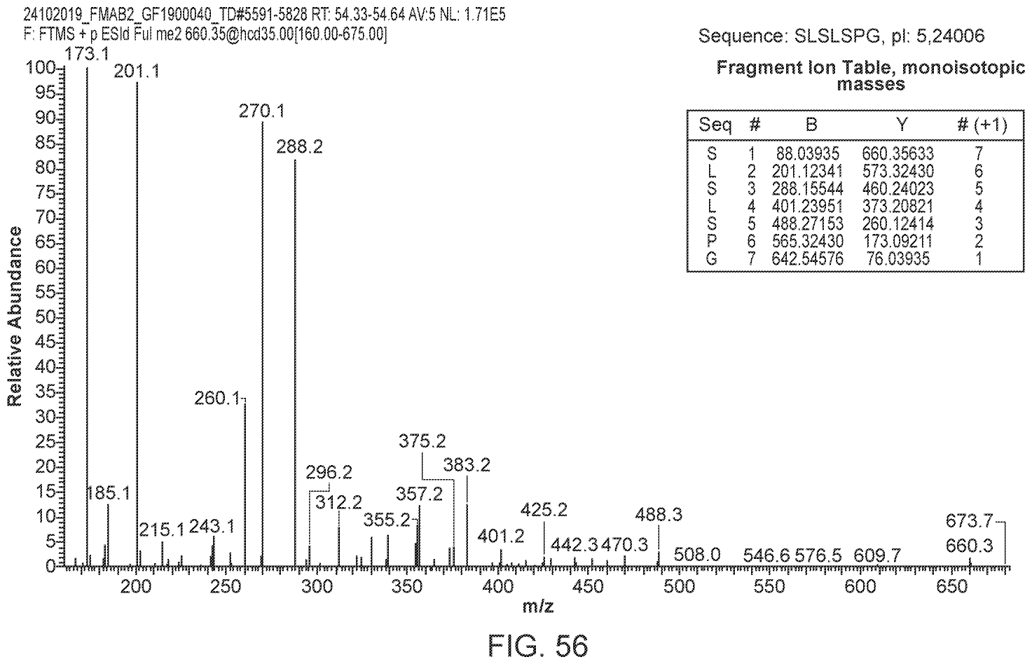

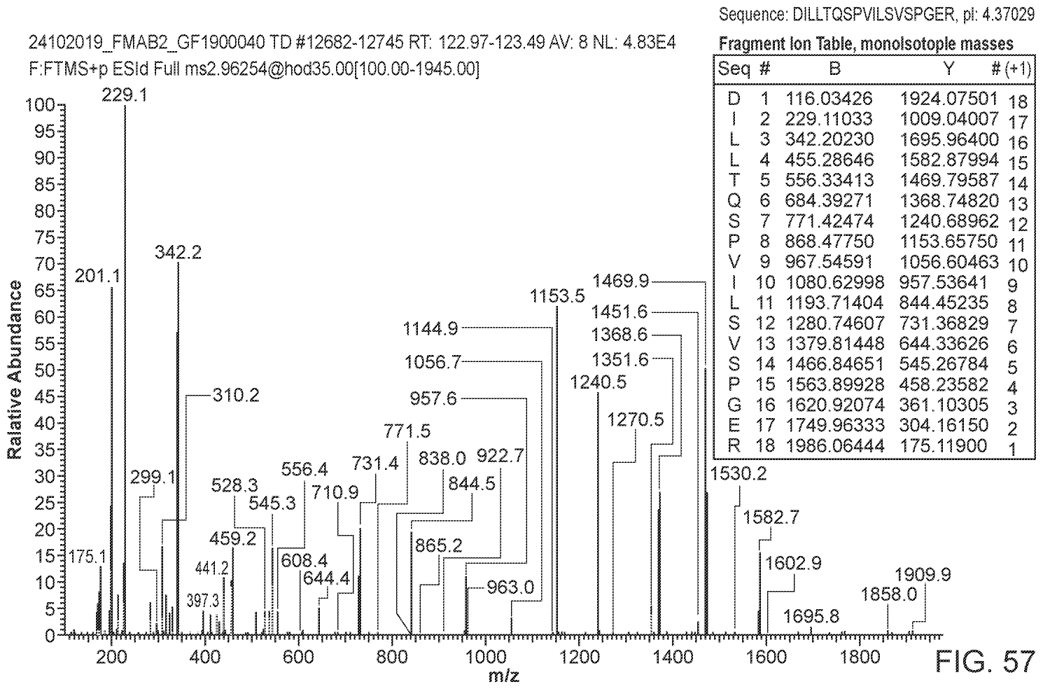

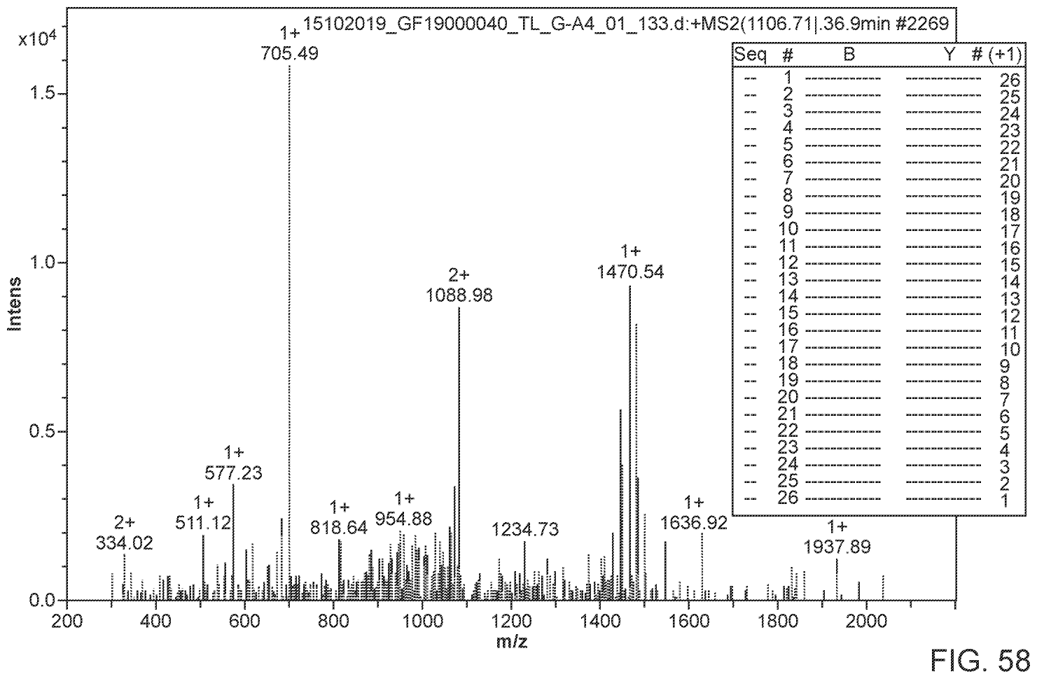

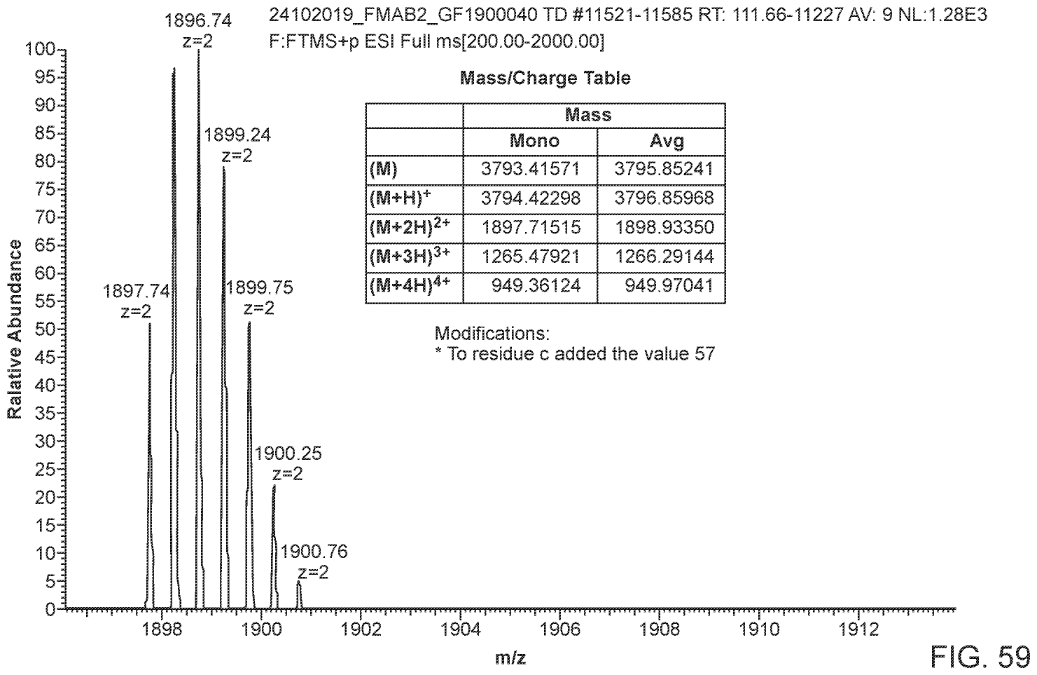

Primary Structure: The intact molecular mass of the GF19000040 batch was found to be comparable to the BL.14.0901/R/17/021/F DS batch and both within the quality attribute specification (FIG. 53 and Table 23). The intact molecular mass was found to be higher than the expected theoretical molecular mass due to extensive glycosylation. The peptide sequence of the GF19000040 batch and the BL.14.0901/R/17/021/F DS batch, including N- and C-terminal and linker sequence, was consistent and confirmed using multiple enzyme PMF method (FIGS. 54-59).

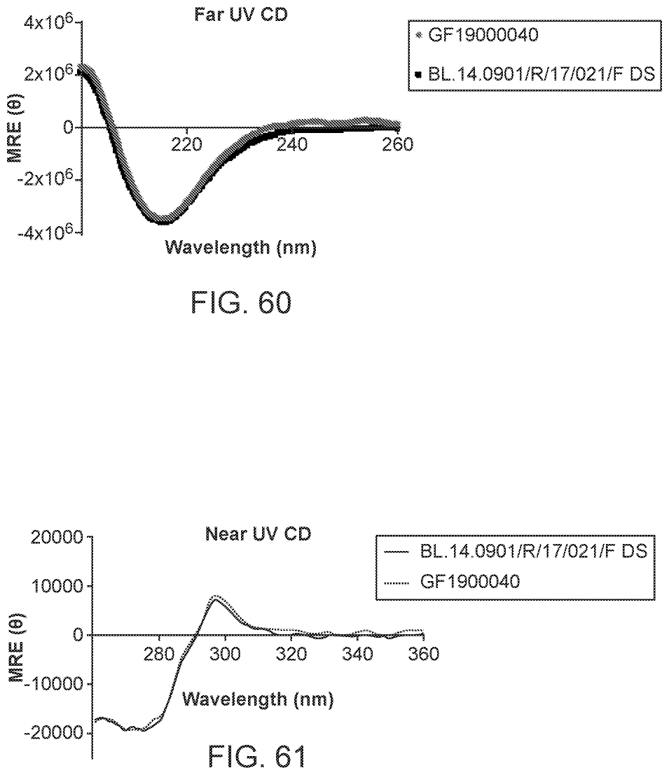

Secondary and higher order structure: The far and near-UV CD profiles of the GF19000040 batch were found to be comparable to that of the BL.14.0901/R/17/021/F DS batch, and both within the quality attribute specification (FIGS. 60-61). A negative signature peak was observed around 215 nm indicating a characteristic β-sheet protein. All the disulfide linkages present in the GF19000040 batch were identified and confirmed to align with the BL.14.0901/R/17/021/F DS batch (Tables 24-26).

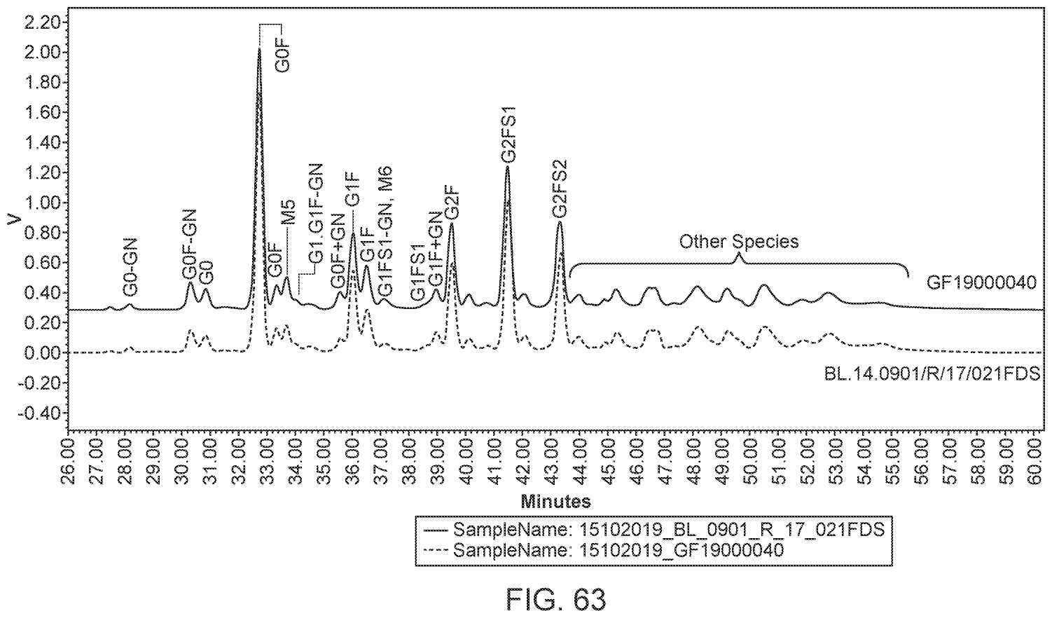

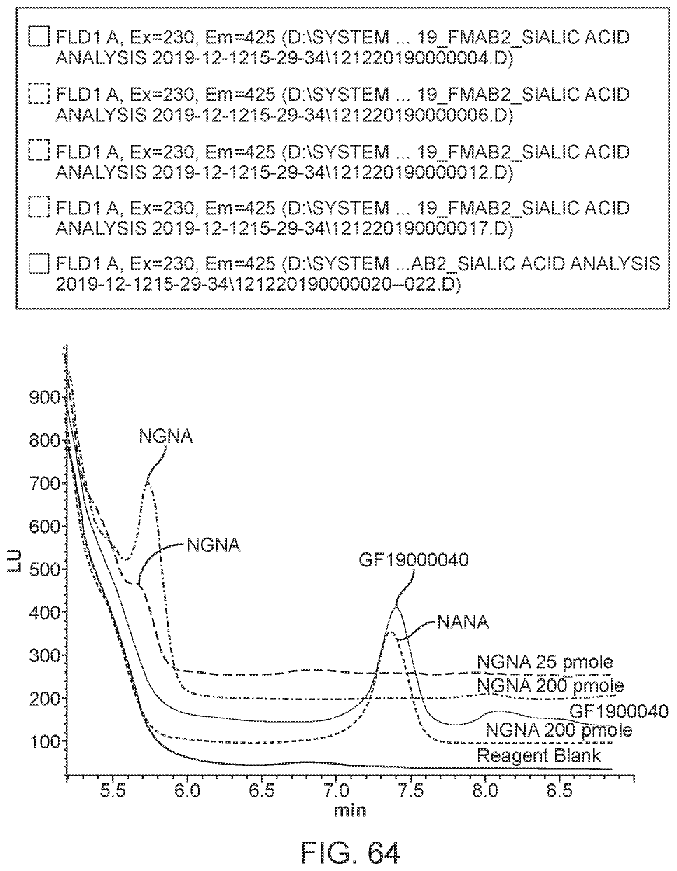

Glycosylation: The N-glycan profile for the GF19000040 batch was determined to be comparable to that of the BL.14.0901/R/17/021/F DS batch, and within the quality attribute specification (Tables 27-28, and FIGS. 63-64). The relative abundance of the major glycoforms in the GF19000040 batch were determined to be comparable with those in the BL.14.0901/R/17/021/F DS batch. Sialic acid content in the GF19000040 batch was estimated to be 8.8 moles/mole of protein; and that of the BL.14.0901/R/17/021/F DS batch determined to be 12.2 moles/mole of protein. This difference in average sialic acid content was determined to be within the method variability and to have little influence on the biological function.

Biological Activity: The biological activity of the BCA101 batches was evaluated for its ability to simultaneously bind its targets (EGFR and TGFβ1), inhibit signaling through the cognate receptors, and trigger ADCC through the Fc domain. The overall data from the biological assays showed that the biological activity of both GF19000040 and BL.14.0901/R/17/021/F DS batches is comparable, and within quality attribute specifications (Tables 30-32).

Product related variants: Size Variants—The percent monomer, HMWP and LMWP species were comparable in both the GF19000040 and BL.14.0901/R/17/021/F DS batches as analyzed by SEC-HPLC, and within the quality attribute specifications (Table 20). The relative percent of fragments quantitated using nrCE-SDS and rCE-SDS were also determined to be comparable between the GF19000040 and BL.14.0901/R/17/021/F DS batches and within the specification (Table 21). Charge Variants—The charge variants as obtained post integration of iCE analysis were termed as Region 1, Region 2, and Region 3. The profiles corresponded to each other and the values for the GF19000040 and BL.14.0901/R/17/021/F DS batches were determined to be comparable and within the quality attribute specifications (Table 22). Hydrophobic variants—The RP profiles showing main, total pre-peaks, and total post-peaks and the corresponding relative area percent were determined to be comparable between the GF19000040 and BL.14.0901/R/17/021/F DS batches, and within the quality attribute specifications (Table 29).

TABLE 19
Summary of physico-chemical characterization tests performed for BCA101 DS batches

| Molecular | | |
|---|---|---|
| Parameter | Test | Results |
| Protein | UV 280 | 26.00 ± 2.0 mg/mL |
| Concentration | | |
| Product related | SEC-HPLC | The monomer content in both the batches were observed to be |
| size-variants | | 99.4 and 99.3 for BCA101 DS GF19000040 and BCA101 DS |
| | | BL.14.0901/R/17/021/F, respectively, which is within the |
| | | specification of NLT 90%. The relative proportion of HMWP |
| | | and LMWP are comparable for both the batches. |
| | nrCE-SDS | The nrCE-SDS analysis for BCA101 DS |
| | | BL.14.0901/R/17/021/F and BCA101 DS GF19000040 |
| | | displayed similar corrected area percentage for main peak group |
| | | 94.4 and 94.3, respectively. |
| | rCE-SDS | The rCE-SDS analysis for the BCA101 DS |
| | | BL.14.0901/R/17/021/F and BCA101 DS GF19000040 batches |
| | | displayed similar corrected area percentage of sum of peaks 1, 2 |
| | | and 3 as 97.8 and 97.5, respectively. |
| Product related | iCE | The iCE analysis displayed similar area percentage for both |
| charge variants | | BCA101 DS BL.14.0901/R/17/021/F and BCA101 DS |
| | | GF19000040 in terms of R1 (44.6 and 50.4 and4 4.6), R2 (16.0 |
| | | and 16.4) and R3 (33.6 and 39.1). |
| Primary | Intact Mass | The theoretical molecular mass of BCA101 based on amino acid |
| Structure | Analysis | sequence is 178105 Da. The observed molecular masses for |
| | | BCA101 DS GF19000040 and BCA101 DS |
| | | BL.14.0901/R/17/021/F were observed to be ~192 kDa. The |
| | | intact mass spectral dispersion was comparable for both the |
| | | batches and ranges from 180 kDa-210 kDa. |
| | Reduced | The UV chromatogram for both BCA101 DS GF19000040 and |
| | Tryptic | BCA101 DS BL.14.0901/R/17/021/F corresponded to each |
| | Peptide mass | other and the sequences were observed to be identical. |
| | fingerprinting | |
| | with high | |
| | resolution | |
| | MS and MS² | |
| Higher order | Secondary | The profiles corresponded with each other indicating similar |
| structure | and tertiary | secondary and tertiary structure. The negative signature peak in |
| (Secondary | structure | Far UV spectra observed around 215 nm indicated a |
| and Tertiary | | characteristic β-sheet structure in both the batches. The |
| Structure) | | secondary structure for BCA101 DS GF19000040 and BCA101 |
| | | DS BL.14.0901/R/17/021/F corresponded to each other. |
| | Disulphide | All expected peptides with disulphide bonds were present in both |
| | bridges | batches. BCA101 DS GF19000040 and BCA101 DS |
| | | BL.14.0901/R/17/021/F were observed to be similar in mass. |
| Glycosylation | N- Glycan | The fluorescence NP-HPLC glycan profiles of BCA101 DS |
| | Analysis | GF19000040 and BCA101 DS BL.14.0901/R/17/021/F were |
| | | highly similar and the relative glycan distribution of glycoforms |
| | | was comparable. |
| | Sialic acid | Sialic acid content in BCA101 DS GF19000040 was estimated |
| | estimation | to be 8.8 moles/mole of protein and that of BCA101 DS |
| | | BL.14.0901/R/17/021/F was 12.2 moles/mole of protein. This |
| | | difference in average sialic acid content is within the method |
| | | variability and has little influence on the biological function. |
| Biological | Bi-functional | The BCA101 DS GF19000040 displayed a relative potency |
| Characterization | ELISA | value of 0.93 with respect to BCA101 DS |
| | | BL.14.0901/R/17/021/F, which is well within the acceptance |
| | | criteria of 0.80-1.25 and also displayed the % CV of ≤20 |
| | | indicating similar potency for bi-functional activity. |
| | Inhibition of | The BCA101 DS GF19000040 displayed relative potency value |
| | proliferation | of 0.99 with respect to BCA101 DS BL.14.0901/R/17/021/F, |
| | (IOP) | which fall within the assay acceptance criteria of 0.80-1.25 |
| | | indicating similar potency for inhibition of FaDu cell |
| | | proliferation. |
| | TGFβ SMAD | The BCA101 DS GF19000040 was evaluated for TFGβ SMAD |
| | Assay | Assay and displayed a relative potency value of 1.10 with respect |
| | | to BCA101 DS BL.14.0901/R/17/021/F, which is well within the |
| | | accepted range of 0.80 to 1.25 and also displayed the % CV |
| | | of ≤20 indicating similarity to BCA101 DS |
| | | BL.14.0901/R/17/021/F. |
| | Cytokine | BCA101 DS GF19000040 did not show secretion for any of the |
| | release assay | tested cytokines in the assay; even at high concentration of |
| | (CRA) | 1 mg/mL. This indicates that the tested BCA101 batches do not |
| | | induce immune cells to secrete cytokines (IL-1β, TNF, IFNγ, IL- |
| | | 6) non-specifically. |
| Cell based | ADCC | The BCA101 DS GF19000040 batch displayed the relative |
| Bioassays | | potency values of 1.23 with respect to BCA101 DS |
| | | BL.14.0901/R/17/021/F and it is well within the assay |
| | | acceptance criteria of 0.80-1.25 and ≤20% CV indicating similar |
| | | potency for ADCC activity. |
| Product Related | RP-HPLC | The RP-HPLC analysis provided the quantitation of hydrophobic |
| Hydrophobic | | variants (Main, total pre-peaks and post-peaks) in BCA101. The |
| variants | | total main peak content in both the batches were observed to be |
| | | 82.2 and 81.3 for both for BCA101 DS GF19000040 and |
| | | BCA101 DS BL.14.0901/R/17/021/F and matched the |
| | | specification of NLT 70%. The Total Pre peak content for both |
| | | BCA101 DS GF19000040 and BCA101 DS |
| | | BL.14.0901/R/17/021/F was observed to be 4.9 and 4.8 and |
| | | within the specification of NMT 6.0%. Total post- peaks content |
| | | for both BCA101 DS GF19000040 and BCA101 DS |
| | | BL.14.0901/R/17/021/F were observed to be 12.9 and 13.9, |
| | | respectively and matches the specification not more than 24.0%. |
| | | Therefore, the two batches were observed to be similar in |
| | | proportion of hydrophobic variants. |

### Purity of BCA101 DS by SEC-HPLC

Product related size variant impurities include high molecular weight protein (HMWP) species, low molecular weight protein (LMWP) species and fragments. The HMWP species are formed due to association of two or more molecules of the monomer. The primary method of analysis for HMWP separation and estimation is size exclusion chromatography HPLC (SEC-HPLC), which was employed here. As shown in Table 20, The SEC-HPLC profiles of the first batch of BCA101 DS (GF19000040) and the second batch BL.14.0901/R/17/021/F DS (internal reference standard) were visually similar (chromatograms not shown) and the relative percentage of Monomer, HMWP and LMWP were determined to be comparable (see Table 20). Additionally, levels of Monomer, HMWP and LMWP for both the BCA101 DS batches are within the specifications of NMT 3.0% for HMWP and 7.0% for LMWP, respectively (see Table 20).

TABLE 20
Purity of BCA101 DS analyzed by SEC-HPLC

| BCA101 DS Batch | % HMWP | % Monomer | % LMWP |
|---|---|---|---|
| GF19000040 | 0.50 | 99.4 | 0.1 |
| BL.14.0901/R/17/021/FDS | 0.50 | 99.3 | 0.2 |

Purity of BCA101 DS by nrCE-SDS and rCE-SDS

Capillary electrophoresis (CE) is an automated and instrumental version of traditional slab gel electrophoresis (SDS-PAGE) that employs narrow-bore (20-200 μm i.d.) capillaries to perform high efficiency separations of both large and small molecules. These separations are facilitated by the use of high voltages, which may generate electro-osmotic and electrophoretic flow of buffer solutions and ionic species, respectively, within the capillary. CE-SDS (reduced and non-reduced) is used for the quantitative analysis of purity and size-based heterogeneity of therapeutic products. Both BCA101 DS batches, GF19000040 and BL.14.0901/R/17/021/F DS, were analyzed using non-reduced and reduced CE-SDS to quantify the product related fragments. The (non-reduced (nr) and reduced (r)) CE-SDS electopherograms show similarity across the two batches (chromatograms not shown), and both within the quality attribute specifications (Table 21). The main peak group in nrCE-SDS was observed to be broad and bifurcated which could arise from heterogeneous conformations of the denatured state due to a) incomplete denaturation of a large protein and b) heterogeneity in glycoforms. The nrCE-SDS analysis of the GF19000040 batch and BL.14.0901/R/17/021/F DS batch displayed corrected area percentage of main peak group as 94.3% and 94.4% respectively. The rCE-SDS analysis displayed that the proportion of the sum of the most abundant species (peak1+peak2+peak3) in GF19000040 batch and IRS is 97.5% and 97.8% respectively. Therefore, the proportion of fragments are similar across the two batches tested, and within the specifications (Table 21).

TABLE 21
Purity of BCA101 DS analyzed by nrCE-SDS and rCE-SDS

| | nrCE-SDS | | | rCE-SDS | | |
|---|---|---|---|---|---|---|
| | % | % Main | % Other | % | % Sum of Peak | % Post |
| BCA101 DS Batch | Fragments | Peak Group | Peaks | Fragments | 1, 2, and 3 | Peaks |
| GF19000040 | 5.7 | 94.3 | 0.0 | 0.6 | 97.5 | 1.9 |
| BL.14.0901/R/17/021/FDS | 5.6 | 94.4 | 0.0 | 1.2 | 97.8 | 1.1 |

### Purity of BCA101 DS by iCE

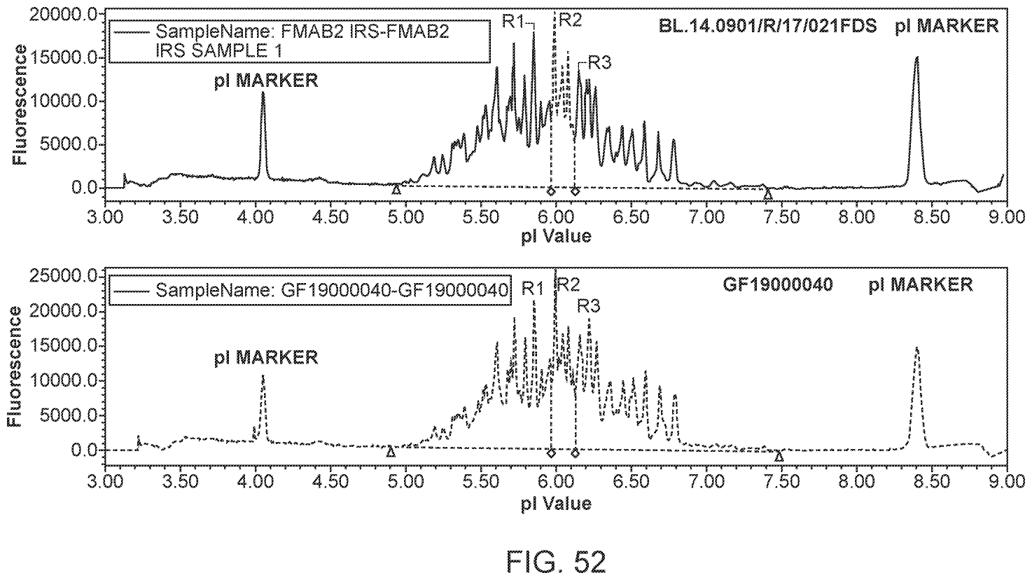

Imaged capillary electrophoresis (iCE) is an analytical tool widely employed for separation and quantitation of product related charge variants in biotherapeutics. This technique uses the principal of protein charge separation in a pH gradient gel matrix under an applied current. Based on the net charge, proteins migrate in the gel matrix until an equilibrium of pH unit with the molecular isoelectric point (pI) is achieved. The charge variants profile of both batches of BCA101 DS were assessed using iCE technique. Due to heterogeneity of BCA101 arising from heavy sialylation, the charge variant profile shows multiple peaks with lack of baseline separation. Therefore, for ease of comparison the peaks were clustered into regions R1, R2 and R3 as shown in FIG. 52. Relative proportion of regions R1, R2 and R3 were estimated from relative area under the curve for each cluster. As shown in Table 22, the two batches were observed to be comparable within method variation and within the specifications.

TABLE 22
Purity of BCA101 DS analyzed by iCE

| BCA101 DS Batch | R1% | R2% | R3% |
|---|---|---|---|
| GF19000040 | 44.6 | 16.4 | 39.1 |
| BL.14.0901/R/17/021/F DS | 50.4 | 16.0 | 33.6 |

### Intact Mass Analysis

The intact mass analysis of BCA101 was performed using matrix assisted laser desorption and ionization-time of flight mass spectrometer (MALDI-TOF-MS). MALDI-TOF-MS is a routine and rapid qualitative tool performed by application of a beam of laser into the analyte embedded in rapidly ionizing matrix. The matrix absorbs the ultraviolet light from the laser (nitrogen laser of wavelength 337 nm) and converts it to heat energy. A small part of the matrix heats rapidly and is vaporized, together with the sample and the resultant spectra from the ionization produces singly charged ions. Based on the cluster size distribution of the mass spectra, the software demarcates the average molecular mass of the sample analyzed. The intact mass spectrum of the GF19000040 batch and the BL.14.0901/R/17/021/F DS is shown in FIG. 53 and Table 23.

As described above, the theoretical molecular mass of BCA101 based on amino acid sequence is 178105 Da. As shown in Table 23, the observed molecular masses for BCA101 in the GF1900040 batch and the BL.14.0901/R/17/021/F DS batch were observed to be −192 kDa, which is higher than the theoretical mass of the molecule due to glycosylation. Due to heterogeneity of N-linked glycosylation in BCA101 the mass spectra show broad molecular weight distribution in the range 180 kDa to 210 kDa.

TABLE 23
Intact mass of BCA101 DS analyzed by MALDI-TOF-MS

| | Theoretical Molecular | Observed Molecular |
|---|---|---|
| BCA101 DS Batch | Weight (Da) | Mass (Da) |
| GF19000040 | ~178105 | 192759.0 |
| BL.14.0901/R/17/021/FDS | | 192527.0 |

### Peptide Mass Fingerprinting

Reduced peptide mass fingerprinting (PMF) provides detailed information of a protein, which includes determination of the primary sequence, N- and C-terminus sequence, identification of site and type of post-translational modifications, etc. This approach subjects the molecule to specific enzymatic digestion procedures followed by a chromatographic separation of peptides prior to MS and/or MS² analysis. A tandem MS or MS² approach facilitates the evaluation of the primary structure in terms of the linker confirmation and N- and C-terminal sequencing. The sequencing of N- and C-terminus is an important dataset to confirm the start and end of the protein sequence of interest. The PMF analysis has been widely utilized by the pharmaceutical industry to generate a unique fingerprint for the molecule of interest and to aid in the identification of the complete protein sequence coverage. The PMF profile overlay of BCA101 batch GF19000040 and BCA101 batch BL.14.0901/R/17/021/F DS was established (FIG. 54). For LC-TGFβRII 100% sequence coverage could be observed using Trypsin and Glu-C whereas for the HC, four enzymes, Trypsin, Glu-C, Asp-N, LysC, were used for 100% sequence coverage. The heavy chain and light chain-Linker-TGFβRII ECD fragments generated upon multiple enzyme digestion along with the theoretical and observed masses and their respective retention times were determined.

As shown in FIG. 54, the UV chromatograms of reduced-alkylated tryptic peptide mass fingerprinting of the GF19000040 batch along with the BL.14.0901/R/17/021/FDS batch are comparable to each other, and within specification. The MS 2 for the linker was found intact. No free end light chains were found using multiple enzymes (Glu-C, AspN, LysC) and therefore it could be concluded that the fusion is intact in both the batches.

### N- and C-Terminal Sequencing

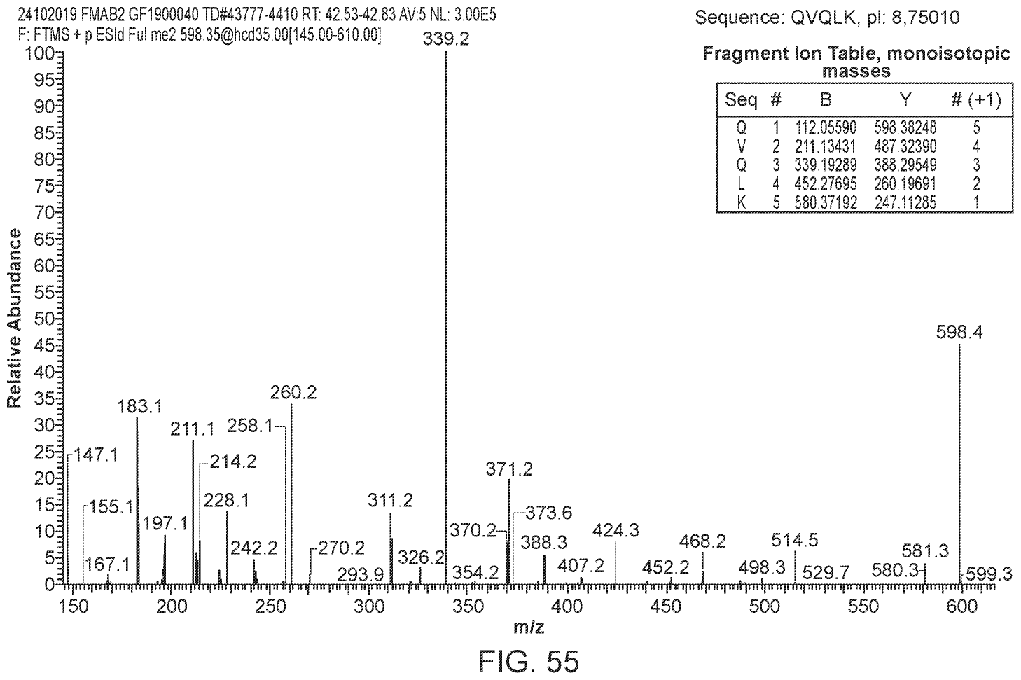

N- and C-terminal sequencing experiments were executed to confirm the start and end of the protein sequence of BCA101. PMF is a robust method, which provides the N- and C-terminal sequence using MS and MS 2 data and compares the experimental spectral data with in silico-generated masses of tryptic digested LC and HC fragments. The b and y daughter ion series of N- and C-terminal sequences of HC, LC and the linker as obtained from MS 2 spectra for the is presented in FIG. 55-FIG. 59.

From the data presented in FIG. 55-FIG. 59, the N- and C-terminal sequence of heavy chain and light chain-TGFβRII was confirmed as N-terminal HC: pyroQVQLK (SEQ ID NO: 35); C-terminal HC: SLSLSPG; N-terminal LC-TGFβRII: DILLTQSPVILSVSPGER (SEQ ID NO: 36); LC-Linker-TGFβRII: GECGGGGSGGGGSGGGGSTIPPHVQK (SEQ ID NO: 37). The C-terminus of TGFβRII ECD was not selected for MS 2 due to low intensity and high molecular weight, however the supporting charge states were visible (MS data provided above).

As shown in FIG. 54, the UV chromatogram of the tryptic peptide map of GF19000040 and BL.14.0901/R/17/021/F DS corresponded to each other, and the sequences were observed to be identical to the theoretical sequence. The first amino acid of the heavy chain at the N-terminus is “Gln” i.e “Q” which is observed as pyro Q in both the batches analyzed. Pyro-Glutamate (Q) is result of spontaneous cyclization of glutamine. The heavy chain C-terminal amino acid sequence ends with “PG” and does not show presence of lysine as “PGK”. The N terminus of light chain is intact and do not show any modification in FmAb2 batches. The C terminus of light chain is fused to the TGFβRII ECD. The C-terminal sequence of TGFβRII ECD was intact and determined to comply with the available theoretical sequence.

### Circular Dichroism (CD)

Circular dichroism is a form of light absorption spectroscopy that measures the difference in absorbance of right and left circularly polarised light by a substance. The secondary structure of a protein can be determined by CD spectroscopy in the “far-UV” spectral region (200-260 nm). At these wavelengths the chromophore is the peptide bond, and the signal rises when it is located in a regular, folded environment. α-helix, β-sheet and random coil structures each give rise to a characteristic shape and magnitude of CD spectrum.

The CD spectrum of a protein in the “near-UV” spectral region (260-350 nm) can be sensitive to certain aspects of tertiary structure. At these wavelengths the chromophores are the aromatic amino acids and disulfide bonds, and the CD signals they produce are sensitive to the overall tertiary structure of the protein. The signals in the region from 250-270 nm are attributable to phenylalanine residues, signals from 270-290 nm are attributable to tyrosine and those from 280-300 nm are attributable to tryptophan. Since the tertiary structure is protein specific, it does not have a standard profile.

FIG. 60 and FIG. 61 provide the far UV and near UV CD spectra of the BCA101 DS GF19000040 batch and the BCA101 BR.14.09015/R/16/021 DS batch, respectively. As shown in FIGS. 60-61, the profiles correspond with each other and the BCA101 BR.14.09015/R/16/021 DS batch displays wavelength minima at 216.2 nm and the BCA101 DS GF19000040 batch displays the minima at 215.4 nm, indicating similar secondary and tertiary structure. The negative signature peak in Far UV spectra for both the batches was observed to be around 215 nm indicating characteristic β-sheet structure.

### Non-Reduced Disulfide Bridging

The disulfide bond linkages between the heavy chain, light chain and heavy-light chains of the antibody backbone in BCA101 determines its structure, stability and biological function. The antibody backbone of BCA101 belongs to the IgG1 class, which has 16 disulfide bonds; 4 inter-chain disulfide bonds in the hinge region and 12 intra-chain bonds associated with different domains. Among the four inter-chain disulfides, two link together the heavy chains and the other two connects the light and heavy chains. The C-terminal end of the light chain Cys 214 forms a disulfide bond with Cys 222 in the heavy chain, which connects the light and heavy chains. Cys 228 and Cys 231 in each of the heavy chain link to form two parallel inter-chain disulfide bonds between the two heavy chains. The 4-polypeptide chains (2 heavy and 2 light chains) are connected by these 4 inter-chain disulfide bonds to form a tetramer, which plays a key role in the antibody backbone structure and function. Disulfide scrambling or incomplete formation of disulfide bonds may lead to loss of function. In addition, the TGFβRII—ECD domain of BCA101 is rich in Cys residues, which link to form 6 intra-chain disulfide bridges that plays a critical role in receptor binding domain structure and function. The disulfide linkages were analyzed to establish the presence of correct connectivity to ensure drug function and quality.

Mass spectrometry (ESI-MS) tools were employed to characterize disulfide bond structures, which involves cleavage of the protein by enzymatic means where the proteolytically derived peptides contain cysteine residues for determining the presence and locations of disulfide bonds. The peptide mixtures were directly analyzed by mass spectrometric peptide mapping. Molecular mass analyses were used to identify the disulfide-bonded dipeptides by assigning the observed ion signals to the corresponding calculated masses from the primary amino acid sequence. The disulfide bond structure of BCA101 was characterized using non-reduced peptide mapping. Generally, the method involves the characterization of disulfide-bonds without reduction through collision induced dissociation (CID) ensuring selective cleavage of the protein and generation of peptide mass fingerprinting under non-reduced conditions. The disulfide bond linkages observed in the antibody backbone and TGFβRII-ECD domains of BCA101 are presented in Table 24.

TABLE 24
Cysteine linkages in BCA101 along with their region and significance.

| Location | Cysteine Linkages | Regions | Significance |
|---|---|---|---|
| Heavy chain | Cys22-Cys95 | CDR region | The Cys22-Cys95 disulfide bond linkage |
| (Fab Domain) | | | is crucial for correct folding and are |
| | | | required for antigen binding activity |
| | Cys146-Cys202 | Constant domain | Intra chain disulfide linkage confirms |
| | | (CH1) | integrity of constant domain |
| Heavy chain | H-Cys228-H- | Hinge region | Hinge region disulfide linkages are |
| (Hinge) | Cys228 | | critical for the bond between two heavy |
| | H-Cys231-H- | | chains to maintain antibody structure |
| | Cys231 | | |
| Heavy chain | Cys263-Cys323 | Constant domain | Affects the stability and monomer-dimer |
| (Fc Domain) | | (CH2) | equilibrium of the human CH2 domain |
| | Cys369-Cys427 | Constant domain | Affects the stability and monomer-dimer |
| | | (CH3) | equilibrium of the human CH3 domain |
| Light chain | Cys23-Cys88 | CDR region | The Cys23-Cys88 disulfide linkage is |
| (Fab Domain) | | | crucial for correct folding and are |
| | | | required for antigen binding activity |
| | Cys134-Cys194 | Constant domain | Constant domain: Common across most |
| | | (CL) | IgG1 |
| | HC- Cys222- LC- | HC-LC-TGFβRII | Inter-chain peptide holding the heavy |
| | Cys214 | linkage | and light chain. Its presence confirms the |
| | | confirmation | integrity of HC and LC and TGFβRII |
| | | | fusion |
| TGFβRII -ECD | Cys258-Cys261 | Disulfide linkage | Confirms the structural integrity of the |
| | Cys268-Cys274 | confirmation | TGFβRII-ECD |
| | Cys278-Cys284 | | |
| | Cys291-Cys308 | | |
| | Cys328-Cys343 | | |
| | Cys345-Cys350 | | |

Out of the disulfide linkages in the TGFβRII ECD, two of the tryptic peptides showed the presence of multiple disulfide bonds (peaks 10 and 11 in Table 25). To identify the linkages, an additional characterization test was performed using multiple enzyme digest and MS 2 confirmation. MS 2 was also used to confirm the Cys258-Cys261 linkage.

### Table 25.

TABLE 25
Non-reduced di-sulfide linked tryptic digest of BCA101 DS GF19000040 and BCA101 DS BL.14.0901/R/17/021/F
DS with their expected and observed mass and corresponding retention time (RT)

| | | | Expected | Expected | BL.14.0901/R/17/021/F DS | | GF19000040 | |
|---|---|---|---|---|---|---|---|---|
| | Cysteine | Peak | Mass [Da] | Mass [Da] | RT | Mass | RT | Mass |
| Location | Linkages | Number | Mono | Ave[MH]+ | (min) | (Da) | (min) | (Da) |
| Heavy chain | Cys22-Cys95 | 1 | 5351.5 | 5355.9# | 115.0 | 7122.2 | 115.0 | 7123.0 |
| (Fab Domain) | Cys146-Cys202 | 2 | 7916.9 | 7922.8 | 142.6 | 7919.0 | 142.6 | 7919.0 |
| Heavy chain | H-Cys228- | 3 | 5454.8 | 5459.5 | 142.9 | 5456.6 | 142.9 | 5456.6 |
| (Hinge) | H-Cys228; | | | | | | | |
| | H-Cys231- | | | | | | | |
| | H-Cys231 | | | | | | | |
| Heavy chain | Cys263-Cys323 | 4 | 2328.1 | 2330.6 | 68.6 | 2329.4 | 68.6 | 2329.4 |
| (Fc Domain) | Cys369-Cys427 | 6 | 3844.8 | 3848.3 | 95.5 | 3846.4 | 95.5 | 3846.4 |
| Light chain | Cys23-Cys88 | 7 | 5203.3 | 5207.5 | 113.2 | 5205.0 | 113.2 | 5205.0 |
| (Fab Domain) | Cys134-Cys194 | 8 | 3555.7 | 3559.0 | 110.4 | 3557.2 | 110.5 | 3557.2 |
| | HC-Cys222- | 5 | 2603.1 | 2604.7 | 31.6 | 2605.4 | 31.5 | 2605.5 |
| | LC-Cys214 | | | | | | | |
| TGFβRII - | Cys258-Cys261 | 9 | 1370.6 | 1372.6 | 61.3 | 1372.8 | 61.3 | 1372.8 |
| ECD | Cys268-Cys274; | 10 | 5334.3# | 5320.9# | 140.7 | 7107.3 | 140.5 | 7107.8 |
| | Cys278-Cys284 | | | | | Glycosylated | | Glycosylated |
| | Cys291-Cys308 | | | | | (G2F:1769) | | (G2F:1769 |
| | Cys328-Cys343; | 11 | 4114.5 | 4115.5 | 93.7 | 4113.1 | 93.7 | 4113.1 |
| | Cys345-Cys350 | | | | | | | |

#contains Glycosylated peptide

Table 26 describes the expected mass of the peptide from multiple enzyme digestion along with their retention times (RT).

TABLE 26
Non-reduced di-sulfide linked multiple enzyme digest of BCA101 DS
GF19000040 batch and BCA101 BL.14.0901/R/17/021/F DS batch, their
expected and observed mass, and corresponding retention time

| | | | Expected | Expected | BL.14.0901/R/17/021/F DS | | GF19000040 | |
|---|---|---|---|---|---|---|---|---|
| | Cysteine | Enzyme | Mass [Da] | Mass [Da] | Mass | RT | Mass | RT |
| Location | Linkages | Used | Mono | Ave[MH]+ | (Da) | (min) | (Da) | (min) |
| TGFβRII- | Cys258-Cys261 | Asp-N | 1680.7 | 1681.9 | 1681.8 | 45.3 | 1681.8 | 45.2 |
| ECD | Cys268-Cys274 | Asp-N/PNGaseF | 1736.7 | 1737.9 | 1736.7 | 21.3 | 1736.8 | 21.4 |
| | Cys278-Cys284 | Confirmed by | 3775.8 | 3778.2 | Confirmed based on the only disulfide | | | |
| | | the process of | | | linkage possible as the other cysteines are | | | |
| | | elimination | | | shown to be linked by MS2 data from | | | |
| | | | | | AspN and Trp/GluC peptide digestion | | | |
| | Cys291-Cys308 | Trypsin/GluC | 3242.5 | 3244.7 | 3242.8 | 45.9 | 3242.8 | 45.9 |

| | | | | |
|---|---|---|---|---|
| Cys328-Cys343; | Trypsin/LysC/ | 4112.6 | 4115.4 | To be determined |
| Cys345-Cys350 | GluC/PNGaseF | | | (MS data confirms the peptide |
| | | | | mass with the disulfide bonds) |

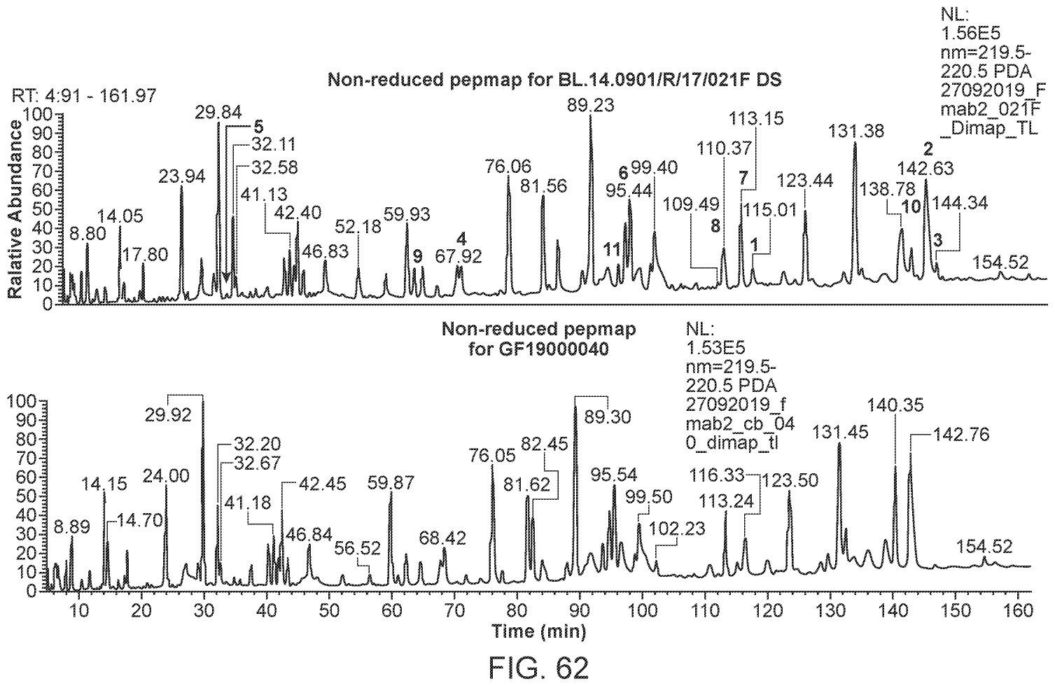

As observed in FIG. 62, the non-reduced PMF UV chromatogram of the BCA101 DS GF19000040 batch is comparable to the BCA101 BL.14.0901/R/17/021/F DS batch, and within specifications. The mass of disulfide linked peptides after proteolytic digestion by multiple enzymes for both the batches are tabulated in the Tables 25 and 26 and were found comparable across the batches and with the corresponding theoretical mass. Out of the six disulfide linkages in TGFβRII ECD, three were confirmed by MS 2 analysis—Cys258-Cys261 (AspN), Cys268-Cys274 (AspN/PNGaseF) and Cys291-Cys308 (Trp/GluC). Further, in one of the peptides generated by AspN digestion ( . . . NCSIT . . . TVCH), since Cys291-Cys308 is already confirmed from GluC digested peptide MS 2, the only possibility for the other disulfide bond is between Cys278-Cys284 which confirmed the fourth disulfide linkage. The mass of the corresponding peptide with disulfide bond has been confirmed and is comparable across the two batches. Therefore, the disulfide linked peptides in TGFβRII ECD were confirmed via multiple enzyme digestions and MS 2 confirmation.

### N-Glycan Analysis Using Normal Phase Liquid Chromatography (NP-HPLC) and ESI-MS

Heterogeneity of biotherapeutics expressed in mammalian cells is largely due to the post-translational modifications such as glycosylation in the conserved regions of proteins. N-linked glycosylation in monoclonal antibodies play an important role in ligand/antigen binding and its function such as antibody dependent cell cytotoxicity (ADCC), complement dependent cytotoxicity (CDC), and rate of clearance.

To determine the N-linked glycosylation of BCA101, the samples were denatured with SDS, deglycosylated using PNGaseF, the released glycans were labelled with 2-aminoanthranilic acid (2-AA), separated and quantified in FLD-NP-HPLC. The mass for each of the glycan species was determined using LC-ESI-MS.

Table 27 below provides the relative abundance of the observed glycan species. Mass identification of the glycan species acquired using LC-ESI-MS was confirmed by MS 2. The 2-AA labelled N-glycan NP-HPLC chromatogram shown in FIG. 63 and percent relative abundance (Table 27) of BCA101 DS GF19000040 batch and BCA101 DS BL.14.0901/R/17/021/F DS batch are comparable and within specifications.

TABLE 27
Relative abundance of the glycan species present in
two batches of BCA101 DS (BCA101 BL.14.0901/
R/17/021/F DS and BCA101 DS GF19000040)
% Relative Abundance

| Glycan species | BL.14.0901/R/17/021/F DS | GF19000040 |
|---|---|---|
| G0 − GN | 0.33 | 0.43 |
| G0f − GN | 1.73 | 2.09 |
| G0 | 1.24 | 1.49 |
| G0f | 20.05 | 18.95 |
| M5 | 1.90 | 2.85 |
| G1, G1f − GN | 0.45 | 0.77 |
| G0f + GN | 0.97 | 1.35 |
| G1f | 9.27 | 8.71 |
| G1fS1 − GN, M6 | 0.72 | 1.10 |
| G1fS1 | 0.17 | 0.31 |
| G1f + GN | 1.62 | 1.93 |
| G2f | 6.37 | 5.90 |
| G2fS1 | 13.35 | 12.69 |
| G2fS2 | 8.13 | 7.26 |
| Other species | 33.71 | 35.75 |

### Sialic Acid Content

Sialic acid exists in two forms: N-glycosylneuraminic acid (NGNA) and N-acetylneuraminic acid (NANA). Human glycosylated proteins contain the NANA form of sialic acid. Among the variety of monosaccharides present on Fc glycans, terminal sialic acids are particularly interesting, as they play different roles in monoclonal antibody function.

The half-life of a number of glycoproteins can be enhanced by sialylation, as sialic acid acts as a cap that hides the penultimate galactose residue recognized by the hepatic asialoglycoprotein receptor. Hence, from the perspective of obtaining a safe and reliable monoclonal antibody for various biotherapeutic applications, better understanding and monitoring of sialylated glycans is required.

In addition to influencing the biological and physiochemical properties of biopharmaceutical drugs, sialic acid moieties of the protein therapeutics play a major role in serum half-life because the galactose exposed as glycoproteins are endocytosed by hepatic asialo galactose receptors via receptor mediated endocytosis. The release of sialic acid is through acid hydrolysis of the monoclonal antibody, followed by clean up to remove the monoclonal antibody and fluorescent tag labelling of sialic acid species with OPD. The tagged samples are injected into the reversed-phase high-performance liquid chromatographic (RP-HPLC) column for analysis. Linear dynamic calibration range of the assay is determined by running sets of NANA standards at different concentrations.

The data overlay shown in FIG. 64 indicates that the peak corresponding to NGNA, based on the NGNA standard samples at 25 and 300 pmol, is absent in BCA101 DS, which shows only the peak corresponding to NANA (based on the peak seen in NANA standard sample). Therefore, the data shows the presence of NANA in BCA101 and that NGNA was not detected up to 20 pmol. As shown in Table 28, the average (n=4) sialic acid content in BCA101 DS (GF19000040) was estimated to be 8.8 moles/mole of protein and that of BCA101 DS (BL.14.0901/R/17/021/F DS) was determined to be 12.2 moles/mole of protein. This difference in average sialic acid content is within the method variability and has little influence on the biological function between the batches as shown in Functional assays as presented below.

TABLE 28
Sialic acid content measured in BCA101 DS batch
GF19000140 and batch BL.14.0901/R/17/021/F DS

| | Moles of NANA/Moles | Standard |
|---|---|---|
| Sample ID | of Protein (n = 4) | Deviation |
| GF19000040 | 8.8 | 2.3 |
| BL.14.0901/R/17/021/F DS | 12.2 | 2.5 |

### Product Related Substances—RP-HPLC

The reversed phase HPLC (RP-HPLC) method is employed for monitoring product related hydrophobic variants including post-translational modifications, oxidized protein, clipped variants or fragments, N-terminal cyclization, etc. Based on hydrophobicity proteins are separated in the column; with less hydrophobic proteins eluting earlier than highly hydrophobic variants.

The product related hydrophobic variants in BCA101 batches were categorized into Main peak, the peaks before main grouped together as total pre-peak, and the peaks post main peak were grouped together as total post-peaks. The RP profile and relative proportion of main peak, pre-peaks, and post-peaks were observed to be similar across the two BCA101 DS batches (GF19000140 and BL.14.0901/R/17/021/F DS) (Table 29). The total main peak content was observed to be 82.2% (GF19000140) and 81.3% (BL.14.0901/R/17/021/F DS), both of which are within the specification of not less than (NLT) 70%. The percent total pre-peaks was observed to be 4.9% (GF19000140) and 4.8% (BL.14.0901/R/17/021/F DS), both within the specification of not more than (NMT) 6.0%. Total post peaks content for was observed to be 12.9% (GF19000140) and 13.9% (BL.14.0901/R/17/021/F DS), both within the specification of not more than (NMT) 24.0%.

TABLE 29
RP-HPLC data of BCA101 DS batches GF19000040
and BL.14.0901/R/17/021/F DS

| | Pre- | Main | Post- |
|---|---|---|---|
| Sample | peaks % | Peak % | peaks % |
| GF19000040 | 4.9 | 82.2 | 12.9 |
| BL.14.0901/R/17/021/F DS | 4.8 | 81.3 | 13.9 |

### Inhibition of Proliferation (IOP)

BCA101 binds to EGFR on FaDu cancer cells. Varying the drug concentration enables a dose dependent inhibition of proliferation of cancer cells in this assay. The cell Titer-Glo® 2.0 Assay provides a homogeneous method to determine the number of viable cells in culture by quantitating the amount of ATP present, which indicates the presence of metabolically active cells. The read out is based on luminescence by mono-oxygenation of luciferin, which is catalyzed by luciferase in the presence of Mg2+ and ATP released by viable cells.

The GF19000040 batch was analyzed by three independent experiments with the BL.14.0901/R/17/021/F DS batch as a standard. The average relative potency from three experiments is described below in Table 30.

As shown in Table 30, the GF19000040 batch displayed an average relative potency of 0.99, which falls within the assay acceptance criteria of 0.8-1.25. The results indicate GF19000040 and BL.14.0901/R/17/021/F DS show similar potency for inhibition of FaDu cell proliferation.

TABLE 30
Average relative potency of BCA101
DS as measured by IOP assay

| Functional Assay | Batch | Average Relative Potency (N = 3) |
|---|---|---|
| IOP Assay | GF19000040 | 0.99 |

### Bi-Functional ELISA

As described above, a bi-functional ELISA assay was used to determine the binding efficacy of fusion moieties of BCA101 to its target protein, thus, to determine that both moieties in BCA101 are concurrently functional. FmAb2 has a TGFβRII ECD fused to an anti-EGFR monoclonal antibody at the C-terminus of the light chain. The TGFβRII ECD moiety binds to TGFβ 1 predominantly and the anti-EGFR binds to EGFR. Individual target binding ELISAs can only determine the biding affinity of each moiety independently but not the bi-functionality.

The GF19000040 batch was evaluated with BL.14.0901/R/17/021/F DS as a standard for their bi-functional activity. As shown in Table 31, GF19000040 the displayed relative potency value of 0.93 which is well within the acceptance criteria of 0.8-1.25. The result further indicates that GF19000040 displays similar potency for bi-functional activity compared to BL.14.0901/R/17/021/F DS.

TABLE 31
Average relative potency of BCA101 DS
as measured by bi-functional ELISA

| Functional Assay | Batch | Average Relative Potency (N = 3) |
|---|---|---|
| Bi-functional Assay | GF19000040 | 0.93 |

### Antibody-Dependent Cellular Cytotoxicity (ADCC):

ADCC assay evaluates the Fc functions of BCA101. The ADCC Reporter Bioassay is a bioluminescent reporter assay that uses an alternative readout at an earlier point in ADCC mechanism of action pathway by activating the gene transcription through the NFAT (nuclear factor of activated T-cells) pathway in the effector cell. The ADCC Reporter Bioassay is performed with the ADCC Bioassay Effector Cells Propagation Model (Promega, CAT #G7102) that allows cell banking and propagation of the cells. These cells are engineered Jurkat cells stably expressing the FcγRIIIa receptor, V158 (high affinity) variant. Biological activity in ADCC is quantified through the luciferase produced as a result of NFAT pathway activation. Luciferase activity in the effector cell is quantified with luminescence readout.

The GF19000040 batch was evaluated with FmAb2 IRS as standard for ADCC activity. As shown in Table 32, the GF19000040 batch displayed relative potency value of 1.23, which is within the acceptance criteria of 0.8-1.25 and also displayed the % CV of ≤20. The results indicated that GF19000040 batch displayed similar potency for ADCC activity compared to the BL.14.0901/R/17/021/F DS batch.

TABLE 32
Average relative potency of BCA101
DS as measured by ADCC assay

| | | Average Relative | |
|---|---|---|---|
| Functional Assay | Batch | Potency (N = 3) | % CV |
| ADCC | GF19000040 | 1.23 | 1.33 |

### TGFβ SMAD Assay

The TGFβ SMAD assay is a functional assay, which determines the functional activity of TGFβRII ECD arm of the fusion antibody. Briefly, the HEK293 cell line is engineered to determine the activity of TGFβ-SMAD signaling pathway. The cell line contains a firefly luciferase gene under the control of SMAD-responsive elements stably integrated into HEK293 cells. TGFβ proteins binds to receptors on the cell surface, initiating a signalling cascade that leads to phosphorylation and activation of SMAD2 and SMAD3, which then forms a complex with SMAD4. The SMAD complex then translocates to the nucleus and binds to the SMAD binding element (SBE) in the nucleus, leading to transcription and expression of TGFβ SMAD responsive genes. Stimulation with human TGFβ1 increases the luminescence signal which is neutralized in the presence of BCA101 in a dose dependent manner.

As shown in Table 33, the GF19000040 batch was evaluated with BL.14.0901/R/17/021/F DS as a standard. The GF19000040 batch displayed a relative potency value of 1.10, which is well within the accepted range of 0.80 to 1.25 and also displayed a % CV of ≤20. Further establishing that the GF19000040 batch is comparable with the BL.14.0901/R/17/021/F DS batch.

TABLE 33
Average relative potency of BCA101 DS as measured by TGFβ SMAD assay

| | | | | | | Average | | |
|---|---|---|---|---|---|---|---|---|
| | | Date of | Slope of | Relative | | Relative | | |
| Batch | Experiment No. | Readout | Standard | Potency | RCI % | Potency | SD | % CV |
| GF19000040 | DDL/BR.14.09015/19/014/18 | 16 Oct. 2019 | −0.6152 | 0.95924 | 23.01 | 1.10 | 0.14 | 13.05 |
| | DDL/BR.14.09015/19/014/18 | 6 Oct. 2019 | −0.64185 | 1.09536 | 21.34 | | | |
| | DDL/BR.14.09015/19/014/19 | 17 Oct. 2019 | −0.61357 | 1.24631 | 32.63 | | | |

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

What is claimed is:

1. A liquid pharmaceutical composition comprising:
a. a fusion protein that comprises a targeting moiety and an immunomodulatory moiety, wherein: i) said targeting moiety specifically binds human epidermal growth factor receptor (hEGFR); and (ii) said immunomodulatory moiety comprises an amino acid sequence of the extracellular domain of human transforming growth factor-beta receptor II (hTGFβRII);
b. a buffer present at a concentration from 5 mM to 30 mM; and
c. a tonifying agent present at a concentration from 4% w/v to 10% w/v;
wherein, said liquid pharmaceutical composition has a pH from 5.5 to 7.0.

2. The liquid pharmaceutical composition of claim 1, wherein said buffer is a citrate phosphate buffer, citrate buffer, succinate buffer, or histidine buffer.

3. The liquid pharmaceutical composition of any one of the preceding claims, wherein said buffer is a citrate phosphate buffer.

4. The liquid pharmaceutical composition of any one of the preceding claims, wherein said buffer is present at a concentration from 5 mM to 25 mM, 5 mM to 20 mM, 5 mM to 15 mM, 5 mM to 10 mM, or 10 mM to 30 mM.

5. The liquid pharmaceutical composition of any one of the preceding claims, wherein said buffer is present at a concentration from 5 mM to 15 mM.

6. The liquid pharmaceutical composition of any one of the preceding claims, wherein said buffer is present at a concentration of 5 mM, 10 mM, 15 mM, 20 mM, 25 mM, or 30 mM.

7. The liquid pharmaceutical composition of any one of the preceding claims, wherein said buffer is present at a concentration of 10 mM.

8. The liquid pharmaceutical composition of any one of the preceding claims, wherein said buffer comprises 10 mM phosphate citrate.

9. The liquid pharmaceutical composition of any one of the preceding claims, wherein said tonifying agent is sucrose or trehalose.

10. The liquid pharmaceutical composition of any one of the preceding claims, wherein said tonifying agent is a saccharide.

11. The liquid pharmaceutical composition of any one of the preceding claims, wherein said tonifying agent is a disaccharide.

12. The liquid pharmaceutical composition of any one of the preceding claims, wherein said tonifying agent is sucrose.

13. The liquid pharmaceutical composition of any one of the preceding claims, wherein said tonifying agent is present at a concentration from 5% w/v to 10% w/v, 6% w/v to 10% w/v, 7% w/v to 10% w/v, 8% w/v to 10% w/v, 5% w/v to 9% w/v, 5% w/v to 8% w/v, 6% w/v to 9% w/v, 6% w/v to 8% w/v, 7% w/v to 9% w/v, or 7% w/v to 8% w/v.

14. The liquid pharmaceutical composition of any one of the preceding claims, wherein said tonifying agent is present at a concentration from 5% w/v to 8% w/v.

15. The liquid pharmaceutical composition of any one of the preceding claims, wherein said tonifying agent is present at a concentration of 5% w/v, 6% w/v, 7% w/v, 8% w/v, 9% w/v, or 10% w/v.

16. The liquid pharmaceutical composition of any one of the preceding claims, wherein said tonifying agent is present at a concentration of 8% w/v.

17. The liquid pharmaceutical composition of any one of the preceding claims, wherein said tonifying agent is sucrose and is present at a concentration of 8% w/v.

18. The liquid pharmaceutical composition of any one of the preceding claims, further comprising a surfactant.

19. The liquid pharmaceutical composition of claim 18, wherein said surfactant comprises polysorbate 20, polysorbate 40, polysorbate 60, or polysorbate 80.

20. The liquid pharmaceutical composition of claim 19, wherein said surfactant comprises polysorbate 20.

21. The liquid pharmaceutical composition of any one of claims 18-20, wherein said surfactant is present at a concentration from 0.005-0.1% w/v.

22. The liquid pharmaceutical composition of any one of claims 18-21, wherein said surfactant is present at a concentration from 0.01-0.1% w/v, 0.02-0.1% w/v, 0.01-0.9% w/v, 0.01-0.8% w/v, 0.01-0.7% w/v, 0.01-0.6% w/v, 0.01-0.5% w/v, 0.01-0.4% w/v, 0.01-0.3% w/v, 0.01-0.2% w/v, 0.01-0.1% w/v, 0.02-0.9% w/v, 0.02-0.8% w/v, 0.02-0.7% w/v, 0.02-0.6% w/v, 0.02-0.5% w/v, 0.02-0.4% w/v, 0.02-0.3% w/v, 0.02-0.2% w/v, 0.02-0.1% w/v, 0.005-0.9% w/v, 0.005-0.8% w/v, 0.005-0.7% w/v, 0.005-0.6% w/v, 0.005-0.5% w/v, 0.005-0.4% w/v, 0.005-0.3% w/v, 0.005-0.2% w/v, or 0.005-0.1% w/v.

23. The liquid pharmaceutical composition of any one of claims 18-22, wherein said surfactant is present at a concentration of 0.01% w/v, 0.02% w/v, 0.03% w/v, 0.04% w/v, 0.05% w/v, 0.06% w/v, 0.07% w/v, 0.08% w/v, 0.09% w/v, or 0.1% w/v.

24. The liquid pharmaceutical composition of any one of claims 18-23, wherein said surfactant is present at a concentration of 0.02% w/v.

25. The liquid pharmaceutical composition of any one of claims 18-24, wherein said surfactant is polysorbate 20 and is present at a concentration of 0.02% w/v.

26. The liquid pharmaceutical composition of any one of the preceding claims, wherein said liquid pharmaceutical composition has a pH from 5.5 to 7.0, 6.0 to 7.0, 5.5 to 6.5, 5.5 to 6.0, or 6.0 to 6.5.

27. The liquid pharmaceutical composition of any one of the preceding claims, wherein said liquid pharmaceutical composition has a pH from 6.0 to 6.5.

28. The liquid pharmaceutical composition of any one of the preceding claims, wherein said liquid pharmaceutical composition has a pH of 5.5, 6.0, 6.5, or 7.0.

29. The liquid pharmaceutical composition of any one of the preceding claims, wherein said liquid pharmaceutical composition has a pH of 6.0.

30. The liquid pharmaceutical composition of any one of the preceding claims, wherein said liquid pharmaceutical composition has an osmolality from 150 mOsmol/kg to 400 mOsmol/kg.

31. The liquid pharmaceutical composition of any one of the preceding claims, wherein said liquid pharmaceutical composition has an osmolality from 150 mOsmol/kg to 350 mOsmol/kg, 150 mOsmol/kg to 300 mOsmol/kg, 200 mOsmol/kg to 400 mOsmol/kg, 250 mOsmol/kg to 400 mOsmol/kg, 300 mOsmol/kg to 400 mOsmol/kg, 300 mOsmol/kg to 350 mOsmol/kg, 250 mOsmol/kg to 350 mOsmol/kg, or 250 mOsmol/kg to 300 mOsmol/kg.

32. The liquid pharmaceutical composition of any one of the preceding claims, wherein said liquid pharmaceutical composition has an osmolality from 250 mOsmol/kg to 350 mOsmol/kg.

33. The liquid pharmaceutical composition of any one of the preceding claims, wherein said liquid pharmaceutical composition has an osmolality of 250 mOsmol/kg, 300 mOsmol/kg, or 300 mOsmol/kg.

34. The liquid pharmaceutical composition of any one of the preceding claims, wherein said liquid pharmaceutical composition has an osmolality of 300 mOsmol/kg.

35. The liquid pharmaceutical composition of any one of the preceding claims, wherein said liquid pharmaceutical composition is stable for at least 12, 18, or 24 months when stored at −20° C.

36. The liquid pharmaceutical composition of any one of the preceding claims, wherein said liquid pharmaceutical composition is stable for at least 12, 18, or 24 months when stored at 2-8° C.

37. The liquid pharmaceutical composition of any one of the preceding claims, wherein the concentration of said fusion protein in said liquid pharmaceutical composition is substantially the same for at least 12, 18, or 24 months when stored at −20° C.

38. The liquid pharmaceutical composition of any one of the preceding claims, wherein the concentration of said fusion protein in said liquid pharmaceutical composition is substantially the same for at least 12, 18, or 24 months when stored at 2-8° C.

39. The liquid pharmaceutical composition of any one of the preceding claims, wherein the concentration of said fusion protein in said liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 12, 18, or 24 months at −20° C.

40. The liquid pharmaceutical composition of any one of the preceding claims, wherein the concentration of said fusion protein in said liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 12, 18, or 24 months at 2-8° C.

41. The liquid pharmaceutical composition of any one of the preceding claims, wherein said liquid pharmaceutical composition is stable upon 1, 2, 3, 4, or 5 cycles of freezing and thawing.

42. The liquid pharmaceutical composition of any one of the preceding claims, wherein said fusion protein retains bifunctional activity as measured by bifunctional enzyme-linked immunosorbent assay (ELISA) for at least 12, 18, or 24 months when stored at −20° C.

43. The liquid pharmaceutical composition of any one of the preceding claims, wherein said fusion protein retains bifunctional activity as measured by bifunctional ELISA for at least 12, 18, or 24 months when stored at 2-8° C.

44. The liquid pharmaceutical composition of any one of the preceding claims, wherein said liquid pharmaceutical composition comprises less than 10%, 9%, 8%, 7%, 6%, 5%, 4%, 3%, 2%, or 1% of said fusion protein in aggregate form.

45. The liquid pharmaceutical composition of any one of the preceding claims, wherein said liquid pharmaceutical composition has at least one feature selected from the group consisting of
a. increased shelf life
b. increased temperature stability,
c. decreased formation of aggregates,
d. increased chemical stability, and/or
e. decreased fragmentation
f. decreased viscosity,
after 12, 18, or 24 months of storage at −20° C. or 2-8° C., as compared to a control formulation.

46. The liquid pharmaceutical composition of any one of the preceding claims, wherein said liquid pharmaceutical composition has at least one feature selected from the group consisting of:
a. decreased percentage of aggregates as measured by size exclusion chromatography (SEC),
b. higher percentage of monomers as measured by SEC, and/or
c. lower turbidity value in nephelometry units (NTU),
after 12, 18, or 24 months of storage at −20° C. or 2-8° C., as compared to the reference formulation.

47. The liquid pharmaceutical composition of any one of the preceding claims, wherein said fusion protein is present at a concentration from 5-50 mg/ml, 5-40 mg/ml, 5-30 mg/ml, 5-25 mg/ml, 10-50 mg/ml, 20-50 mg/ml, 25-50 mg/ml, 20-50 mg/ml, 20-40 mg/ml, 20-30 mg/ml, 25-50 mg/ml, 25-40 mg/ml, or 25-30 mg/ml.

48. The liquid pharmaceutical composition of any one of the preceding claims, wherein said fusion protein is present at a concentration from 20-30 mg/ml.

49. The liquid pharmaceutical composition of any one of the preceding claims, wherein said fusion protein is present at a concentration of 5 mg/ml, 10 mg/ml, 15 mg/ml, 20 mg/ml, 25 mg/ml, 30 mg/ml, 35 mg/ml, 40 mg/ml, 45 mg/ml, or 50 mg/ml.

50. The liquid pharmaceutical composition of any one of the preceding claims, wherein said fusion protein is present at a concentration of 25 mg/ml.

51. The liquid pharmaceutical composition of any one of the preceding claims, wherein said targeting moiety that specifically binds hEGFR comprises an antibody or functional fragment or functional variant thereof.

52. The liquid pharmaceutical composition of claim 51, wherein said antibody, or functional fragment or functional variant thereof, that specifically binds hEGFR is a full-length antibody, a single chain variable fragment (scFv), a scFv2, a scFv-Fc, a Fab, a Fab′, a F(ab′)2, or a F(v).

53. The liquid pharmaceutical composition of claim 51 or 52, wherein said antibody, or functional fragment or functional variant thereof, that specifically binds hEGFR comprises a VH that comprises VH CDR1, VH CDR2, and VH CDR3, wherein
a. VH CDR1 comprises an amino acid sequence at least 95%, 96%, 97%, 98%, 99%, or 100% identical to the amino acid sequence of SEQ ID NO: 1;
b. VH CDR2 comprises an amino acid sequence at least 95%, 96%, 97%, 98%, 99%, or 100% identical to the amino acid sequence of SEQ ID NO: 2; and
c. VH CDR3 comprises an amino acid sequence at least 95%, 96%, 97%, 98%, 99%, or 100% identical to the amino acid sequence of SEQ ID NO: 3.

54. The liquid pharmaceutical composition of any one of claims 51-53, wherein said antibody, or functional fragment or functional variant thereof, that specifically binds hEGFR comprises a VL that comprises a VL CDR1, a VL CDR2, and a VL CDR3, wherein
a. VL CDR1 comprises an amino acid sequence at least 95%, 96%, 97%, 98%, 99%, or 100% identical to the amino acid sequence of SEQ ID NO: 4;
b. VL CDR2 comprises an amino acid sequence at least 95%, 96%, 97%, 98%, 99%, or 100% identical to the amino acid sequence of SEQ ID NO: 5; and
c. VL CDR3 comprises an amino acid sequence at least 95%, 96%, 97%, 98%, 99%, or 100% identical to the amino acid sequence of SEQ ID NO: 6.

55. The liquid pharmaceutical composition of any one of claims 51-54, wherein said antibody, or functional fragment or functional variant thereof, that specifically binds hEGFR comprises a VH that comprises an amino acid sequence at least 95%, 96%, 97%, 98%, 99%, or 100% identical to the amino acid sequence of SEQ ID NO: 7.

56. The liquid pharmaceutical composition of any one of claims 51-55, wherein said antibody, or functional fragment or functional variant thereof, that specifically binds hEGFR comprises a VL that comprises an amino acid sequence at least 95%, 96%, 97%, 98%, 99%, or 100% identical to the amino acid sequence of SEQ ID NO: 8.

57. The liquid pharmaceutical composition of any one of claims 51-56, wherein said antibody, or functional fragment or functional variant thereof, that specifically binds hEGFR comprises a heavy chain that comprises an amino acid sequence at least 95%, 96%, 97%, 98%, 99%, or 100% identical to the amino acid sequence of SEQ ID NO: 9.

58. The liquid pharmaceutical composition of any one of claims 51-57, wherein said antibody, or functional fragment or functional variant thereof, that specifically binds hEGFR consists of a heavy chain that comprises an amino acid sequence at least 95%, 96%, 97%, 98%, 99%, or 100% identical to the amino acid sequence of SEQ ID NO: 10.

59. The liquid pharmaceutical composition of any one of claims 51-57, wherein said antibody, or functional fragment or functional variant thereof, that specifically binds hEGFR comprises a heavy chain that consists of an amino acid sequence at least 95%, 96%, 97%, 98%, 99%, or 100% identical to the amino acid sequence of SEQ ID NO: 9.

60. The liquid pharmaceutical composition of any one of claims 51-57, wherein said antibody, or functional fragment or functional variant thereof, that specifically binds hEGFR consists of a heavy chain that consists of an amino acid sequence at least 95%, 96%, 97%, 98%, 99%, or 100% identical to the amino acid sequence of SEQ ID NO: 10.

61. The liquid pharmaceutical composition of any one of claims 51-60, wherein said antibody, or functional fragment or functional variant thereof, that specifically binds hEGFR comprises a light chain that comprises an amino acid sequence at least 95%, 96%, 97%, 98%, 99%, or 100% identical to the amino acid sequence of SEQ ID NO: 11.

62. The liquid pharmaceutical composition of any one of claims 51-61, wherein said antibody, or functional fragment or functional variant thereof, that specifically binds hEGFR consists of a light chain that consists of an amino acid sequence at least 95%, 96%, 97%, 98%, 99%, or 100% identical to the amino acid sequence of SEQ ID NO: 11.

63. The liquid pharmaceutical composition of claim 51, wherein said antibody, or functional fragment or functional variant thereof, that specifically binds hEGFR comprises cetuximab or panitumumab, or a functional fragment or functional variant of any of the foregoing.

64. The liquid pharmaceutical composition of any one of the preceding claims, wherein said immunomodulatory moiety comprises an amino acid sequence at least 95%, 96%, 97%, 98%, 99%, or 100% identical to the amino acid sequence of SEQ ID NO: 23.

65. The liquid pharmaceutical composition of any one of the preceding claims, wherein said immunomodulatory moiety consists of an amino acid sequence at least 95%, 96%, 97%, 98%, 99%, or 100% identical to the amino acid sequence of SEQ ID NO: 23.

66. The liquid pharmaceutical composition of any one of the preceding claims, wherein said immunomodulatory moiety is indirectly fused to said targeting moiety.

67. The liquid pharmaceutical composition of claim 66, wherein said immunomodulatory moiety is indirectly fused to said targeting moiety via a peptide linker.

68. The liquid pharmaceutical composition of claim 67, wherein said immunomodulatory moiety is indirectly fused to said targeting moiety via a peptide linker of sufficient length such that said immunomodulatory moiety and said targeting moiety can simultaneously bind the respective targets.

69. The liquid pharmaceutical composition of claim 66 or 67, wherein said linker comprises the amino acid sequence of SEQ ID NO: 24, 25, 26, 27, or 28.

70. The liquid pharmaceutical composition of any one of claims 67-69, wherein said linker comprises the amino acid sequence of SEQ ID NO: 24.

71. The liquid pharmaceutical composition of any one of claims 67-69, wherein said linker consists of the amino acid sequence of SEQ ID NO: 24.

72. The liquid pharmaceutical composition of any one of claims 1-71, wherein said immunomodulatory moiety is fused to the C terminus of said targeting moiety.

73. The liquid pharmaceutical composition of any one of claims 1-71, wherein said immunomodulatory moiety is fused to the N terminus of said targeting moiety.

74. The liquid pharmaceutical composition of any one of the preceding claims, wherein said targeting moiety is an antibody that comprises a light chain and a heavy chain, and wherein said immunomodulatory moiety is fused to the C terminus of said heavy chain of said targeting moiety.

75. The liquid pharmaceutical composition of any one of claims 1-74, wherein said targeting moiety is an antibody that comprises a light chain and a heavy chain, and wherein said immunomodulatory moiety is fused to the C terminus of said light chain of said targeting moiety.

76. The liquid pharmaceutical composition of any one of the preceding claims, wherein said targeting moiety is an antibody specifically binds hEGFR that comprises a heavy chain that comprises an amino acid sequence at least 95%, 96%, 97%, 98%, 99%, or 100% identical to the amino acid sequence of SEQ ID NO: 10, and a light chain that comprises an amino acid sequence at least 95%, 96%, 97%, 98%, 99%, or 100% identical to the amino acid sequence of SEQ ID NO: 11, and wherein said immunomodulatory moiety comprises an amino acid sequence at least 95%, 96%, 97%, 98%, 99%, or 100% identical to the amino acid sequence of SEQ ID NO: 23, and wherein the N terminus of said immunomodulatory moiety is fused indirectly through a linker to the C terminus of said heavy chain or said light chain, and wherein said linker comprises an amino acid sequence at least 95%, 96%, 97%, 98%, 99%, or 100% identical to the amino acid sequence of SEQ ID NO: 24.

77. The liquid pharmaceutical composition of any one of the preceding claims, wherein said targeting moiety is an antibody specifically binds hEGFR that comprises a heavy chain that comprises an amino acid sequence at least 95%, 96%, 97%, 98%, 99%, or 100% identical to the amino acid sequence of SEQ ID NO: 10, and a light chain that comprises an amino acid sequence at least 95%, 96%, 97%, 98%, 99%, or 100% identical to the amino acid sequence of SEQ ID NO: 11, and wherein said immunomodulatory moiety comprises an amino acid sequence at least 95%, 96%, 97%, 98%, 99%, or 100% identical to the amino acid sequence of SEQ ID NO: 23, and wherein the N terminus of said immunomodulatory moiety is fused indirectly through a linker to the C terminus of said light chain, and wherein said linker comprises an amino acid sequence at least 95%, 96%, 97%, 98%, 99%, or 100% identical to the amino acid sequence of SEQ ID NO: 24.

78. The liquid pharmaceutical composition of any one of the preceding claims, wherein said targeting moiety comprises an antibody that comprises a heavy chain comprising an amino acid sequence at least 95%, 96%, 97%, 98%, 99%, or 100% identical to the amino acid sequence of SEQ ID NO: 10; and a light chain comprising an amino acid sequence at least 95%, 96%, 97%, 98%, 99%, or 100% identical to the amino acid sequence of SEQ ID NO: 29.

79. The liquid pharmaceutical composition of any one of the preceding claims, wherein said liquid pharmaceutical composition is sterile.

80. A liquid pharmaceutical composition comprising:
a. a fusion protein that comprises a targeting moiety and an immunomodulatory moiety, wherein: i) said targeting moiety specifically binds hEGFR; and (ii) said immunomodulatory moiety comprises an amino acid sequence of the hTGFβRII;
b. from 5 mM to 20 mM citrate phosphate buffer; and
c. from 6% w/v to 10% w/v sucrose;
wherein said liquid pharmaceutical composition has a pH of from 5.5 to 6.5.

81. The liquid pharmaceutical composition of claim 80, wherein said targeting moiety comprises an antibody that comprises a heavy chain comprising an amino acid sequence at least 95%, 96%, 97%, 98%, 99%, or 100% identical to the amino acid sequence of SEQ ID NO: 10; and a light chain comprising an amino acid sequence at least 95%, 96%, 97%, 98%, 99%, or 100% identical to the amino acid sequence of SEQ ID NO: 29.

82. The liquid pharmaceutical composition of claim 80 or 81, wherein said fusion protein is present at a concentration of 25 mg/ml.

83. The liquid pharmaceutical composition of any one of claims 80-82, further comprising from 0.01-0.05% w/v polysorbate 20.

84. A liquid pharmaceutical composition comprising:
a. a fusion protein that comprises a targeting moiety and an immunomodulatory moiety, wherein: i) said targeting moiety specifically binds hEGFR; and (ii) said immunomodulatory moiety comprises an amino acid sequence of the extracellular domain of hTGFβRII;
b. 10 mM citrate phosphate buffer; and
c. 8% w/v sucrose;
wherein said liquid pharmaceutical composition has a pH of 6.0±0.3.

85. The liquid pharmaceutical composition of claim 84, further comprising from 0.02% w/v polysorbate 20.

86. The liquid pharmaceutical composition of claim 84 or 85, wherein said targeting moiety comprises an antibody that comprises a heavy chain comprising an amino acid sequence at least 95%, 96%, 97%, 98%, 99%, or 100% identical to the amino acid sequence of SEQ ID NO: 10; and a light chain comprising an amino acid sequence at least 95%, 96%, 97%, 98%, 99%, or 100% identical to the amino acid sequence of SEQ ID NO: 29.

87. The liquid pharmaceutical composition of any one of claims 84-86, wherein said fusion protein is present at a concentration of 25 mg/ml.

88. A liquid pharmaceutical composition comprising:
a. 25 mg/mL of a fusion protein that comprises a targeting moiety and an immunomodulatory moiety, wherein said targeting moiety comprises an antibody that comprises a heavy chain comprising an amino acid sequence at least 95%, 96%, 97%, 98%, 99%, or 100% identical to the amino acid sequence of SEQ ID NO: 10; and a light chain comprising an amino acid sequence at least 95%, 96%, 97%, 98%, 99%, or 100% identical to the amino acid sequence of SEQ ID NO: 29;
b. 10 mM citrate phosphate buffer;
c. 8% w/v sucrose; and
d. 0.02% w/v polysorbate 20;
wherein said liquid pharmaceutical composition has a pH of 6.0±0.3.

89. A method of treating human cancer in a subject having cancer, said method comprising administering to said subject the liquid pharmaceutical composition of any one of claims 1-86.

90. The method of claim 89, wherein said liquid pharmaceutical composition is administered in an amount effective to treat said cancer.

91. The method of claim 89 or 90, wherein said fusion protein is administered to said human subject at a dose from 50 mg to 2000 mg.

92. The method of any one of claims 89-91, wherein said fusion protein is administered to said human subject at a dose of 50 mg, 60 mg, 64 mg, 100 mg, 150 mg, 200 mg, 240 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900, or 2000 mg.

93. The method of any one of claims 89-92, wherein said fusion protein is administered to said human subject at a dose of 64 mg, 240 mg, 800 mg, or 1600 mg.

94. The method of any one of claims 89-93, wherein said fusion protein is administered to said human subject every 1, 2, 3, or 4 weeks.

95. The method of claim 94, wherein said fusion protein is administered to said human subject every week.

96. The method of claim 94, wherein said fusion protein is administered to said human subject every 3 weeks.

97. The method of any one of claims 89-96, wherein the administering step comprises intravenously injecting the liquid pharmaceutical composition.

98. The method of any one of claims 89-97, wherein said cancer is a solid tumor.

99. The method of any one of claims 89-98, wherein said cancer is metastatic, recurrent, refractory, or any combination thereof.

100. The method of any one of claims 89-99, wherein said cancer comprises cancer cells that contain a genomic amplification of the EGFR gene, e.g., as detected by biopsy and fluorescence in situ hybridization.

101. The method of any one of claims 89-100, wherein said cancer comprises cancer cells that contain a genomic modification in the KRAS gene.

102. The method of claim 101, wherein said modification in the KRAS gene is a G12D substitution.

103. The method of claim 101, wherein said modification in the KRAS gene is a G13D modification.

104. The method of any one of claims 89-103, wherein said cancer is selected from the group consisting of eye, stomach, colon, rectum, colorectal, breast cancer, anal cancer, pancreatic cancer, thyroid cancer, liver cancer, ovarian cancer, lung cancer, skin cancer, brain cancer, spinal cord cancer, head cancer, and neck cancer.

105. The method of any one of claims 89-104, wherein said cancer is lung cancer.

106. The method of claim 105, wherein said cancer is squamous cell lung cancer (SqCLC).

107. The method of claim 106, wherein said SqCLC comprises cancer cells that does not express detectable levels of programmed death-ligand 1, as measured by a biopsy.

108. The method of claim 106 or 107, wherein said SqCLC comprises cancer cells that contain a genomic amplification of the EGFR gene, e.g., as detected by biopsy and fluorescence in situ hybridization.

109. The method of any one of claims 89-104, wherein said cancer is colorectal cancer.

110. The method of claim 109, wherein said colorectal cancer is RAS wild-type microsatellite stable Colorectal Carcinoma (RAS WT MSS CRC).

111. The method of any one of claims 89-104, wherein said cancer is breast cancer.

112. The method of claim 111, wherein said cancer is triple negative breast cancer (TNBC).

113. The method of any one of claims 89-104, wherein said cancer is a spinal cord cancer.

114. The method of claim 113, wherein said cancer of the spinal cord is a chordoma.

115. The method of any one of claims 89-104, wherein said cancer is a cancer of the eye.

116. The method of claim 115, wherein said cancer of the eye is a melanoma of the eye.

117. The method of any one of claims 89-104, wherein said cancer is a brain cancer.

118. The method of claim 117, wherein said brain cancer is a glioblastoma.

119. The method of any one of claims 89-104, wherein said cancer is ovarian cancer.

120. The method of claim 119, wherein said ovarian cancer is epithelial ovarian cancer.

121. The method of any one of claims 89-104, wherein said cancer is liver cancer.

122. The method of claim 121, wherein said liver cancer is hepatocellular carcinoma (HCC).

123. The method of any one of claims 89-104, wherein said cancer is thyroid cancer.

124. The method of claim 123, wherein said thyroid cancer is anaplastic thyroid cancer (ATC).

125. The method of any one of claims 89-104, wherein said cancer is pancreatic cancer.

126. The method of any one of claims 89-104, wherein said cancer is stomach cancer.

127. The method of any one of claims 89-104, wherein said cancer is head and neck cancer.

128. The method of claim 127, wherein said cancer is head and neck squamous cell carcinoma (HNSCC).

129. The method of any one of claims 89-104, wherein said cancer is anal cancer.

130. The method of claim 129, wherein said anal cancer is squamous cell carcinoma of anal canal (SCCAC).

131. A method of manufacturing a liquid pharmaceutical composition comprising:
a. culturing mammalian cells having stably incorporated into their genome one or more nucleic acids encoding a fusion protein that comprises a targeting moiety and an immunomodulatory moiety, wherein: i) said targeting moiety specifically binds hEGFR; and (ii) said immunomodulatory moiety comprises an amino acid sequence of the extracellular domain of hTGFβRII in a cell culture medium such that the cells secrete said fusion protein into the cell culture medium;
b. purifying the fusion protein from the cell culture media; and
c. preparing the pharmaceutical composition according to any one of claims 1-88.


SEQUENCE LISTING (34 sequences)

SEQ ID NO: 1 - protein, 5 aa, Artificial Sequence
  Synthetic Construct
NYGVH

SEQ ID NO: 2 - protein, 16 aa, Artificial Sequence
  Synthetic Construct
VIWSGGNTDYNTPFTS

SEQ ID NO: 3 - protein, 11 aa, Artificial Sequence
  Synthetic Construct
ALTYYDYEFAY

SEQ ID NO: 4 - protein, 11 aa, Artificial Sequence
  Synthetic Construct
RASQSIGTNIH

SEQ ID NO: 5 - protein, 7 aa, Artificial Sequence
  Synthetic Construct
YASESIS

SEQ ID NO: 6 - protein, 9 aa, Artificial Sequence
  Synthetic Construct
QQNNNWPTT

SEQ ID NO: 7 - protein, 119 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 8 - protein, 107 aa, Artificial Sequence
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 9 - protein, 449 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 448 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Artificial Sequence
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 7 aa, Artificial Sequence
  Synthetic Construct
SGDYYWT

SEQ ID NO: 13 - protein, 16 aa, Artificial Sequence
  Synthetic Construct
HIYYSGNTNYNPSLKS

SEQ ID NO: 14 - protein, 9 aa, Artificial Sequence
  Synthetic Construct
DRVTGAFDI

SEQ ID NO: 15 - protein, 11 aa, Artificial Sequence
  Synthetic Construct
QASQDISNYLN

SEQ ID NO: 16 - protein, 7 aa, Artificial Sequence
  Synthetic Construct
DASNLET

SEQ ID NO: 17 - protein, 9 aa, Artificial Sequence
  Synthetic Construct
QHFDHLPLA

SEQ ID NO: 18 - protein, 119 aa, Artificial Sequence [sequence omitted]

SEQ ID NO: 19 - protein, 107 aa, Artificial Sequence [sequence omitted]

SEQ ID NO: 20 - protein, 445 aa, Artificial Sequence
  Synthetic Construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 444 aa, Artificial Sequence
  Synthetic Construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 22 - protein, 214 aa, Artificial Sequence
  Synthetic Construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 137 aa, Artificial Sequence
  Synthetic Construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 24 - protein, 15 aa, Artificial Sequence
  Synthetic Construct
GGGGSGGGGSGGGGS

SEQ ID NO: 25 - protein, 19 aa, Artificial Sequence
  Synthetic Construct
GGGGSGGGGSGGGGSGGGS

SEQ ID NO: 26 - protein, 23 aa, Artificial Sequence
  Synthetic Construct
GGGGSGGGGSGGGGSGGGSGGGS

SEQ ID NO: 27 - protein, 10 aa, Artificial Sequence
  Synthetic Construct
GGGGSGGGGS

SEQ ID NO: 28 - protein, 5 aa, Artificial Sequence
  Synthetic Construct
GGGGS

SEQ ID NO: 29 - protein, 366 aa, Artificial Sequence
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 30 - protein, 600 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 31 - protein, 601 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 32 - protein, 366 aa, Artificial Sequence
  Synthetic Construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 33 - protein, 597 aa, Artificial Sequence
  Synthetic Construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 34 - protein, 596 aa, Artificial Sequence
  Synthetic Construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD